(19) United States
(12) Patent Application Publication
(10) Pub. No.: US 2011/0059948 A1
(43) Pub. Date: Mar. 10, 2011

(54) QUINOLINE, NAPHTHALENE AND CONFORMATIONALLY CONSTRAINED QUINOLINE OR NAPHTHALENE DERIVATIVES AS ANTI-MYCOBACTERIAL AGENTS

(76) Inventors: Jyoti Chattopadhyaya, Uppsal (SE); Ram Shankar Upadhayaya, Pune (IN)

(21) Appl. No.: 12/812,809

(22) PCT Filed: Jan. 9, 2009
(86) PCT No.: PCT/SE2009/050008

§ 371 Date: Jul. 14, 2010

Related U.S. Application Data
(62) Division of application No. 61/033,055, filed on Mar. 3, 2008.

(30) Foreign Application Priority Data
Jan. 14, 2008 (IN) 117/CHE/208

(51) Int. Cl. A61K 31/55; C07D 471/04; A61K 31/4375; C07D 221/18; A61K 31/473; C07D 401/12; C07D 401/14; A61K 31/496; C07D 215/227; A61K 31/47; C07D 413/10; A61K 31/5377; A61P 31/04

(52) U.S. Cl. 514/215; 540/576; 546/70; 514/285; 546/77; 514/284; 544/361; 514/253.02; 546/157; 514/312; 544/128; 514/235.2

(57) ABSTRACT

The invention relates to a compound of general formula I, II, III, IV, V, VI, VII, VIII, IX, X or a tautomer and the stereochemically isomeric forms thereof or pharmaceutically acceptable salts thereof, a N-oxide form thereof or a pro-drug thereof. The compound is usable as a medicament for the treatment of mycobacterial disease.

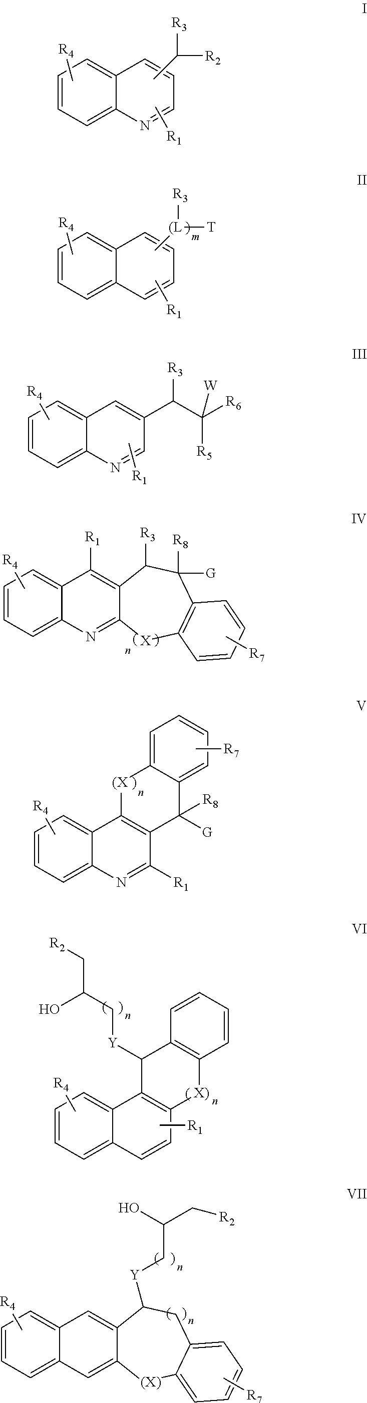

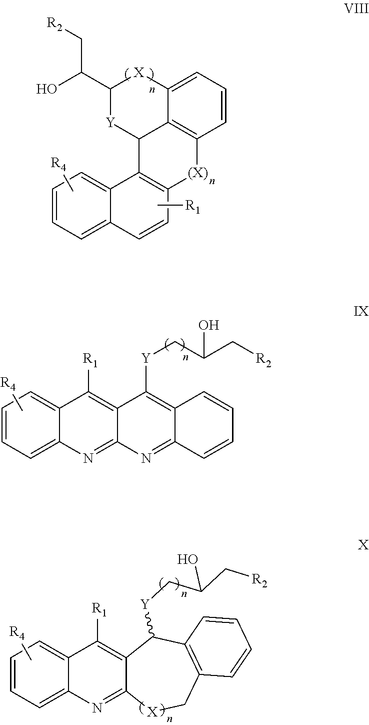

### FIELD OF THE INVENTION

The present invention relates to novel Quinoline, Non-quinoline (naphthalene) and their Conformationally-constrained derivatives, designated by general formula I, II, III, IV, V, VI, VII, VIII, IX, X and their pharmaceutically acceptable salts, possessing excellent

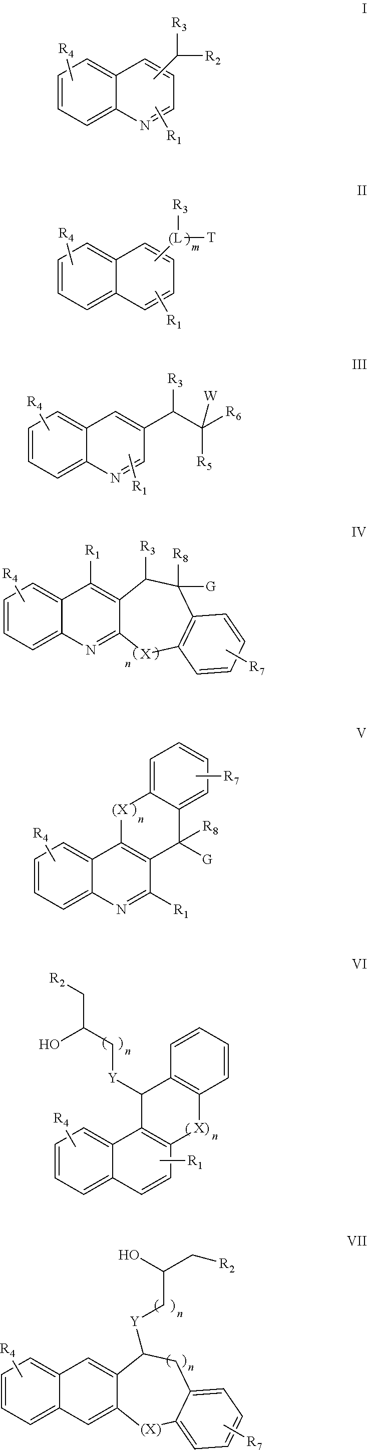

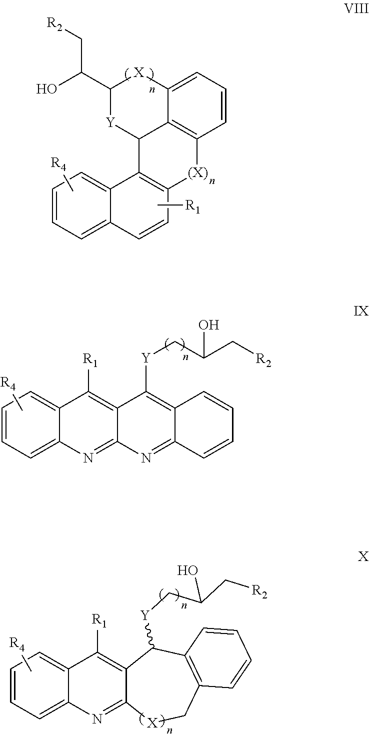

anti-mycobacterial activity against clinically sensitive as well as resistant strains of Mycobacterium tuberculosis. These derivatives are useful for the treatment of mycobacterial diseases, particularly those caused by pathogenic mycobacteria. The antimycobacterial activity of the compounds of the present invention is found to be superior to those of previously known compounds (Hudson, A; Imamura, T; Gutteride, W; Kanyok, T; Nunn, P. “The current anti-TB drug research and development pipeline” 2003; http://www.who.int/tdr/publications/publications/antitb_drug.htm). The present invention also relates to use of the novel compounds for treatment of latent tuberculosis including multi-drug resistant tuberculosis (MDR-TB). Multi drug-resistant tuberculosis (MDR-TB) is a strain of TB bacteria that has become resistant to at least two first-line anti-TB drugs.

The invention further relates to method of preparation of the novel compounds and pharmaceutical compositions containing the disclosed compounds under this invention.

### BACKGROUND OF THE INVENTION

Tuberculosis (TB) infection has become a worldwide problem, infecting in synergy with human immunodeficiency virus (HIV) infection (World Health Organization, Publication # WHO/TB/97.229). This contagious disease is transmitted through the air, and it is caused by the bacterium Mycobacterium tuberculosis, which can infect different organs of the human body. However, it most commonly affects the lungs, which is responsible for more than 75% of cases. It is estimated that 8.2 million of new TB cases occurred worldwide in the year 2000, with approximately 1.8 million deaths in the same year, and more than 95% of those were in developing countries (Corbett, E. L.; Watt, C. J.; Walker, N.; Maher, D.; Williams, B. G.; Raviglione, M. C.; Dye, C. Arch. Intern. Med., 2003, 1639, 1009). Two developments make the resurgence in TB especially alarming. The first is pathogenic synergy with HIV (Nakata, K.; Honda.; Tanaka, N.; Weiden, M.; and Keicho, N. Tuberculosis in patients with acquired immune deficiency syndrome. Kekkaku 2000, 75, 547-556). The overall incidence of TB in HIV-positive patients is 50 times that of the rate for HIV-negative individuals (Dye, C.; Scheele, S.; Dolin, P.; Pathania, V.; Raviglione, M. C. JAMA, 1999, 282, 677). The second is the emergence of drug-resistant and multi-drug-resistant TB (MDR-TB) (Basso, L. A.; Blanchard, J. S. Adv. Exp. Med. Biol., 1998, 456, 115). Drugs used for the treatment of tuberculosis involve the combination of multiple agents such as Isoniazid, Rifmapcin, Pyrazinamide, Ethambutol, Streptomycin, Para-amino salicylic acid, Ethionamide, Cycloserine, Capreomycin, Kanamycin, Ciprofloxacin, Ofloxacin, Thioacetazone etc (Basso, L. A.; Blanchard, J. S. Adv. Exp. Med. Biol. 1998, 456, 115). For example, the regimen recommended by the U.S. Public Health Service (http://www.hhs.gov/pharmacy/pp/DHHSpresent/) is a combination of Isoniazid, Rifampicin and Pyrazinamide for two months, followed by Isoniazid and Rifampicin alone for a further four months. These drugs are continued for another seven months in patients infected with HIV. For the treatment of multi-drug resistant tuberculosis streptomycin, kanamycin, amikacin, capreomycin, ethionamide, cycloserine, ciprofloxacin and ofloxacin are added to the combination therapies (World Health Organization, Anti-tuberculosis drug resistance in the world: Third Global Report, 2004). At present there is no single agent that can treat the tuberculosis as well as no combination that can shorten the duration of treatment.

The past decade has seen dramatic advances in our understanding of the metabolic and intracellular lifestyle of M. tuberculosis, culminating in the recent publication of its complete genomic DNA sequence (Cole, S. T. et al. Nature 1998, 393, 537-544). The emphasis of mycobacterial research has now shifted from gene hunting to interpretation of the biology of the whole organism in an effort to define the activities, which are likely to be critical for its survival and thus, amenable to the development of new drugs (Barry, C. E. et al. Biochemical Pharmacology 2000, 59, 221-231)

There is a great need to discover and develop entirely new class of agents possibly acting on completely novel targets through mechanism of actions different from those of existing drugs (O'Brien, R. J; Nunn, P. P. “The need for new drugs against tuberculosis” Am. J Respir. Crit. Care Med. 2001, 162, 1055-1058). They should have better tolerability (lower toxicity) than existing drugs, and have improved pharmacokinetic properties, in order to make intermittent chemotherapy feasible. Hence more effective and less toxic anti-tubercular agents are urgently needed to shorten the duration of current treatment, improve the treatment of MDR-TB, and to provide effective treatment of latent tuberculosis infection (Hingley-Wilson, S. M; Sambandamurthy, V. K; Jacobs J. “Survival perspectives from the world's most successful pathogen, M. tuberculosis” Nat. Immunol. 2003, 4, 949-955, WR). Several new classes of compounds have been synthesized and tested for monitoring the activity of M. tuberculosis, the details of the chemistry and biology of which could be found in a number of recent reviews: Hudson, A.; Imamura, T.; Gutteride, W.; Kanyok, T.; Nunn, P. “The current anti-TB drug research and development pipeline” 2003; http://www.who.int/tdr/publications/publications/antitb_drug.htm and “New small-molecule synthetic antimycobacterials” Antimicrobial agents and chemotherapy, 2005, 49, 2153-2163, and the references cited therein.

Substituted Quinoline derivatives constitute a class of compounds, which hold promise as antimycobacterial agents. The Quinoline derivatives which have been synthesized and tested for anti-tubercular activity and other non-tubercular activity have been disclosed by:
(a) Janssen pharmaceutica (WO2004/011436), this patent describes the inhibitory activity shown by various compounds, viz. R207910 (1) structure shown below, against M. tuberculosis, drug resistant mycobacteria and some non-tuberculosis mycobacteria.

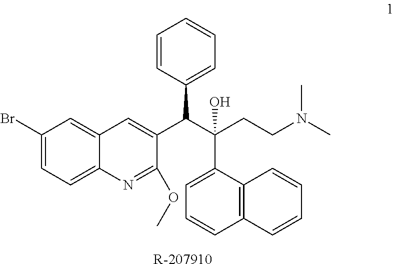

The MIC value (μg/mL) against the M. Tuberculosis strain (H37RV) exhibited by R207910 was 0.07 μg/mL.
(b) Some of the compounds described in the patent by Janssen pharmaceutica (WO2007/014885) have shown significant antimycobacterial activity against M. Tuberculosis. Most of the compounds can be represented by the general formula shown hereunder:

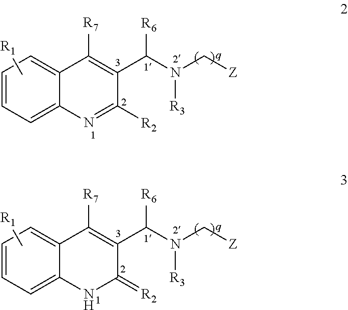

As per the generic structure of these compounds nitrogen (N2′) is fixed at the side chain C-3 that is always substituted with R₃ (CH₃, —CH(CH₃)₂, phenyl, substituted phenyl, benzyl, —(CH₂)₃N(CH₃)₂, and hetrocyles such as

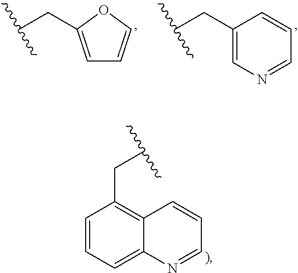

and a side chain of formula —(CH₂)_{q}—X—NR₄R₅, wherein, q is an intiger from 1, 2 or 3; X is CH₂ or —CO and R₄R₅ is an independent or together alkyl amine, heterocyclic amine or aromatic amine. On the basis of above description N2′ will always have a side chain of formula —(CH₂)_{q}—X—NR₄R₅ for that at least one —(CH₂)_{q}, if q=1 to satisfy the generic formula. The bond can be defined as —N—C—CO— or —N—C—CH₂—, and R₃ should be at least H, therefore it is chemically quite clear that N2′ cannot be part of a cyclic structure such as in imidazole, pyrazoles, aryl piperazines etc.

In view of this, we herein disclose our present invention of the novel antimycobacterial compounds, which have directly linked —C—N-Hetrocyclic amines, piperazines, substituted pyrazoles, ureas, carbodiimides etc; all the substitution and variables are explained in Table 1. The MIC values of these compounds against the M. Tuberculosis strain (H37RV), M. fortuitum, M. kansasii, and clinical isolates (MDR-TB strains) are found to be in range of 0.39 to 6.25 μg/mL.
(a) Janssen pharmaceutica (WO2007/014940) has reported the synthesis and antibacterial activity of several analogous of R207910, having the general formula 4 and 5 shown hereunder:

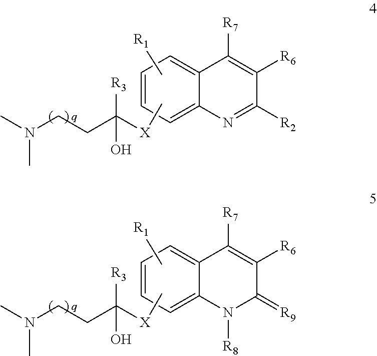

The IC₉₀ values (4-6 μg/mL) of these compounds were determined against various bacteria such as Bacillus subtilis, Escherichia coli, Enterococcus etc.
(b) Apart from that, substituted quinolines were already disclosed in U.S. Pat. No. 5,965,572 for treating antibiotic resistant infections, WO 00/34265, to inhibit the growth of bacterial microorganisms.
(c) WO 2005/070924, WO 2005/070430 and WO 2005/075428 describe the synthesis and antimycobacterial activity of substituted quinolines.

None of the above mentioned disclosures however report or suggest the antimycobacterial activity of Quinoline derivatives described in our present invention.

### OBJECTS OF THE INVENTION

The basic object of present invention is to meet the urgent demand that exists for novel antimycobacterial agent by the synthesis of novel Quinoline derivatives, which:

1. Show bactericidal activity against MDR and latent strains of M. tuberculosis
2. Act through novel mode of action,
3. Show reduced toxicity compared to the known anti-TB drugs,
4. Show improved bioavailability/reduce the amount of the drug to be taken, and
5. Decrease the overall treatment time.

### SUMMARY OF THE INVENTION

The present invention relates to novel Quinoline, non-quinoline (naphthalene) and their conformationally-constrained derivatives according to formula I, II, III, IV, V, VI, VII, VIII, IX and X (FIG. 1)

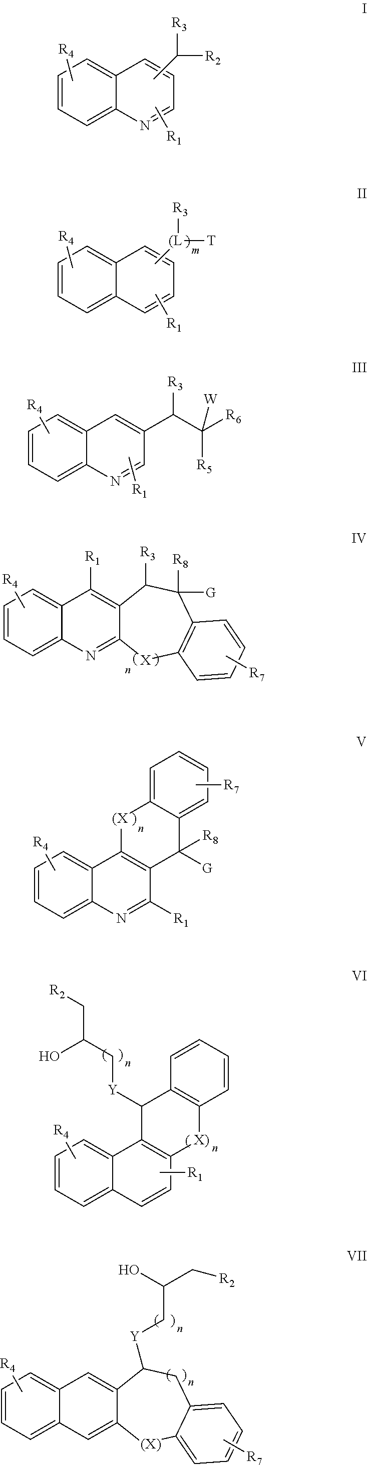

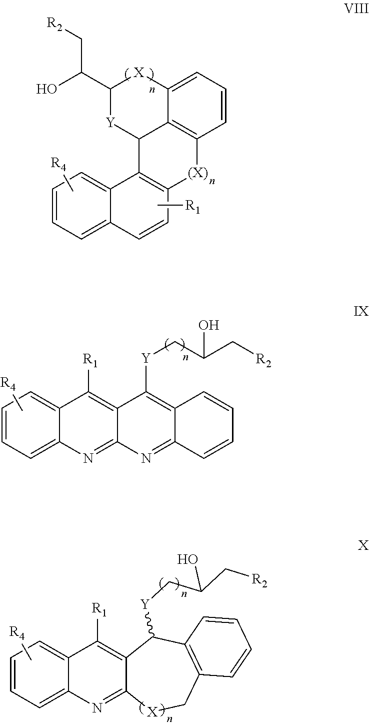

the pharmaceutically acceptable acid or base salts thereof, the stereochemically isomeric forms thereof, the tautomeric forms thereof and N-oxide forms thereof, wherein all the chemical variations are described in Table 1.

TABLE 1
Substitution patterns and Variables, and their Chemical Descriptions as designated in the general
formulae I-X (Fig. 1)

| Substitution | | |
|---|---|---|
| and Variables | | Chemical Description |
| L | | C, CH or a hetero atom from N, O or S |
| m | | Is an integer 0 to 4 |
| n | | Is an integer 0 to 2 |
| W | | H, OH, COOH, CN, alkoxy |
| R₁ | | Hydrogen, halo, halo alkyl, acyl, cyno, hydroxy, aminoalyl, Het, |
| | | Heterocyclic amines i.e pyrolidinyl, pyrrolyl, pyrrolinyl, |
| | | imidazolidinyl, imidazolyl, pyrazolyl, triazolyl, tetrazolyl, piperidinyl, |
| | | pyridinyl, pyridazinyl, pyrimidinyl, pyrazinyl, trizinyl, morpholinyl |
| | | and thiomorpholinyl, alkyloxy, thio, alkylthio, alkyloxyalkyloxy, |
| | | trifluoroalkyl, trifluoroalkylalkoxy, alkylthioalkyl mono or |
| | | dialkylamino or a radical formula 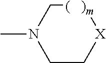 |
| | X | C═O, CH₂, O, S, SO, SO₂, NH, N-alkyl or N-aryl of formula |
| | | 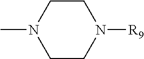 |
| | R₉ | Wherein, R₉ is phenyl which is unsubstituted or substituted with 1-2 |
| | | substituents each independently selected from the group consisting of |
| | | halogen, C₁-C₄ alkyl, C₁-C₄ alkoxy, acyl, cyano, C₁-C₄ thioalkoxy, |
| | | nitro, amino, haloalkyl, haloalkoxy etc.; unsubstituted or substituted |
| | | benzyl; unsubstituted or substituted heteroaryl; unsubstitutecd or |
| | | substituted heteroaroyl or unsubstituted or substituted diphenyl |
| | | methyl, unsubstituted or substituted naphthyl |
| R₂ | | Is selected from the group of pyrolidinyl pyrrolyl, pyrrolinyl, |
| | | imidazolidinyl, imidazolyl, pyrazolyl, triazolyl, tetrazolyl, piperidinyl, |
| | | pyridinyl, pyridazinyl, pyrimidinyl, pyrazinyl, trizinyl, morpholinyl |
| | | and thiomorpholinyl, optionally substituted with alkyl, haloalkyl, |
| | | hydroxy, alkoxy, amino, mono- or dialkylamino, acyl, nitro, cyano, |
| | | alkylthio, alkyloxyalkyl, alkylthioalkyl, pyrimidinyl and substituted |
| | | piperazinc, unsubstituted or substituted pyrazoles that can be |
| | | represented with FIG. 2. |
| | | 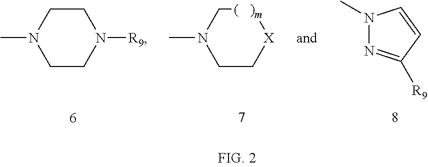 |
| | | R₉, m and X as explained for R₁ |
| T | | Is described by 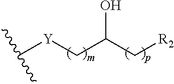 |
| | | Wherein: |
| | P | Is an integer from 0-4 |
| | Y | Is a heteroatom from the group of N, O, S |
| | | m and R₂ are as explained above in this Table. |
| R₃ | | Is phenyl or substituted phenyl, aryl or unsubstituted or substituted |
| | | heteroaryl, unsubstituted or substituted naphthyl etc. |
| R₄ | | Is hydrogen, halo, halo alkyl, cyno, hydroxy, acyl, nitro, Ar, alkyl, |
| and | | Het, alkyloxy, thio, alkylthio, alkyloxyalkyloxy, alkylthioalkyl mono |
| R₇ | | or dialkylamino or pyrrolidinyl pyrrolyl, pyrrolinyl, imidazolidinyl, |
| | | imidazolyl, pyrazolyl, triazolyl, tetrazolyl, piperidinyl, pyridinyl, |
| | | pyridazinyl, pyrimidinyl, pyrazinyl, trizinyl, morpholinyl and |
| | | thiomorpholinyl, optionally substituted with alkyl, haloalkyl, hydroxy, |
| | | alkoxy, amino, mono- or dialkylamino, acyl, nitro, cyano, alkylthio, |
| | | alkyloxyalkyl, alkylthioalkyl, pyrimidinyl and substitute dpiperazine, |
| | | unsubstituted or substituted pyrazoles as per FIG. 2. Unsubstituted |
| | | and substituted guanidine derivatives, ureas and thio ureas and |
| | | carbodiimides as per FIG. 3. |
| | | 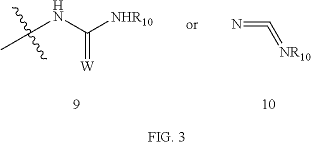 |
| | | Wherein, |
| | | W is O, S, NH |
| | | R₁₀ is H, Substituted or unsubstituted aryl, alkyl etc. |
| R₅ | | When one of R₅ and R₆ is 11, the other is 12 and R₁₁, R₁₂ are selected |
| and | | from the groups: |
| R₆ | | 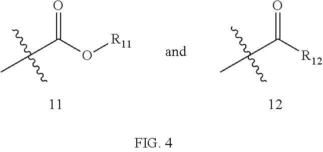 |
| | R₁₁ | Wherein, R₁₁ hydrogen, phenyl that is substituted or unsubstituted |
| | | with 1-2 substituents each independently selected from the group |
| | | consisting of halogen, C₁-C₁₂ alkyl; |
| | R₁₂ | R₁₂ is hydrogen, halo, halo alkyl, cyno, hydroxy, Ar, alkyl, Het, |
| | | alkyloxy, thio, alkylthio, alkyloxyalkyloxy, alkylthioalkyl mono or |
| | | dialkylamino or pyrrolidinyl pyrrolyl, pyrrolinyl, imidazolidinyl, |
| | | imidazolyl, pyrazolyl, triazolyl, tetrazolyl, piperidinyl, pyridinyl, |
| | | pyridazinyl, pyrimidinyl, pyrazinyl, trizinyl, morpholinyl and |
| | | thiomorpholinyl, optionally substituted with alkyl, haloalkyl, hydroxy, |
| | | alkoxy, amino, mono- or dialkylamino, acyl, nitro, cyano, alkylthio, |
| | | alkyloxyalkyl, alkylthioalkyl, pyrimidinyl and substituted piperazine, |
| | | unsubstituted or substituted pyrazoles as per FIG. 2. |
| R₈ | | When R₈ is hydrogen, halo, halo alkyl, cyno, hydroxy, Ar, alkyl, acyl, |
| | | Het, alkyloxy, thio, alkylthio, alkyloxyalkyloxy, alkylthioalkyl mono |
| | | or dialkylamino or pyrrolidinyl pyrrolyl, pyrrolidinyl, imidazolidinyl, |
| | | imidazolyl, pyrazolyl, triazolyl, tetrazolyl, piperidinyl, pyridinyl, |
| | | pyridazinyl, pyrimidinyl, pyrazinyl, trizinyl, morplinyl and |
| | | thiomorphlinyl, optionally substituted with alkyl, haloalkyl, hydroxy, |
| | | alkoxy, amino, mono- or dialkylamino, acyl nitro, cyano alkylthio, |
| | | alkyloxyalkyl, alkylthioalkyl, pyrimidinyl and substituted piperazine, |
| | | unsubstituted or substituted pyrazoles as per FIG. 2 then G is from |
| | | subgroup G₁, G₂, G₃, G₄, G₅ and G₆. |
| G | | Is a group of different functionality, holds subgroup G₁, G₂, G₃, G₄, |
| | | G₅ and G₆. These subgroups are shown below: |
| | G₁ | When R₈ ≠ H then G = N—O—R₁₃, or G = NH₂, |
| | | R₁₃ is H, alkyl, aryl, substituted aryl, acyl, N, N dimethyl carbamoyl, |
| | | hydrolysable esters, bioesters, phosphonate esters, acyl esters, amino |
| | | acyl esters (eg. of hydrophilic and hydrophobic esters), long chain |
| | | hydroxy fatty acids, hydroxy acids (eg. Citric acid), sugar acids (such |
| | | as gluconic acid), sugars like ribose, arabinose, allose, xylose, aldose, |
| | | pyranose, furanose, etc. of formula: |
| | | 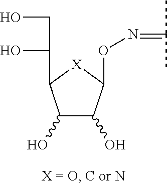 |
| | G₂ | When R₈ = H then G = R₂ and not limited to Pyrolidinyl, pyrrolyl, |
| | | pyrrolinyl, imidazolidinyl, imidazolyl, pyrazolyl, triazolyl, tetrazolyl, |
| | | piperidinyl, pyridinyl, pyridazinyl, pyrimidinyl, pyrazinyl, trizinyl, |
| | | morpholinyl and thiomorpholinyl, optionally substituted with alkyl, |
| | | haloalkyl, hydroxy, alkoxy, amino, mono- or dialkylamino, acyl, nitro, |
| | | cyano, alkylthio, alkyloxyalkyl, alkylthioalkyl, pyrimidinyl and |
| | | substituted piperazine, unsubstituted or substituted pyrazoles (as per |
| | | FIG. 2), substituted or unsubstituted guanidine derivatives, ureas |
| | | and thioureas, substituted and unsubstantiated carbodiimides as per |
| | | FIG. 3. |
| | G₃ | When R₈ = H, then G can be represented with formula: |
| | | 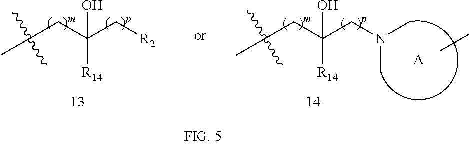 |
| | R₁₄ | R₁₄ Hydrogen, Alkyl substituted or unsubstituted aryl, htereo aryl, |
| | | naphthyl etc. |
| | | m and p are integers 0 to 4 |
| | | R₂ is described above in this table. |
| | | Where in ring A (FIG. 5) is hetrocyclyl, wherein if said |
| | | hetrocyclyl contains an NH moiety that nitrogen may be optionally |
| | | substituted by a group selected from C₁₋₄ alkyl, C₁₋₄ alkanoyl, C₁₋₄ |
| | | alkylsulphonyl, C₁₋₄ alkoxy carbonyl, carbamoyl, N— (C₁₋₄ alkyl) |
| | | carbamoyl, N,N— (C₁₋₄ alkyl) carbamoyl, benzyl, benzyloxycarbonyl, |
| | | benozyl and phenyl sulphonyl. |
| | G₄ | When R₈ = CH₃, G= OR₁₃ |
| | | 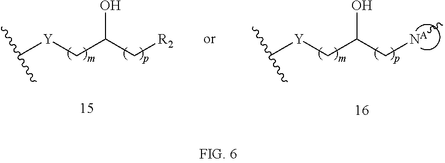 |
| | | R₂ , R₁₄, m, p and other chemical variations are same as for G₃ |
| | | Y is same as explained for R₃. |
| | | R₁₃ = Same as defined in G₁ |
| | G₅ | When R₈ = OR₁₅ then G will be |
| | | 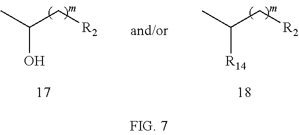 |
| | R₁₅ | Alkyl, substituted or unsubstituted aryl, hetero aryl, naphthyl etc. |
| | | R₂, R₁₄, m, p and other chemical variations are same as in G₃ |
| | G₆ | 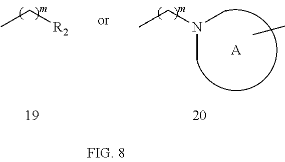 |
| | | When R₈ is |
| | | Then G is expressed with formula 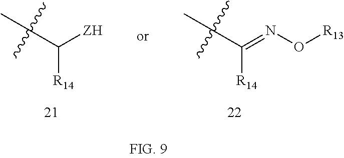 |
| | | R₂, R₁₃, R₁₄, m and other chemical variations are same as in G₃ |
| | | Z is O, S, NH. |

Another aspect of present invention provides methods for synthesis of compound of formula I, II, III, IV, V, VI, VII, VIII, IX and X their tautomers, enantiomers, diastereomers, N-Oxides, Polymorphs and pharmaceutical acceptable salts, hydrolysable esters/ethers thereof comprising of compounds of formulae 23-29 (FIG. 10):

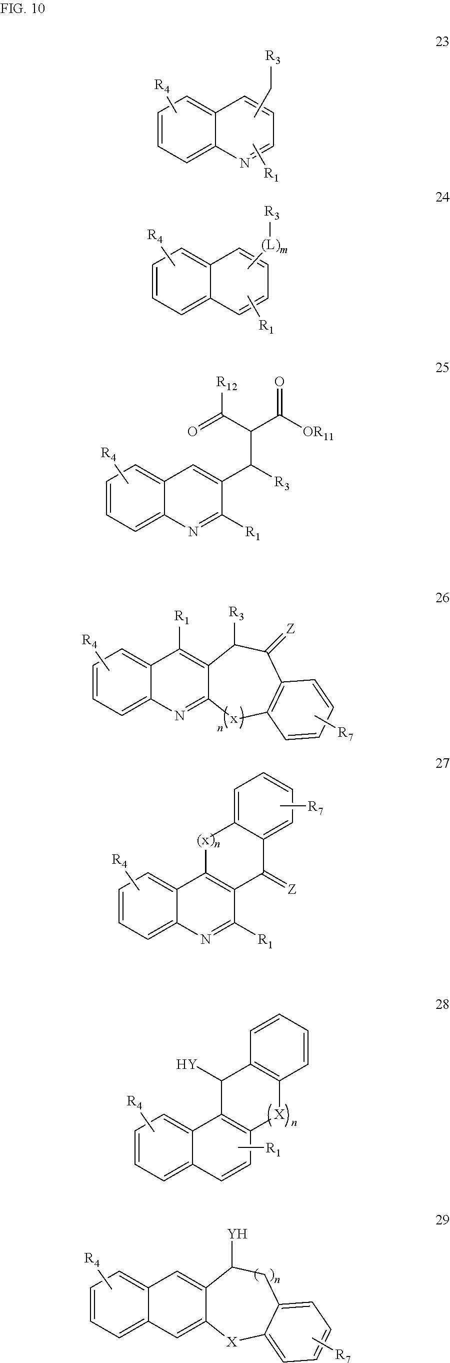

FIG. 10: (R₁, R₃, R₄, R₇, R₁₁, R₁₂, L, X, Z, m and n are described in Table 1)

The present invention provides pharmaceutical compositions useful in the treatment of microbial conditions such as tuberculosis including multidrug resistant tuberculosis comprising of (a) at least one of the compounds of formula I, II, III, IV, V, VI, VII, VIII, IX and X its tautomers, enantiomers, diastereomers, N-oxides, polymorphs and pharmaceutically acceptable salts, and (b) pharmaceutically acceptable additives.

In yet another aspect, the present invention provides a method of inhibiting the microbial cell/conditions with the compounds of formula I, II, III, IV, V, VI, VII, VIII, IX or X disclosed in present invention, its tautomers, enantiomers, diastereomers, N-oxides, polymorphs and pharmaceutically acceptable salts with or without carriers. The microbial cell/conditions tested with our compounds are those of Mycobacterium tuberculosis, drug-resistant Mycobacterium tuberculosis, Mycobacterium kansasii, Mycobacterium fortuitum or Mycobacterium-intracellular complex.

### DETAILED DESCRIPTION OF THE INVENTION

In the framework of this application Alkyl, Ar, Het, Halo, haloalkyl are defined as below and the other substitutions, chemical variations are described in Table 1.
Alkyl is a straight or branched saturated or unsaturated hydrocarbon radical having from 1-32 carbon atoms; or is a cyclic saturated hydrocarbon radical; or is a saturated hydrocarbon radical attached to a straight or branched saturated hydrocarbon; wherein each carbon atom can be optionally substituted with halo, hydroxy, alkyloxy or oxo;
Ar is homocycle selected from the group of phenyl, naphthyl each optionally substituted with 1, 2 or 3 substituents, each substituent independently selected from but not limited to hydroxy, halo, cyno, nitro, amino, mono-di-aminoalkyl, halo alky, alkyl haloalkoxy, alkoxy, carboxyl, alkyloxy carbonyl, amino carbonyl, morpholinyl;
Het is any heterocyclic ring systems containing one or more heteroatoms (either N, O and/or S), but not limited to pyrrolidinyl pyrrolyl, pyrrolinyl, imidazolidinyl, imidazolyl, pyrazolyl, triazolyl, tetrazolyl, piperidinyl, pyridinyl, pyridazinyl, pyrimidinyl, pyrazinyl, trizinyl, morpholinyl and thiomorpholinyl; or a bicyclic heterocycle selected from the group of quinolinyl, quinoxalinyl, indolyl, benzimidazolyl, benzoxazolyl, benzisoxazolyl, benzthiazolyl, benzisothiazolyl, benzofuranyl, benzothienyl: each monocyclic and bicyclic hetrocycle may optionally substituted on a carbon atom with 1, 2, 3 substituents selected from the group of halo, hydroxy, alkyl, nitro, cyano, acyl, sulfonyl. sulfinyl or alkoxy;
Halo is a substituent at any system selected from the group: fluoro, chloro, bromo and iodo;
Haloalkyl is a straight or branched saturated or unsaturated hydrocarbon radical having from 1-32 carbon atoms; or is a cyclic saturated hydrocarbon radical; or is a saturated hydrocarbon radical attached to a straight or branched saturated hydrocarbon; wherein one or more carbon atom(s) are substituted with one or more halo atoms as described above.

Preferably, the present invention relates to compounds of formula I, II, III, IV, V, VI, VII, VIII, IX, X and their analogs. Another aspect of present invention provides methods for synthesis of compound of formula I, II, III, IV, V, VI, VII, VIII, IX and X their tautomers, enantiomers, diastereomers, N-Oxides, Polymorphs and pharmaceutically acceptable salts thereof comprising reacting of compounds of described in FIG. 10, all substitutions and variables for which are described in Table 1.

Furthermore, the compounds of formula I, II, III, IV, V, VI, VII, VIII, IX and X of this invention includes the pharmaceutically acceptable acid addition salts are defined to comprise the therapeutically active non-toxic acid addition salts formed with organic and inorganic acids by methods well known in art. These salts may be used in place of free bases. Acid addition salts may be obtained by treating the base form of disclosed compounds with appropriate acids such as malic acid, fumaric acid, benzoic acid, ascorbic acid, acetic acid, hydroxy acetic acid, propanoic acid, lactic acid, pyruvic acid, oxalic acid, malonic acid, succinic acid, malic acid, tartaric acid, citric acid, methanesulphonic acid, ethanesulphonic acid, benzenesulphonic acid, p-toluenesulphonic acid, salicylic acid, gluconic acid, aspartic acid, palmitic acid, itaconic acid, glycolic acid, hydrochloric acid, hydrobromic acid, sulfuric acid, nitric acid and phosphoric acid and the like.

The present invention also includes all stereochemically isomeric forms that the compounds of either formula may possess. More in particular, stereogenic centers may have the R- or S-configuration; substituents on bivalent cyclic (partially) saturated radicals may have either E or Z configuration.

The present invention also provides the pharmaceutical compositions containing compound of formula I, II, III, IV, V, VI, VII, VIII, IX or X for the treatment of Mycobacterium tuberculosis. These compositions comprises an effective concentration of compound of formula I, II, III, IV, V, VI, VII, VIII, IX or X its tautomers, enantiomers, diastereomers, N-oxides, pharmaceutically acceptable salts or polymorphic forms thereof, in combination with a pharmaceutically acceptable carrier and optionally in the presence of excipients.

Further, the present invention also relates to the use of a compound of either formula I, II, III, IV, V, VI, VII, VIII, IX or X the pharmaceutically acceptable acid salts, thereof and the various possible tautomers, enantiomers, diastereomers, N-oxides, polymorphs thereof, as well as any of the aforementioned pharmaceutical composition thereof for the treatment of mycobacterial conditions such as Mycobacterium tuberculosis, Mycobacterium avium-intracellular complex, drug-resistant Mycobacterium tuberculosis, Mycobacterium fortuitum or Mycobacterium kansasii.

In a further embodiment the compound of either formula I, II, III, IV, V, VI, VII, VIII, IX or X the pharmaceutically acceptable salts, thereof also exhibit utility as antimalarial, antiprotozoal (Leishmania amazonensis, Trypanosoma cruzi), antifungal (Candida albicans, Candida tropicalis, Candida krusei, Cryptococcus neoformans, Aspergillus niger), antibacterial (Staphylococcus aureus, Streptococci pneumonia, Pseudomonas aeruginosa, Klebsiella pneumonia), antiviral (HIV, Herpes simplex virus) and antitumor agents.

### Section 1

### General Preparation

The compound disclosed in present invention can be synthesized by executing the described steps by any skilled person knowledgeable in the current state-of-the-art in the chemical synthesis.

The compounds covered by formula I (eg. 31) can be synthesized by reactant of formula 30 with any compound of formulas 6, 7 or 8 as per the Scheme 1.

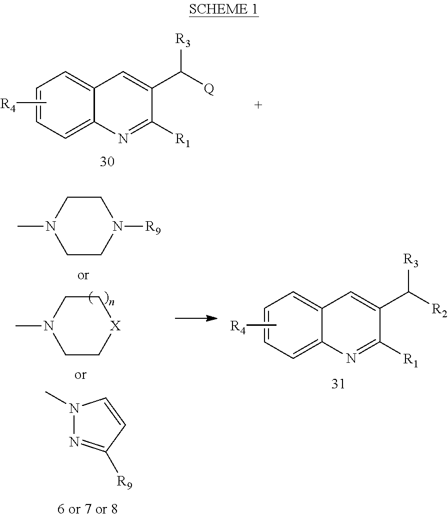

Appropriate compound of formula 6 or 7 or 8 treated with compound of formula 30 in the presence of suitable base and aprotic solvent, wherein all variable reaction conditions can be suitably included. The preferable reaction temperature can within the range of 25° C. to 120° C. The starting material and the required intermediates for the synthesis of 30 and 6 or 7 or 8 are either commercially available or may be prepared according to the literature procedures generally known in the art.

The required intermediate of formula 30 can be prepared as per the reaction described in Schemes 2 and 3:

For the preparation of compound of formula 30, Baylis-Hillman chemistry (Pathak, R.; Madapa, S.; Batra, S. Tetrahedron 2007, 63, 451-460) can be exploited as per the procedure described in Scheme 2. In step 1, Baylis-Hillman adduct, which was prepared by DABCO promoted Baylis-Hillman reaction from benzaldehyde (Bouzide, A. Org. Lett. 2002, 4, 1347-1350), was treated with appropriate acetylating agent in the presence of organic base and suitable chlorinated solvent (Ramachandran, P. V.; Burghardt, T. E.; Rama Reddy, M. V. Tetrahedron Lett. 2005, 46, 2121-2124). The reaction may be carried out ranging from room to reflux temperature. In the next step, nucleophilic substitution of suitable derivative of aniline in the presence of suitable base such as DABCO at one of the variable reaction conditions was carried out. In the step 3, adduct obtained in step 2 is treated with appropriate acid such as trifluoroacetic acid, polyphoshphoric acid or POCl₃ with or without surfactant at any of the variable range of temperature (60° C.—reflux temperature) led to the product, to be used in the next step. In next step 4, the adduct obtained from step 3 was treated with appropriate base such potassium carbonate and suitable solvent like acetone at variable range of temperature, such as room temperature to reflux temperature. In next step 5, isomerized adduct obtained in step 4 was treated with POCl₃ in presence of a suitable solvent such as toluene. This reaction may conveniently be carried out at a temperature ranging between room temperature to reflux temperature. In the next step 6, specific R₁ group is introduced to the adduct obtained in step 5 under a suitable reaction condition. In the next step 7, adduct obtained in step 6 was treated with one of the suitable reagents to introduce the more labile group. The preferably reagent is N-Bromo succinamide and a radical generator such as benzoyl peroxide in a suitable solvent and reaction condition.

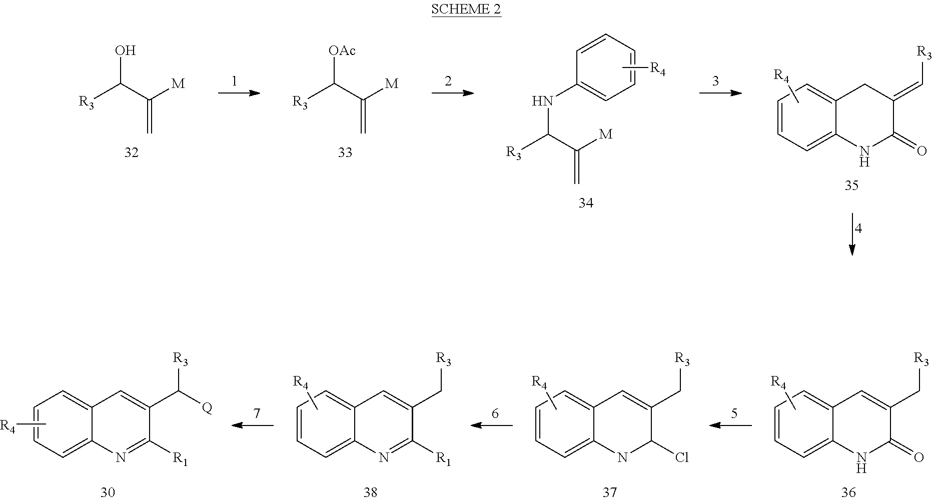

An alternative synthetic route for the preparation of compound of formula 30 is described in Scheme 3.

In this strategy, appropriate aniline is reacted with suitable acyl chloride such as hydrocinamoyl chloride in the presence of suitable base and a suitable solvent at temperature range between room to reflux temperature. In the step 2, adduct obtained in step 1 is treated with phosphoryl chloride in the presence of N,N-dimethyl formamide (formylation followed by cyclization). The reaction may conveniently be carried out at temperature ranging from room temperature to reflux temperature. In the step 3, specific R₁ group is introduced to the product obtained in step 2 under suitable reaction conditions. In the next step 4, adduct obtained in step 3 was treated with various reagents to introduced the more labile group preferably the reagent is N-Bromo succinamide and radical generator benzoyl peroxide in a suitable solvent and reaction condition.

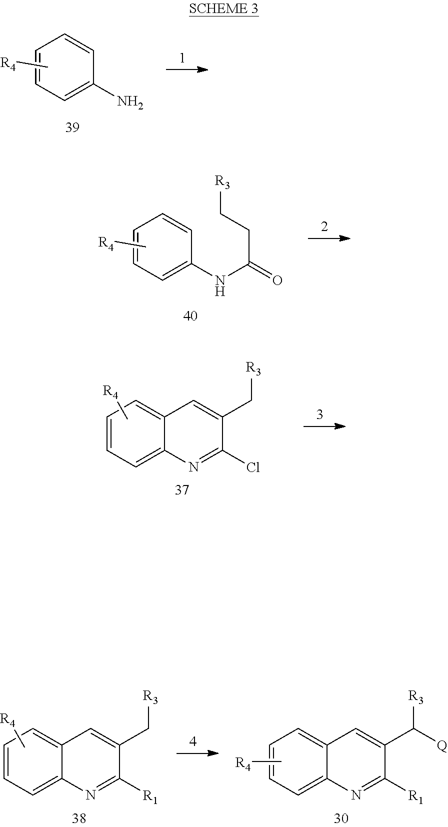

For the preparation of compounds covered under general formula II, Scheme 4 can be followed. Compounds with structure 41 could be easily converted to the corresponding chloride 42 by treatment with a suitable chlorinating agent such as thionyl chloride or POCl₃ at temperature ranging from room temperature to reflux. Friedal Craft reaction of 42 with a suitable aromatic compound at temperature ranging from room temperature to reflux gave compounds with structure 43, which upon reduction with a suitable reducing agent like sodium borohydride or lithium aluminum hydride followed by reaction with a compound like epi-chlorohydrin gave epoxide 45. Opening of epoxides in 45 with different nucleophiles gave the compounds with generic structure II.

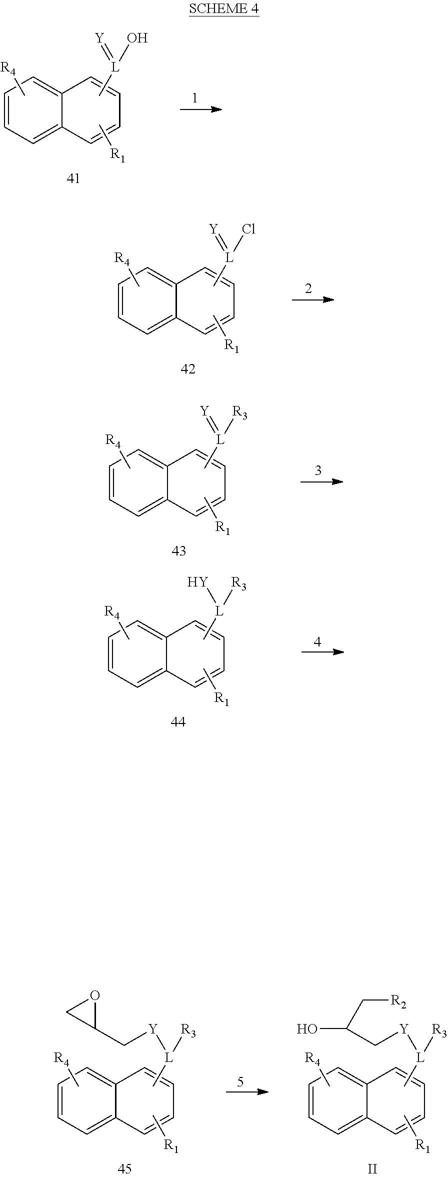

Compounds of general formula III may be prepared according to Schemes 5 and 6

Compounds of formula 30 (Q is a suitable leaving group) and 46 may be reacted together in presence of suitable base for example sodium hydride, in a suitable solvent for example toluene or tetrahydrofuran.

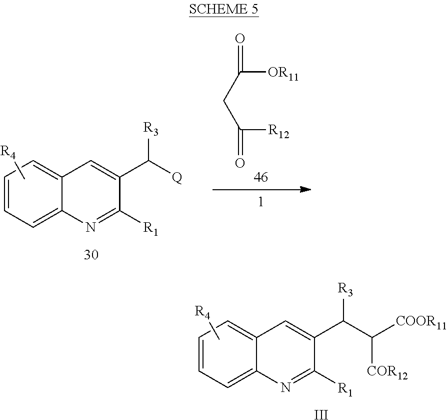

Intermediate 46 can be prepared according to Scheme 6

Reaction Scheme described in Scheme 6 comprises step 1 in which an appropriate diester for example diethyl malonate is selectively hydrolyzed under suitable reaction condition, for example, in 1N aqueous solution of NaOH in appropriate solvent like ethanol. The reaction can be carried out at a temperature ranging from room to reflux temperature. In the step 2, monoacid obtained in step 1 is reacted with appropriate amines in presence of suitable coupling reagent (standard peptide coupling reagents known in the art can be employed as suitable coupling reagents for example dicyclohexyl carbodiimide, carbodiimdazole or EDC with or without additive) in a suitable solvent, for example, dichloromethane, tetrahydrofuran or diethyl ether.

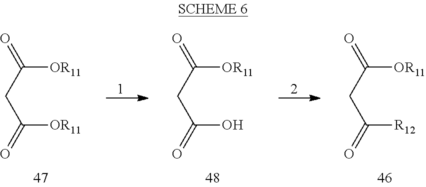

Another alternative synthetic approach can be employed for the preparation of compound of formula III is shown in Scheme 7

Compound 30 and an appropriate diester may be reacted together in presence of a suitable base, for example, sodium hydride, in a suitable solvent, for example, toluene or tetrhydrofuran. The reaction can be carried out at any specific temperature ranging from room to reflux temperature. In the step 2, adduct obtained in step 1 is treated with 1N aqueous solution of NaOH in an appropriate solvent such as ethanol. The reaction may conveniently be carried out at any temperature ranging from room to reflux temperature. In the step 3, monoacid obtained in step 2 is reacted with appropriate amines in presence of suitable coupling reagent (any of the standard peptide coupling reagents known in the art can be employed as suitable coupling reagents, for example, dicyclohexyl carbodiimide, carbodiimdazole or EDC with or without additive) in a suitable solvent, for example, dichloromethane, tetrahydrofuran, N,N-dimethyl formamide or diethyl ether. The reaction may conveniently be carried out at temperature ranging from room to reflux temperature,

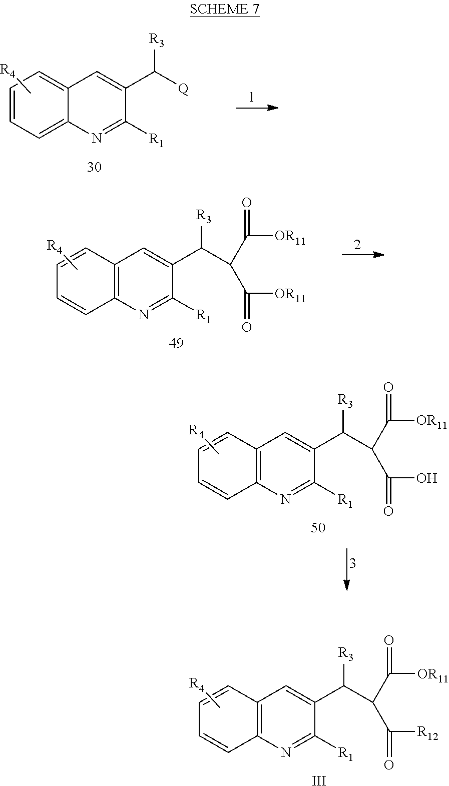

### EXPERIMENTAL

### Part-One

Representative examples of methods for the preparation of compounds reported in this invention are described below.

Preparation of the Intermediate Compounds:

### Method A

### Preparation of ethyl 2-(Hydroxy-phenyl-methyl)-acrylic acid ethyl ester

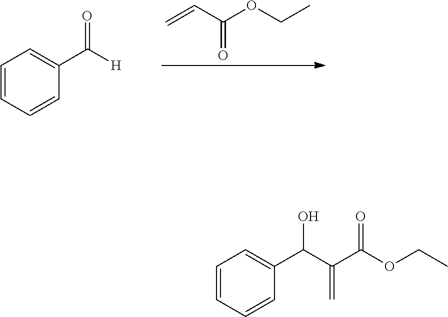

A mixture of benzaldehyde (13.8 g, 130.0 mmol), ethyl acrylate (10.0 g, 100.0 mmol) and 1,4-diazabicyclo [2.2.2] octane (DABCO, 2.24 g, 20.0 mmol) was stirred for 5 days at rt. The mixture was diluted with ethyl acetate (300 mL), washed with 1 M aqueous solution of hydrochloric acid (2×100 mL), the organic extract was dried over anhydrous sodium sulfate, filtered and the solvent was evaporated to obtain a sticky mass. Purification by column chromatography (silica gel 100-200 mesh, gradual elution, n-hexane to 5% ethyl acetate in n-hexane) gave ethyl 2-(Hydroxy-phenyl-methyl)-acrylic acid ethyl ester (13.0 g, 82%) as colorless oil. ¹H NMR (400 MHz, CDCl₃): δ 1.29 (t, J=7.0 Hz, 3H), 3.12 (br s, 1H, D₂O exchangeable), 4.19 (q, J=7.0 Hz, 2H), 5.53 (s, 1H), 5.86 (s, 1H), 6.27 (s, 1H), 7.24-7.42 (m, 5H).

### Preparation of ethyl 2-(acetoxy (phenyl) methyl) acrylate

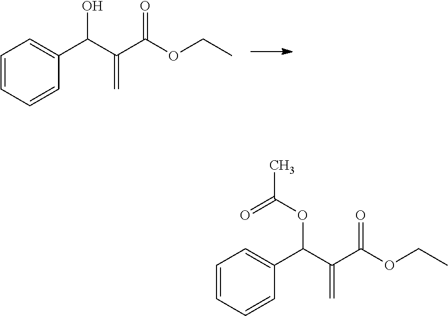

To a cooled (0° C., ice bath) dichloromethane (50 mL) solution of 2-(Hydroxy-phenyl-methyl)-acrylic acid ethyl ester (10.0 g, 48.5 mmol), anhydrous pyridine (5 mL) and acetyl chloride (19.0 g, 242.0 mmol) were added and the mixture was stirred at 0° C. for 1 h. The reaction was diluted with dichloromethane (100 mL), washed with 1 M aqueous solution of hydrochloric acid (2×50 mL), water (2×50 mL) and brine (50 mL). The organic extract was dried over anhydrous sodium sulfate, filtered and solvents were evaporated under reduced pressure to obtain ethyl 2-(acetoxy (phenyl) methyl) acrylate (11.3 g, 94%) as oil, which was used for the next step without further purification and characterization.

### Preparation of ethyl 2-((4-bromophenylamino)(phenyl) methyl) acrylate

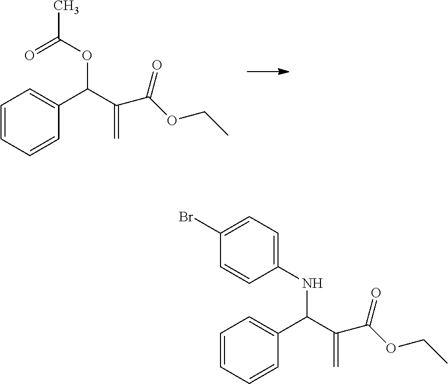

To the stirred solution of 2-(Acetoxy-phenyl-methyl)-acrylic acid ethyl ester (2.0 g, 8.0 mmol) in tetrahydrofuran-water (1:1, v/v, 20 mL) was added 1,4-diazabicyclo [2.2.2] octane (DABCO, 1.35 g, 12.0 mmol) at room temperature. After 15 min, 4-bromoaniline (1.65 g, 9.6 mmol) was added to the reaction, and stirred for 3 h. The solvent was evaporated under reduced pressure, the residue was extracted with ethyl acetate (3×100 mL), washed with water (2×50 mL) followed by brine (1×50 mL), dried over anhydrous sodium sulfate, filtered and the solvent was evaporated to obtain the crude product, which on purification by column chromatography (silica gel 100-200 mesh, eluent 10% ethyl acetate in n-hexane) gave pure 2-[(4-Bromo-phenylamino)-phenyl-methyl]-acrylic acid ethyl ester (3.0 g, 75%) as a thick brown oil. ¹H NMR (300 MHz, CDCl₃): δ 1.24 (t, J=7.1 Hz, 3H), 4.19 (q, J=7.1 Hz, 2H), 5.39 (s, 1H), 5.93 (s, 1H), 6.41 (s, 1H), 6.46-6.51 (m, 2H), 7.22-7.26 (m, 3H), 7.27-7.32 (m, 4H). [M+H]⁺=360, 362.

### Preparation of (E)-3-benzylidene-6-bromo-3,4-dihydroquinolin-2 (1H)-one

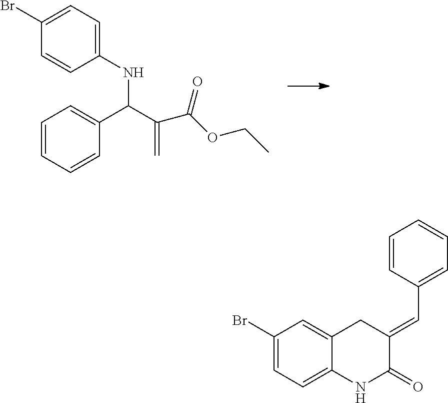

Trifluoroacetic acid (7 mL) was added to 2-[(4-Bromo-phenylamino)-phenyl-methyl]-acrylic acid ethyl ester (1.8 g, 5.0 mmol) and the mixture was refluxed for 12 hrs. The reaction mixture was poured into ice-water, neutralized with saturated sodium bicarbonate solution, the suspension formed was filtered, washed with ethyl acetate and dried under reduced pressure to afford 3-Bebzylidine-6-bromo-3,4-dihydro-1H-quinolin-2-one (1.21 g, 77%) as a white solid, Mp 220-222° C. ¹H NMR (300 MHz, DMSO-d₆): δ 3.82 (s, 2H), 7.15-7.28 (m, 5H), 5.52-7.56 (m, 1H), 7.63 (s, 1H), 7.79 (d, J=1.7 Hz, 1H). [M+H]⁺=315, 317.

### Preparation of 3-benzyl-6-bromoquinolin-2 (1H)-one

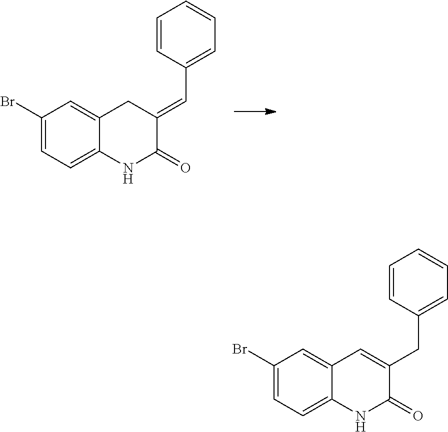

Activated potassium carbonate (0.90 g, 6.4 mmol) was added to a solution of 3-Benzylidine-6-bromo-3,4-dihydro-1H-quinolin-2-one (0.95 g, 3.0 mmol) in acetone (10 mL), and the mixture was refluxed for 15-20 min. The acetone was removed under reduced pressure, the residue was diluted with water, the suspension formed was filtered and dried under reduced pressure to afford 3-Benzyl-6-bromo-1H-quinoline-2-one (0.9 g, 95%) as a white solid, Mp 263° C. ¹H NMR (300 MHz, DMSO-d₆): δ 3.82 (s, 2H), 7.18-7.28 (m, 6H), 7.54-7.57 (m, 1H), 7.66 (s, 1H), 7.81 (d, J=2.1 Hz, 1H).

### Preparation of 3-benzyl-6-bromo-2-chloroquinoline

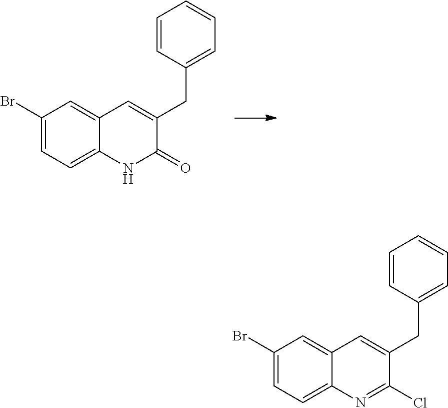

3-Benzyl-6-bromo-1H-quinoline-2-one (0.87 g, 2.8 mmol) and freshly distilled phosphorous oxychloride (5 mL) were refluxed together for 30 min. The reaction was poured into ice-water mixture, basified with saturated sodium bicarbonate solution to pH 8-8.5 and extracted with ethyl acetate (3×50 mL). The organic fractions were combined, washed with brine (50 mL), dried over anhydrous sodium sulfate, filtered and the solvents were evaporated under reduced pressure to obtain the crude product as a gum, which on purification by column chromatography (silica gel 100-200 mesh, eluent 3% ethyl acetate in n-hexane) gave pure 3-Benzyl-6-bromo-2-chloro-quinolin (0.85 g, 92%), Mp 102-104° C. ¹H NMR (400 MHz, CDCl₃): δ 4.22 (s, 2H), 7.20-7.24 (m, 2H), 7.26-7.31 (m, 1H), 7.32-7.38 (m, 2H), 7.65 (s, 1H), 7.72 (dd, J=12.0, 4.0 Hz, 1H), 7.84-7.88 (m, 2H). [M+H]⁺=332, 335.

### Preparation of 1-[2-(3-Benzyl-6-bromo-quinolin-2-yloxy)-5-fluoro-phenyl]-ethanone

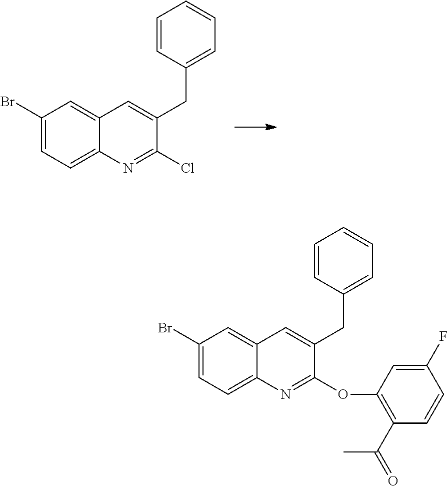

A mixture of 1-(2-Hydroxy-phenyl)-ethanone] (0.23 g, 1.51 mmol) and potassium carbonate (0.23 g, 1.70 mmol) in anhydrous dimethylsulfoxide (6 mL) was heated to 130° C. for 12 h under inert atmosphere. The mixture was poured into ice-water mixture, extracted with ethylacetate, washed with brine, dried over anhydrous sodium sulfate, filtered and concentrated under reduced pressure. Purification by column chromatography (silica gel 100-200 mesh, eluting with 8% ethyl acetate in n-hexane) gave pure 1-[2-(3-Benzyl-6-bromo-quinolin-2-yloxy)-5-fluoro-phenyl]-ethanone (0.28 g, 51.5%) as a pale yellow solid, Mp 115-117° C. ¹H NMR (400 MHz, CDCl₃): δ 2.23 (s, 3H), 4.22 (s, 2H), 6.98 (dd, J=9.2, 4.4 Hz, 1H), 7.18-7.23 (m, 1H), 7.26-7.37 (m, 5H), 7.48 (d, J=8.8 Hz, 1H), 7.52 (dd, J=8.8, 3.2 Hz, 1H), 7.59 (dd, J=8.8, 2.0 Hz, 1H), 7.75 (s, 1H), 7.84 (J=2.0 Hz, 1H). [M+H]⁺=450, 452.

### Preparation of 3-benzyl-6-bromo-2-(1H-imidazol-1-yl) quinoline

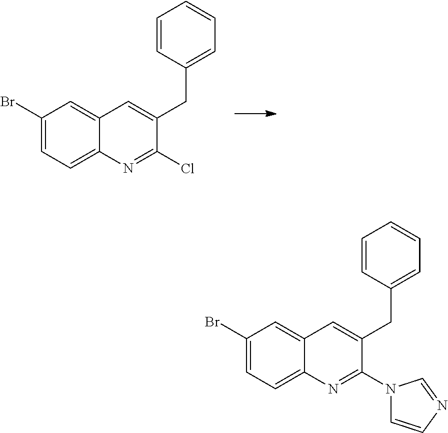

3-Benzyl-6-bromo-2-chloro quinolin (0.2 g, 0.6 mmol) and imidazole (0.2 g, 3.0 mmol) were dissolved in anhydrous pyridine (5 mL) and the mixture was heated under reflux for 12 hrs. The reaction mixture was poured into ice-water, extracted with ethyl acetate (2×10 mL), the combined organic layer was washed with water (2×10 mL) followed by brine (1×10 mL), dried over anhydrous sodium sulfate, filtered and the solvents were evaporated to obtain a sticky mass, which on purification by column chromatography (silica gel 100-200 mesh, eluted with 3-7% ethyl acetate in n-hyxane) gave pure 3-benzyl-6-bromo-2-(1H-imidazol-1-yl) quinoline (0.186 g, 85%) as a sticky mass. ¹H NMR (400 MHz, CDCl₃): δ 4.13 (s, 2H), 7.01 (d, J=6.8 Hz, 2H), 7.20 (s, 1H), 7.25-7.34 (m, 4H), 7.80 (dd, J=9.0, 2.1 Hz, 1H), 7.89 (s, 2H), 7.91-7.99 (m, 2H). [M+H]⁺=366, 368.

### Method B

### Preparation of N-(4-Bromo phenyl)-3-phenyl propionamide

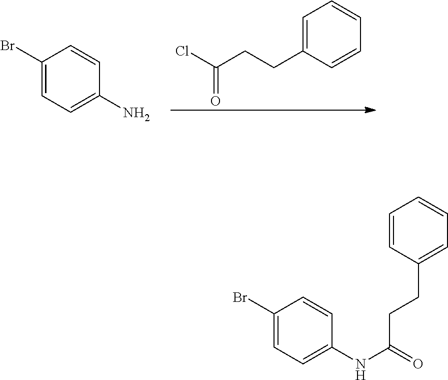

Hydrocinnamoyl chloride (19.6 g, 168.5 mmol) was added to a mixture of 4-bromoanline (10.0 g, 116.3 mmol) and triethylamine (23.5 g, 232.5 mmol) in dry dichloromethane (200 ml) at 0° C., the mixture was stirred, and allowing it to warm up to room temperature during 4 hrs. The reaction mixture was poured into ice-water mixture, the organic layer was separated, washed with 10% aqueous solution of hydrochloric acid, water and brine, dried over anhydrous sodium sulfate, filtered and concentrated in vacuo to give the crude product, which was triturated with hexane to furnish the pure product (11.0 g, 81%) as a off white solid, Mp 149-151° C. ¹H NMR (400 MHz, CDCl₃): δ 2.64 (t, J=8.0 Hz, 2H), 3.04 (t, J=8.0 Hz, 2H), 7.01 (br s, 1H, D₂O exchangeable), 6.88-7.30 (m, 3H), 7.26-7.33 (m, 4H), 7.36-7.43 (m, 2H). (M+H)⁺=302, 304.

### Preparation of 3-Benzyl-6-bromo-2-chloro-quinoline

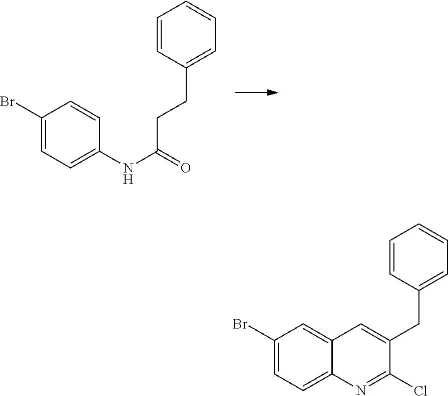

Phosphorus oxychloride (30.0 g, 196.9 mmol) was added dropwise to N,N-Dimethylformamide (14.34 g, 196.18 mmol) at 5° C., the mixture was allowed to warm up to room temperature and stirred for 20 min. The above reagent was added to a suspension of N-(4-Bromo phenyl)-3-phenyl propionamide (3.0 g, 9.86 mmol) and cetyltrimethylammonium bromide (CTAB, 0.04 g, 0.10 mmol) in acetonitrile at 5° C. The reaction mixture was heated at 80° C. for 8 h, cooled to room temperature, poured into 100 ml of 3% hypo solution at 0° C., extracted with dichloromethane, the organic layer was washed with water until the water extracts became neutral to pH paper followed by brine, dried over anhydrous sodium sulfate, filtered and concentrated under vacuum. The crude product was purified by column chromatography on silica gel (100-200) eluted with hexane-ethyl acetate (97:3) to afford the title compound (2.0 g, 64% yield) as a white crystalline solid, Mp 102° C.-104° C. ¹H NMR (400 MHz, CDCl₃): δ 4.22 (s, 2H), 7.20-7.24 (m, 2H), 7.26-7.31 (m, 1H), 7.32-7.38 (m, 2H), 7.65 (s, 1H), 7.72 (dd, J=12.0, 4.0 Hz, 1H), 7.84-7.88 (m, 2H). [M+H]⁺=332, 335.

### Preparation of 3-Benzyl-6-bromo-2-methoxy-quinoline

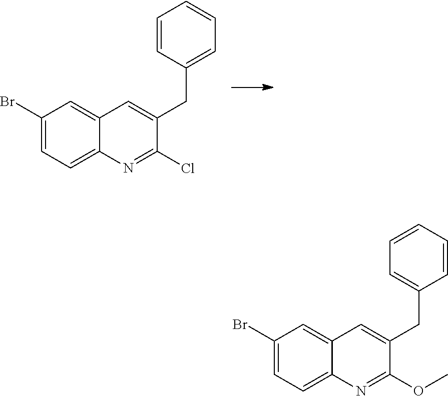

To a stirred solution of compound 3-Benzyl-6-bromo-2-chloro-quinoline (5.0 g, 15.0 mmol) in dry methanol (50 ml) was added sodium methoxide (30% w/v in methanol, 15.0 ml, 84.0 mmol) and the contents were heated under reflux for 8 h. The volatiles were removed under reduced pressure, poured into ice-water mixture; the solid separated out was filtered, washed with water and dried to furnish the compound (4.4 g, 89%) as an off-white solid, Mp 83-85° C. ¹H NMR (400 MHz, CDCl₃): δ 4.02 (s, 2H), 4.07 (s, 3H), 7.20-7.26 (m, 3H), 7.29-7.34 (m, 2H), 7.47 (s, 1H), 7.60 (dd, J=8.0, 4.0 Hz, 1H), 7.60 (dd, J=8.8, 2.2 Hz, 1H), 7.73 (d, J=2.0 Hz, 1H). (M+H)⁺=328, 330.

### Preparation of (±)-6-Bromo-3-(bromophenyl methyl)-2-methoxy-quinoline

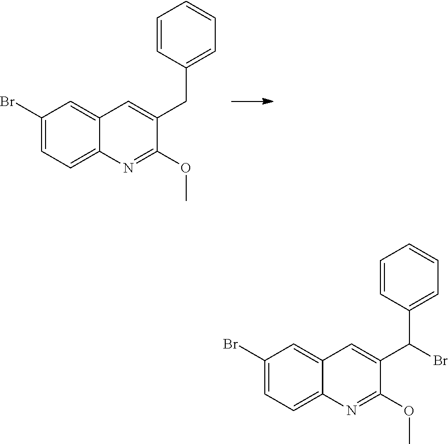

A mixture of compound 3-Benzyl-6-bromo-2-methoxy-quinoline (5.0 g, 15.20 mmol), N-Bromosuccinimide (2.7 g, 15.20 mmol) and dibenzoyl peroxide (0.18 g, 0.76 mmol) in carbon tetrachloride was heated to reflux for 2 hrs. The reaction mixture was cooled to room temperature, the solid separated out was filtered, the filtrate was concentrated under vacuum, the crude product was triturated with hexane and dried to give the compound (±)-6-Bromo-3-(bromophenyl methyl)-2-methoxy-quinoline (5.0 g, 80.6%) as an off white solid, Mp 85° C.-86° C. ¹H NMR (400 MHz, CDCl₃): δ 4.06 (s, 3H), 6.56 (s, 1H), 7.26-7.38 (m, 3H), 7.44-7.48 (m, 2H), 7.64-7.69 (m, 2H), 7.87 (d, J=4.0 Hz, 1H), 8.09 (s, 1H).

### Preparation of (±)-2-[(6-Bromo-2-methoxyquinolin-3-yl)-phenyl-methyl]-malonic acid dimethyl ester

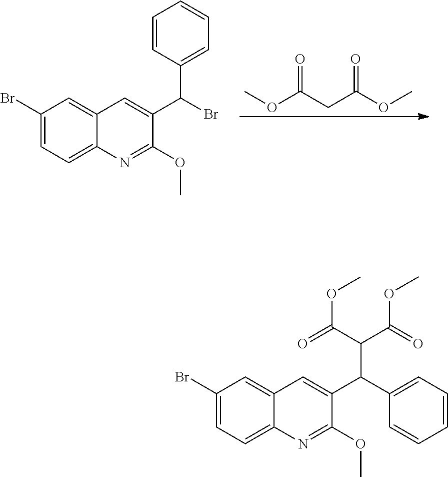

Sodium hydride (0.014 g, 0.58 mmol) was added in portions to a stirred solution of dimethyl malonate (0.08 g, 0.67 mmol) in anhydrous tetrahydrofuran (2 ml) at 0° C. and allowed to warm up to room temperature during 0.5 h. The solution of (±)-6-Bromo-3-(bromophenyl methyl)-2-methoxy-quinoline (0.20 g, 0.49 mmol) in tetrahydrofuran (2 ml) was added to the reaction mixture and stirred at room temperature for 4 h. The volatiles were removed under vacuum, poured into ice-water mixture, extracted with dichloromethane, the organic layer was washed with water, brine, dried over anhydrous sodium sulfate, filtered and concentrated under vacuum. The crude product was triturated with n-pentane and dried to give the product (±)-2-[(6-Bromo-2-methoxyquinolin-3-yl)-phenyl-methyl]-malonic acid dimethyl ester (0.20 g, 94.3% yield) as a sticky mass. ¹H NMR (400 MHz, CDCl₃): δ 3.54 (s, 3H), 3.56 (s, 3H), 4.02 (s, 3H), 4.53 (d, J=12.0 Hz, 1H), 5.12 (d, J=12.0 Hz, 1H), 7.13-7.19 (m, 1H), 7.20-7.25 (m, 2H), 7.28-7.32 (m, 2H), 7.59-7.65 (m, 2H), 7.83-7.86 (m, 2H). (M+H)⁺=458, 460.

### Preparation of 2-[(6-Bromo-2-methoxyquinolin-3-yl)-phenyl-methyl]-malonic acid monomethyl ester

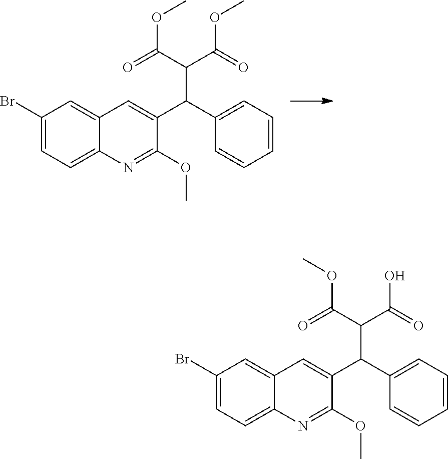

(±)-2-[(6-Bromo-2-methoxyquinolin-3-yl)-phenyl-methyl]-malonic acid dimethyl ester (3.0 g, 6.55 mmol) was added to a stirred solution of potassium hydroxide (0.36 g, 6.60 mmol) in water (5 ml) and methanol (20 ml) and heated to reflux for 12 h. The volatiles were removed under reduced pressure, poured into ice-water, extracted with diethyl ether, the aqueous layer was separated, acidified with 15% hydrochloric acid solution, extracted with chloroform, the organic layer was washed with brine, dried over anhydrous sodium sulfate, filtered and concentrated under vacuum to obtain the pure product 2-[(6-Bromo-2-methoxyquinolin-3-yl)-phenyl-methyl]-malonic acid monomethyl ester (1.40 g, 48%) as a semi solid. ¹H NMR (400 MHz, DMSO-D₆): δ 3.53 (s, 3H), 3.55 (s, 2H), 3.97 (s, 3H), 4.00 (s, 2H), 4.50-4.57 (m, 2H), 5.05-5.08 (d, 2H), 7.12-7.20 (m, 5H), 7.25-7.31 (m, 3H), 7.60-7.62 (m, 3H), 7.81-7.84 (m, 3H), 13.00 (brs, 2H), (Diastereomeric mixture in 3:2 ratio by ¹H NMR spectroscopy). (M+H)⁺=444, 446.

### Preparation of N-(4-Nitro phenyl)-3 phenyl propionamide

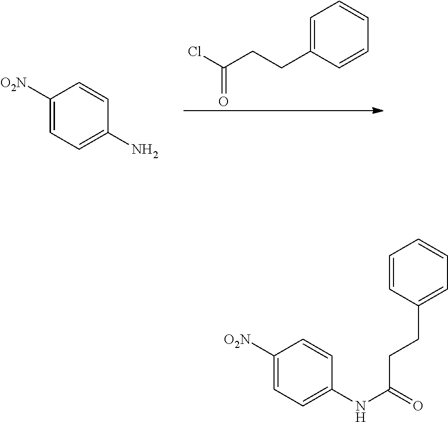

Hydrocinnamoyl chloride (21.5 ml, 144.92 mmol) was added to a mixture of 4-nitroanline (21.0 g, 144.92 mmol) and triethylamine (30.0 g, 217.40 mmol) in dry dichloromethane (400 ml) at 0° C., the mixture was stirred allowing it to warm up to room temperature during 4 h. The reaction was poured into ice-water mixture, the organic layer was separated, washed with 10% aqueous solution of hydrochloric acid, water and brine, dried over anhydrous sodium sulfate, filtered and concentrated in vacuo to give the crude product, which was triturated with hexane to furnish the pure product N-(4-Nitro phenyl)-3-phenyl propionamide (33.0 g, 84% yield) as a off white solid, Mp 117-119° C. ¹H NMR (400 MHz, CDCl₃): δ 2.72 (t, J=7.2 Hz, 2H), 3.05 (t, J=7.2 Hz, 2H), 7.18-7.41 (m, 5H), 7.59 (d, J=8.8 Hz, 2H), 8.16 (d, J=9.2 Hz, 2H). (M+H)⁺=269.

### Preparation of 3-Benzyl-6-nitro-2-chloro-quinoline

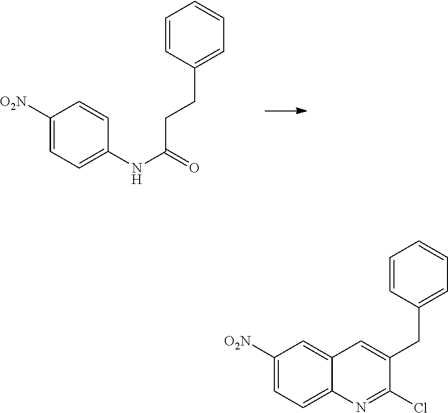

Phosphorus oxychloride (68.8 ml, 74.10 mmol) was added dropwise to N,N-Dimethylformamide (57.0 ml, 74.07 mmol) at 5° C., the mixture was allowed to warm up to room temperature and stirred for 20 minutes. The above reagent was added to a suspension of compound N-(4-Nitro phenyl)-3-phenyl propionamide (10.0 g, 37.0 mmol) and cetyltrimethylammonium bromide (CTAB, 0.04 g, 0.10 mmol) in acetonitrile at 5° C. The reaction mixture was heated at 80° C. for 8 h, cooled to room temperature, poured into 100 ml of 3% hypo solution at 0° C., extracted with dichloromethane, the organic layer was washed with water until the water extracts became neutral to pH paper followed by brine, dried over anhydrous sodium sulfate, filtered and concentrated under vacuum. The crude product was purified by column chromatography on silica gel (100-200) eluting with hexane-ethyl acetate (97:3) to afford compound 3-Benzyl-6-nitro-2-chloro-quinoline (3.40 g, 31% yield) as a white crystalline solid, Mp 159-161° C. ¹H NMR (400 MHz, CDCl₃): δ 4.26 (s, 2H), 7.24 (d, J=8 Hz, 1H), 7.29-7.40 (m, 4H), 7.89 (s, 1H), 8.11 (d, J=9.2 Hz, 1H), 8.43 (d, J=9.2, 2.4 Hz, 1H), 8.65 (d, J=2.4 Hz, 1H). (M+H)⁺=299.

### Preparation of 1-[2-(3-Benzyl-6-nitro-quinolin-2-yloxy)-5-fluoro-phenyl]-ethanone

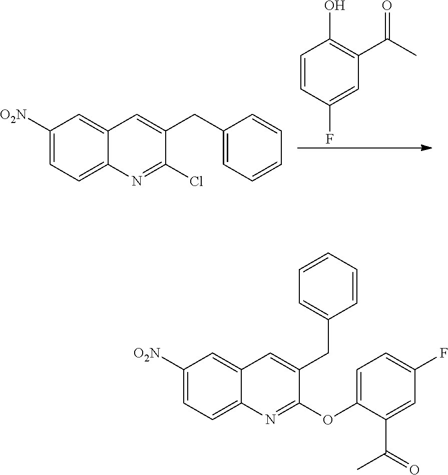

A mixture of compound 3-Benzyl-6-nitro-2-chloro-quinoline (2.0 g, 6.71 mmol), compound 1-(5-Fluoro-2-hydroxy-phenyl)-ethanone (1.13 g, 7.40 mmol) and potassium carbonate (1.11 g, 8.0 mmol) in dry dimethylsulfoxide were stirred at room temperature for 12 hrs. The mixture was poured on ice water, extracted with ethyl acetate (100 ml×3 times). The organic layer was washed with brine, dried on anhydrous sodium sulfate, filtered, concentrated under reduced pressure. The crude mixture was purified on silica gel (100-200 mesh) column chromatography, by eluting with hexane-ethylacetate (9:1) to afford compound 1-[2-(3-Benzyl-6-nitro-quinolin-2-yloxy)-5-fluoro-phenyl]-ethanone (0.7 g, 25%) as a light green colored solid, Mp 132-133° C. ¹H NMR (400 MHz, CDCl₃): δ 2.28 (s, 3H), 4.26 (s, 2H) 7.04 (dd, J=8.8, 4.4 Hz, 1H), 7.20-7.38 (m, 6H), 7.54 (dd, J=8.8, 2.8 Hz, 1H), 7.68 (d, J=9.2 Hz, 1H), 7.95 (s, 1H), 8.29 (dd, J=9.2, 2.4 Hz, 1H) 8.63 (d, J=2.4 Hz, 1H). (M+H)⁺=417.

### Preparation of 1-[2-(6-Amino-3-benzyl-quinolin-2-yloxy)-5-fluoro-phenyl]-ethanone

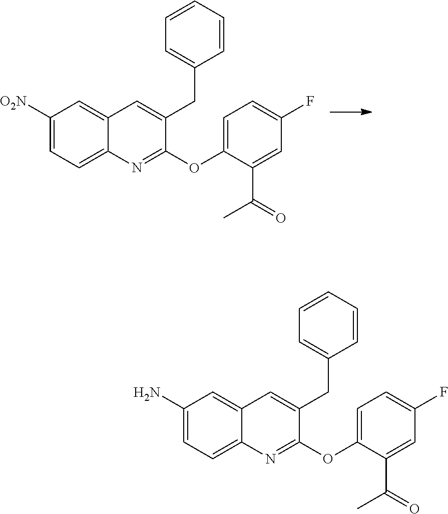

A mixture of 1-[2-(3-Benzyl-6-nitro-quinolin-2-yloxy)-5-fluoro-phenyl]-ethanone (0.30 g, 0.72 mmol) and Pd/C (0.03 g, 10% w/w) in ethyl acetate (10 ml) was stirred under hydrogen balloon pressure at room temperature for 4 h. The mixture was filtered through celite, concentrated under reduced pressure. The buff colored solid obtained was triturated with hexane, dried to get pure 1-[2-(6-Amino-3-benzyl-quinolin-2-yloxy)-5-fluoro-phenyl]-ethanone (0.240 g, 86% yield) as semi solid. ¹H NMR (400 MHz, CDCl₃): δ 2.24 (s, 3H), 4.17 (s, 2H), 6.94 (dd, J=8.8, 4.4 Hz, 1H), 7.07 (s, 1H), 7.11-7.21 (m, 3H), 7.25-7.30 (m, 6H, 2 D₂O exchangeable), 7.44 (m, 2H), 7.69 (s, 1H). (M+H)⁺=387.

### Preparation of 1-[2-(6-Azido-3-benzyl-quinolin-2-yloxy)-5-fluoro-phenyl]-ethanone

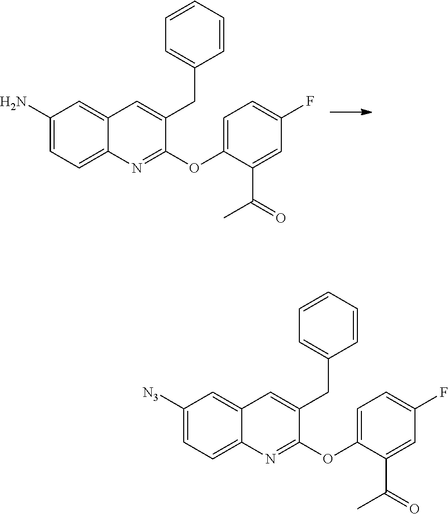

To a solution of 1-[2-(6-Amino-3-benzyl-quinolin-2-yloxy)-5-fluoro-phenyl]-ethanone (0.20 g, 0.6 mmol) in concentrated hydrochloric acid (0.3 ml), was added a solution of sodium nitrite (0.06 g, 0.84 mmol) in 0.3 ml of water, while maintaining the temperature below 5° C. Stirring for 5-10 min, the solution was added dropwise to another solution of sodium azide (0.11 g, 1.68 mmol) and sodium acetate (0.28 g, 3.36 mmol) in 2 ml of water. The mixture was stirred for 1 hour; the sticky solid was dissolved in dichloromethane (50 ml×3 times). The organic layer was dried over anhydrous sodium sulfate, filtered, concentrated and dried under reduced vacuum. The gray colored solid obtained was washed with ether to get pure 1-[2-(6-Azido-3-benzyl-quinolin-2-yloxy)-5-fluoro-phenyl]-ethanone (0.15 g, 56% yield), Mp 127-130° C. ¹H NMR (400 MHz, CDCl₃): δ 2.26 (s, 3H), 4.22 (s, 2H), 6.99 (dd, J=8.8, 4.4 Hz, 1H), 7.18-7.23 (m, 2H), 7.26-7.35 (m, 6H), 7.53 (dd, J=8.8, 2.8 Hz, 1H) 7.65 (d, J=8.8 Hz, 1H), 7.78 (s, 1H). (M+H)⁺=413.

### Preparation of 1-{2-[3-Benzyl-6-(4-phenyl-[1,2,3]triazol-1-yl)-quinolin-2-yloxy]-5-fluoro-phenyl}-ethanone

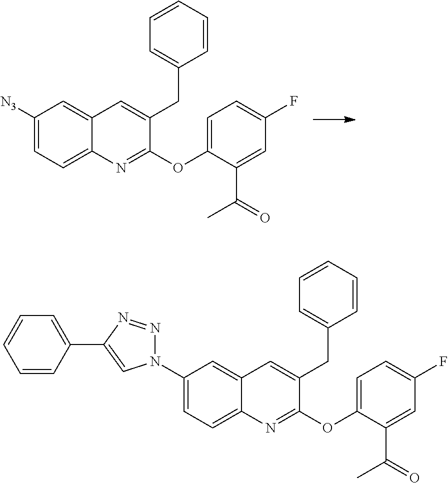

To a mixture of Phenyl acetylene (0.04 g, 0.34 mmol), Copper (I) iodide (0.063 g, 0.33 mmol) and diisopropylethylamine (0.137 g, 0.99 mmol), a solution of 1-[2-(6-Azido-3-benzyl-quinolin-2-yloxy)-5-fluoro-phenyl]-ethanone (0.14 g, 0.33 mmol) in 5 ml of acetonitrile was added dropwise at 0° C. The reaction mixture was stirred at 0° C. for 5-10 min and then 4 h at room temperature. The mixture was diluted with ethylacetate (50 ml), filtered through celite treated with 10% hydrochloric acid solution. The organic layer was dried over anhydrous sodium sulfate and concentrated under reduced pressure. The brownish solid obtained was triturated with ether to get pure 1-{2-[3-Benzyl-6-(4-phenyl-[1,2,3]triazol-1-yl)-quinolin-2-yloxy]-5-fluoro-phenyl}-ethanone (0.14 g, 82% yield), Mp 183° C. ¹H NMR (400 MHz, CDCl₃): δ 2.28 (s, 3H), 4.27 (s, 2H), 7.04 (dd, J=9.2, 4.4 Hz, 1H), 7.22 (d, J=3.2 Hz, 1H), 7.26-7.41 (m, 6H), 7.46 (t, J=7.6 Hz, 2H), 7.54 (dd, J=12.0, 3.2 Hz, 1H), 7.78 (d, J=8.8 Hz, 1H), 7.87-7.93 (m, 3H), 7.96 (dd, J=8.8, 2.4 Hz, 1H), 8.12 (d, J=2.0 Hz, 1H), 8.25 (s, 1H). (M+H)⁺=515.

### Preparation of 1-{2-[3-Benzyl-6-(4-phenyl-[1,2,3]triazol-1-yl)-quinolin-2-yloxy]-5-fluoro-phenyl}-ethanol

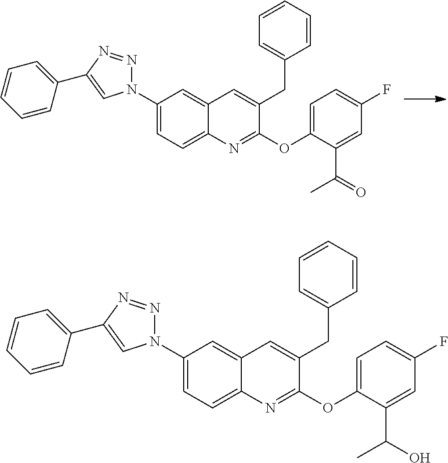

To a solution of 1-{2-[3-Benzyl-6-(4-phenyl-[1,2,3]triazol-1-yl)-quinolin-2-yloxy]-5-fluoro-phenyl}-ethanone (0.06 g, 0.116 mmol) in ethanol and tetrahydrofuran mixture (1:1, 10 ml), sodium borohydride (0.005 g, 0.12 mmol) was added at 0° C. The reaction was stirred at room temperature for 2 h. The volatiles were removed by evaporation under reduced pressure, mixture was treated with water (2 ml), extracted with ethylacetate (20 ml), dried over anhydrous sodium sulfate, filtered, concentrated under reduced pressure. The sticky solid obtained was triturated with hexane, ether to get white colored pure 1-{2-[3-Benzyl-6-(4-phenyl-[1,2,3]triazol-1-yl)-quinolin-2-yloxy]-5-fluoro-phenyl}-ethanol (0.054 g, 91% yield), Mp 103° C. ¹H NMR (400 MHz, CDCl₃): δ 1.07 (d, J=6 Hz, 3H), 4.28 (s, 2H), 4.60 (m, 1H), 5.22 (d, J=4.4 Hz, 1H, D₂O exchangeable), 7.11-7.14 (m, 2H), 7.25-7.41 (m, 7H), 7.51 (t, J=7.6 Hz, 2H), 7.78 (d, J=8.8 Hz, 1H), 7.95 (d, J=7.6 Hz, 2H), 8.18 (dd, J=8.8, 2.4 Hz, 1H), 8.33 (s, 1H), 8.49 (d, J=2.4 Hz, 1H), 9.42 (s, 1H). (M+H)⁺=517.

### Preparation of 3-Benzyl-2-[2-(1-chloro-ethyl)-4-fluoro-phenoxy]-6-(4-phenyl-[1,2,3]triazol-1-yl)-quinoline

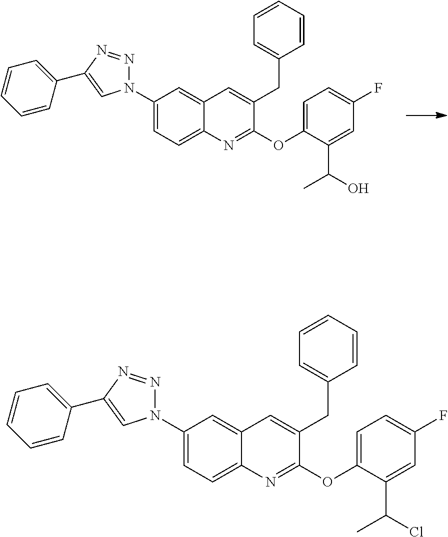

To a solution of 1-{2-[3-Benzyl-6-(4-phenyl-[1,2,3]triazol-1-yl)-quinolin-2-yloxy]-5-fluoro-phenyl}-ethanol (0.02 g, 0.03 mmol) in 1 ml of acetonitrile, thionyl chloride (0.005 g, 0.04 mmol) was added at 0° C. The mixture was stirred at room temperature for 1 h. The volatiles were removed by evaporation under reduced pressure, treated with water, extracted with ethyl acetate (25 ml). The organic layer was dried over anhydrous sodium sulfate, filtered, concentrated under reduced pressure. The crude product was triturated with hexane and dried to give the pure 3-Benzyl-2-[2-(1-chloro-ethyl)-4-fluoro-phenoxy]-6-(4-phenyl-[1,2,3]triazol-1-yl)-quinoline (0.012 g, 60% yield), Mp 151-152° C. ¹H NMR (400 MHz, CDCl₃): δ 1.60 (d, J=6.8 Hz, 3H), 4.28 (s, 2H), 4.87 (q, J=6.8 Hz, 1H), 7.01-7.08 (m, 2H), 7.27-7.41 (m, 7H), 7.46 (t, J=7.6 Hz, 2H), 7.80 (d, J=8.8 Hz, 1H), 7.91-7.97 (m, 4H), 8.14 (d, J=2.0 Hz, 1H), 8.27 (s, 1H). [M+H]⁺=535.

### Preparation of 1-[2-(2-Acetyl-4-fluoro-phenoxy)-3-benzyl-quinolin-6-yl]-3-(3-nitro-phenyl)-urea

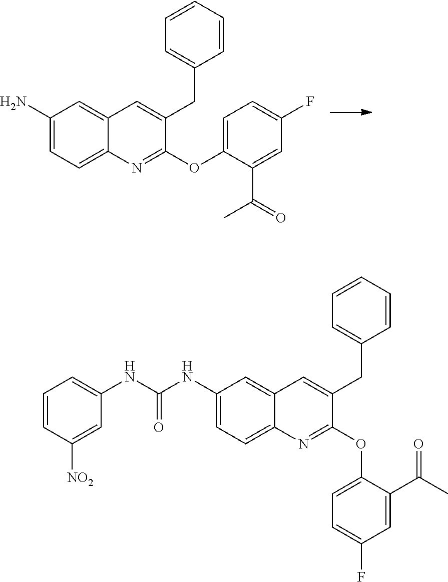

To a solution of 1-[2-(6-Amino-3-benzyl-quinolin-2-yloxy)-5-fluoro-phenyl]-ethanone (0.15 g, 0.38 mmol) and pyridine (0.015 g, 0.19 mmol) in dry dichloromethane (3 ml), 3-nitrophenyl isocyanate (0.06 g, 0.38 mmol) was added by dissolving in dry dichloromethane (1 ml) dropwise and reaction was stirred at room temperature for 12 h. The volatiles were removed under reduced pressure by evaporation. Diluted with 10% hydrochloric acid solution (15 ml), extracted with ethyl acetate. Organic layer was washed with water (10 ml×2 times), brine, dried over anhydrous sodium sulfate and concentrated under reduced pressure. The syrupy liquid obtained was triturated with hexane-pentane and dried under vacuum to get pure 1-[2-(2-Acetyl-4-fluoro-phenoxy)-3-benzyl-quinolin-6-yl]-3-(3-nitro-phenyl)-urea as semi solid. ¹H NMR (400 MHz, DMSO-d₆): δ 2.20 (s, 3H), 4.20 (s, 2H), 7.13 (dd, J=8.8, 4.6 Hz, 1H), 7.21-7.25 (m, 1H), 7.30-7.35 (m, 4H), 7.44-7.51 (m, 2H), 7.55-7.60 (m, 3H), 7.72 (d, J=8.1 Hz, 1H), 7.83 (dd, J=8.2, 1.2 Hz, 1H), 8.10 (d, J=2.0 Hz, 1H), 8.18 (s, 1H), 8.61 (s, 1H), 9.08 (s, 1H), 9.31 (s, 1H). [M+H]⁺=569.

### Napthalene-1-carbonyl chloride

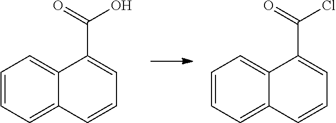

1-Napthoic acid (1.0 g, 5.81 mmol) dissolved in thionyl chloride (5 ml) and refluxed for 2 hours. Thionyl chloride was removed under reduced pressure, co-evaporated with benzene (2×5 mL) to obtain napthalene-1-carbonyl chloride (1.01 g, 98%) as a liquid. Since this acid chloride was not very stable, it was used in the next step without further purification and characterization.

### Napthalen-1-yl-phenyl-methanone

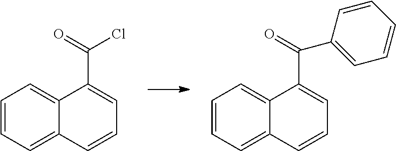

Napthalene-1-carbonyl chloride (1.03 g, 5.77 mmol) was dissolved in benzene (20 mL) and the solution was cooled to 0° C. (ice bath). Anhydrous aluminum chloride (2.30 g, 17.30 mmol) was added to this solution, the cooling bath was removed, and the reaction was stirred at rt for 2 hrs. Reaction mixture was poured into a cooled 10% aqueous solution of hydrochloric acid, extracted with ethyl acetate (2×16 mL), the combined organic layer was washed with water (2×16 mL), brine (1×16 mL), dried over anhydrous sodium sulfate, filtered and concentrated to obtain a sticky mass. Purification by column chromatography (silica gel 100-200 mesh, eluent 5% ethyl acetate in n-hexane) to obtain pure eluted the pure napthalen-1-yl-phenyl-methanone (1.10 g, 83%) as a colorless liquid. ¹H NMR (400 MHz, CDCl₃): δ 7.41-7.62 (m, 7H), 7.87 (d, J=7.7 Hz, 2H), 7.92 (d, J=7.5 Hz, 1H), 8.00 (d, J=8.1 Hz, 1H), 8.09 (d, J=8.2 Hz, 1H). [M+H]⁺=233.

### Napthalen-1-yl-phenyl-methanol

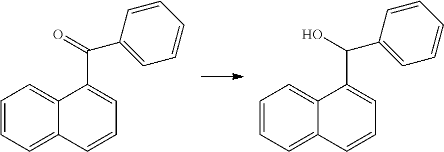

Napthalen-1-yl-phenyl-methanone (0.05 g, 0.21 mmol) was taken in ethanol (1 mL) and the mixture was cooled to 0° C. (ice bath). Sodium borohydride (0.01 g, 0.29 mmol) was added to this solution, the cooling bath was removed and the reaction was stirred at rt for 2 h. After complete disappearance of the starting material on TLC, the reaction was quenched by addition of ice pieces; the volatiles were removed under reduced pressure and extracted with ethyl acetate (2×10 ml). The combined organic layer was washed with water (2×10 ml) followed by brine (1×10 ml), dried over anhydrous sodium sulfate, filtered and concentrated to obtain pure napthalen-1-yl-phenyl-methanol (0.04 g, 79%) as a colorless liquid. ¹H NMR (400 MHz, CDCl₃): δ 2.51 (br s, 1H, D₂O exchangeable), 6.52 (s, 1H), 7.25-7.29 (m, 1H), 7.29-7.35 (m, 2H), 7.39-7.52 (m, 5H), 7.63 (d, J=7.1 Hz, 1H), 7.82 (d, J=8.2 Hz, 1H), 7.85-7.89 (m, 1H), 8.03 (d, J=7.8 Hz, 1H).

### 2-(Napthalen-1-yl-phenyl-methoxymethyl)-oxirane

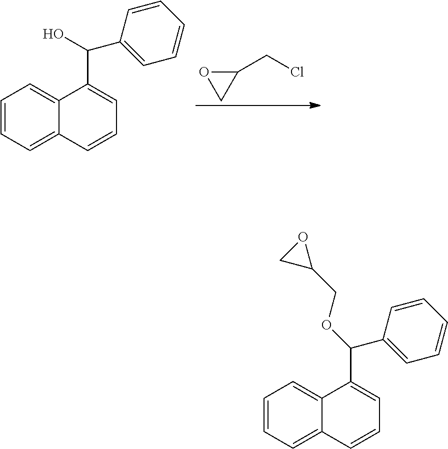

Napthalen-1-yl-phenylmethanol (0.05 g, 0.21 mmol) was dissolved in N,N-Dimethyl formamide (0.5 mL), the solution was cooled to 0° C. (ice bath), sodium hydride (0.006 g, 0.25 mmol) was added portion wise, the cooling bath was removed and the reaction was stirred at rt for half an hour, epi-chlorohydrin (0.038 g, 0.42 mmol) was added and stirring was continued for further 8 h at rt. Volatiles were removed under reduced pressure, the remaining solution was poured into ice-water mixture and extracted with ethyl acetate (2×10 ml). The combined organic layer was washed with water (2×10 ml) followed by brine (1×10 ml), dried over anhydrous sodium sulfate, filtered and concentrated to obtain a sticky mass. Purification by column chromatography (Silica gel 100-200 mesh, eluent 6% ethyl acetate in n-hexane) gave pure 2-(napthalen-1-yl-phenyl-methoxymethyl)-oxirane (0.032 g, 52%) as a colorless liquid. ¹H NMR (400 MHz, CDCl₃): δ 2.53-2.56 & 2.62-2.65 (2 m, 1H), 2.74-2.81 (m, 1H), 3.20-3.26 (m, 1H), 3.47-3.58 (m, 1H), 3.78-3.83 (m, 1H), 6.13 (s, 1H), 7.20-7.25 (m, 1H), 7.27-7.33 (m, 2H), 7.38-7.50 (m, 5H), 7.61 (d, J=7.1 Hz, 1H), 7.80 (d, J=8.2 Hz, 1H), 7.83-7.87 (m, 1H), 8.04-8.09 (m, 1H) total 18H in a diastereomeric ratio 1:1.

### Example 1

### Preparation of methyl 3-(6-bromo-2-methoxyquinolin-3-yl)-2-(morpholine-4-carbonyl)-3-phenylpropanoate

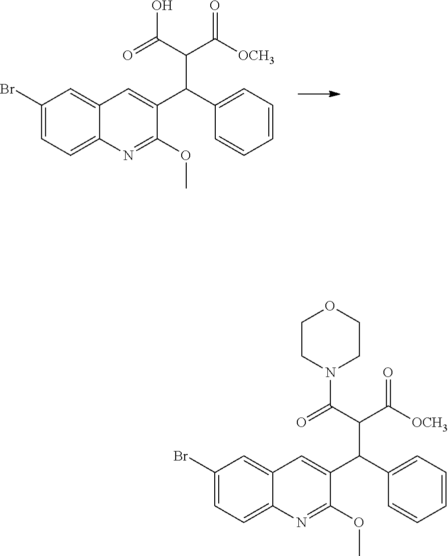

To a stirred solution of 2-[(6-Bromo-2-methoxy-quinolin-3-yl)-phenyl-methyl]-malonic acid monomethyl ester (0.60 g, 1.35 mmol), in tetrahydrofuran (10 ml) was added N-hydroxybenzotriazole (0.20 g, 1.48 mmol), morpholine (0.13 g, 1.48 mmol), 1-Ethyl-3-(3-dimethyllaminopropyl)carbodiimide hydrochloride (0.30 g, 1.62 mmol) and diisopropyl amine (0.16 g, 1.62 mmol) at 0° C. and stirred at rt for 16 h. The volatiles were removed under reduced pressure, poured into ice-water, extracted with chloroform, the organic layer was washed with brine, dried over anhydrous sodium sulfate, filtered and concentrated under vacuum. The crude product was purified by column chromatography on silica gel (230-400) eluting with hexane-ethyl acetate (7:3) to afford methyl 3-(6-bromo-2-methoxyquinolin-3-yl)-2-(morpholine-4-carbonyl)-3-phenylpropanoate (upper Spot) (0.054 gm, 27% yield), white solid, Mp 206-208° C. ¹H NMR (400 MHz, CDCl₃): δ 3.31-3.33 (m, 1H), 3.34-3.44 (m, 1H), 3.49-3.60 (m, 5H), 3.61-3.63 (m, 2H), 3.70-3.78 (m, 1H), 3.78-3.83 (m, 1H), 3.95-4.00 (m, 3H), 4.88 (d, J=11.8 Hz, 1H), 5.23 (d, J=11.8 Hz, 1H), 7.13-7.18 (m, 1H), 7.20-7.25 (m, 2H), 7.30-7.34 (m, 2H), 7.60-7.66 (m, 2H), 7.78 (s, 1H), 7.81 (s, 1H). [M+H]⁺=513, 515.

3-(6-Bromo-2-methoxy-quinolin-3-yl)-2-(morpholine-4-carbonyl)-3-phenyl-propionic acid methyl ester: Lower Spot (0.047 gm, 25%), Off-white solid, Mp 187.5-189.5° C., δ 2.99-3.02 (m, 1H), 3.23-3.32 (m, 2H), 3.38-3.42 (m, 1H), 3.48-3.52 (m, 4H), 3.55 (s, 3H), 3.99 (s, 3H), 4.72 (d, J=12.0 Hz, 1H), 5.24 (d, J=12.0 Hz, 1H), 7.14-7.28 (m, 2H), 7.21 (s, 1H), 7.22-7.28 (m, 2H), 7.60-7.65 (m, 2H), 7.82-7.87 (m, 2H). [M+H]⁺=513, 515.

### Example 2

### Preparation of (±)-6-Bromo-3-(imidazol-1-yl-phenyl-methyl)-2-methoxy-quinoline

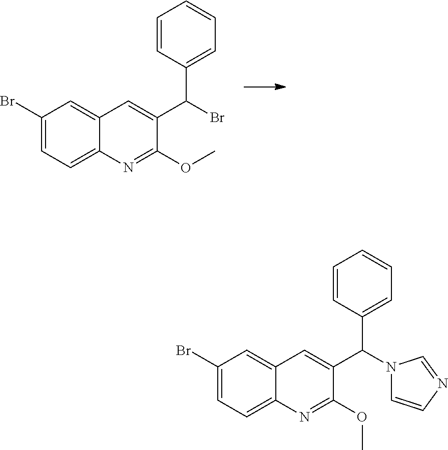

A mixture of compound (±)-6-Bromo-3-(bromophenyl methyl)-2-methoxy-quinoline (0.30 g, 0.74 mmol), imidazole (0.05 g, 0.74 mmol) and potassium carbonate (0.20 g, 1.47 mmol) in N,N-dimethylformamide (2 ml) were heated at 80° C. for 2 h. The reaction mixture was poured into ice-water mixture, extracted with ethyl acetate, the organic layer was washed with water, brine, dried over anhydrous sodium sulfate, filtered and concentrated under vacuum. The crude product was purified by column chromatography on silica gel (100-200 mesh, eluent hexane-ethyl acetate 7:3, v/v) to afford the compound (±)-6-Bromo-3-(imidazol-1-yl-phenyl-methyl)-2-methoxy-quinoline (0.07 g, 24%), off white solid, Mp 161-162° C. ¹H NMR (400 MHz, CDCl₃): δ 3.97 (s, 3H), 6.82-6.88 (m, 2H), 7.08-7.11 (m, 3H), 7.29 (s, 1H), 7.34-7.38 (m, 3H), 7.41 (s, 1H), 7.67-7.73 (m, 2H), 7.76 (d, J=1.6 Hz, 1H). [M+H]⁺=394, 396.

### Example 3

### Preparation of (±)-6-Bromo-2-methoxy-3-(phenyl-pyrazol-1-yl-methyl)-quinoline

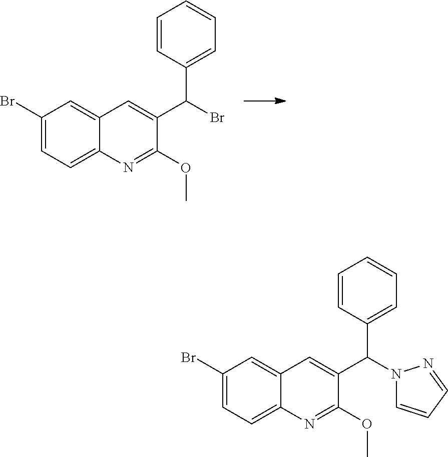

20% sodium hydroxide solution was added to a mixture of (±)-6-Bromo-3-(bromophenyl methyl)-2-methoxy-quinoline (0.30 g, 0.73 mmol), pyrazole (0.05 g, 0.73 mmol) and tetrabutyl ammonium bromide (TBAB, 0.02 g, 0.07 mmol) in toluene and heated to reflux for 12 h. The reaction mixture was cooled to room temperature, diluted with ethyl acetate and the organic layer was separated. The organic layer was washed with water, brine, dried over anhydrous sodium sulfate, filtered and concentrated under vacuum. The crude product was purified by column chromatography on silica gel (100-200 mesh) eluting with hexane-ethyl acetate (9:1) to afford the compound (±)-6-Bromo-2-methoxy-3-(phenyl-pyrazol-1-yl-methyl)-quinoline (0.08 g, 27%) as a white solid, Mp 142-144° C. ¹H NMR (400 MHz, CDCl₃): δ 3.96 (s, 3H), 6.29 (t, J=2.1 Hz, 1H), 7.03 (s, 1H), 7.11-7.15 (m, 2H), 7.28-7.30 (m, 2H), 7.33-7.37 (m, 3H), 7.61 (d, J=1.7 Hz, 1H), 7.65 (dd, J=8.8, 2.1 Hz, 1H), 7.69 (d, J=8.8 Hz, 1H), 7.75 (d, J=2.0 Hz, I H). [M+H]⁺=394, 396.

### Example 4

### Preparation of (±)-6-{[(6-Bromo-2-methoxy-quinolin-3-yl)-phenyl-methyl]-amino}-chromen-2-one

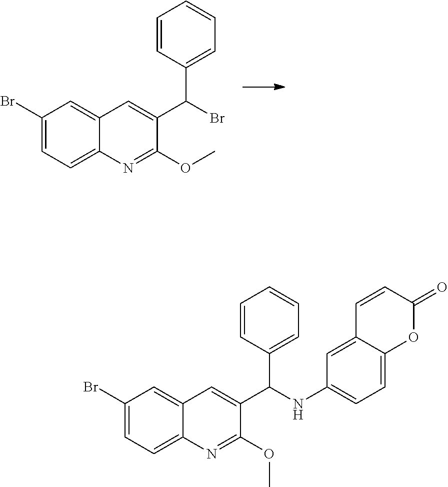

A mixture of (±)-6-Bromo-3-(bromophenyl methyl)-2-methoxy-quinoline (0.20 g, 0.49 mmol), 6-aminocoumarin hydrochloride (0.09 g, 0.5 mmol), 1,8-diazabicyclo-[5.4.0]undec-7-ene (0.07 ml, 0.5 mmol), tetrabutylammonium bromide (0.03 g, 0.09 mmol) and potassium carbonate in toluene were heated under reflux for 12 h. The reaction mixture was cooled to room temperature, poured into water, diluted with ethyl acetate and the organic layer was separated. The organic layer was washed with water followed by brine, dried over anhydrous sodium sulfate, filtered and concentrated under vacuum. The crude product was purified by column chromatography on silica gel (100-200 mesh) eluting with hexane-ethyl acetate (9:1, v/v) to afford the compound (═)-6-{[(6-Bromo-2-methoxy-quinolin-3-yl)-phenyl-methyl]-amino}-chromen-2-one (0.03 g, 12%) as a pale yellow solid, Mp 88-89° C. ¹H NMR (400 MHz, CDCl₃): δ 4.02 (s, 3H), 4.33 (d, J=3.6 Hz, 1H), 5.77 (d, J=3.7 Hz, 1H), 6.32 (d, J=9.5 Hz, 1H), 6.44 (d, J=2.8 Hz, 1H), 6.80 (dd, J=9.0, 2.8 Hz, 1H), 7.13 (d, J=8.8 Hz, 1H), 719-7.34 (m, 5H), 7.50 (d, J=5.6 Hz, 1H), 7.65 (dd, J=9.0, 2.4 Hz, 1H), 7.70 (d, J=8.8 Hz, 1H), 7.82 (d, J=1.6 Hz, 1H), 7.98 (s, 1H). [M+H]⁺=486, 488.

### Example 5

### Preparation of 3-Benzyl-2-[4-fluoro-2-(1-imidazol-1-yl-ethyl)-phenoxy]-6-(4-phenyl-[1,2,3]triazol-1-yl)-quinoline

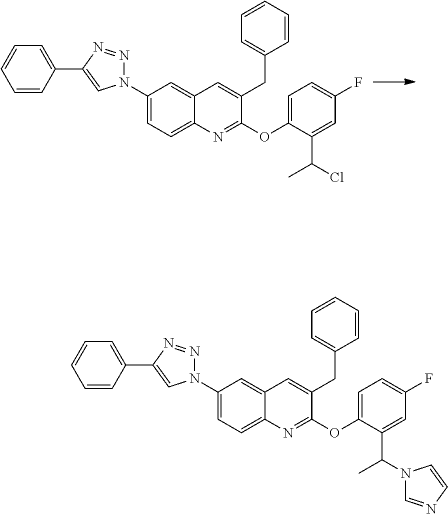

A mixture of 3-Benzyl-2-[2-(1-chloro-ethyl)-4-fluoro-phenoxy]-6-(4-phenyl-[1,2,3]triazol-1-yl)-quinoline (0.02 g, 0.03 mmol), imidazole (0.015 g, 0.22 mmol), triethylamine (0.022 g, 0.22 mmol) in acetonitrile (1 ml) was heated to reflux in a sealed tube for 12 h. The volatiles were removed under reduced pressure. The mixture was treated with water (10 ml), extracted with ethylacetate (25 ml×2 times), dried over anhydrous sodium sulfate, filtered, concentrated under reduced pressure. The crude mixture was purified by column chromatography on neutral alumina eluted with 3% chloroform-methanol to obtain 3-Benzyl-2-[4-fluoro-2-(1-imidazol-1-yl-ethyl)-phenoxy]-6-(4-phenyl-[1,2,3]triazol-1-yl)-quinoline (0.012 g, 60%) as a white solid. Mp 118-120° C. ¹H NMR (400 MHz, DMSO-d₆): δ 1.55 (d, J=6.8 Hz, 3H), 4.30 (s, 2H), 5.18 (q, J=8.8 Hz, 1H), 6.78 (s, 1H), 7.07 (s, 1H), 7.16-7.24 (m, 4H), 7.30-7.42 (m, 6H), 7.51 (t, J=7.6 Hz, 2H), 7.77 (d, J=8.8 Hz, 1H), 7.96 (d, J=8.0 Hz, 2H), 8.18 (dd, J=6.4, 2.0 Hz, 1H), 8.30 (s, 1H), 8.51 (d, J=2.0 Hz, 1H), 9.45 (s, 1H). [M+H]⁺=567.

### Example 6

### Preparation of 1-{3-Benzyl-2-[4-fluoro-2-(1-hydroxy-ethyl)-phenoxy]-quinolin-6-yl}-3-(3-nitro-phenyl)-urea

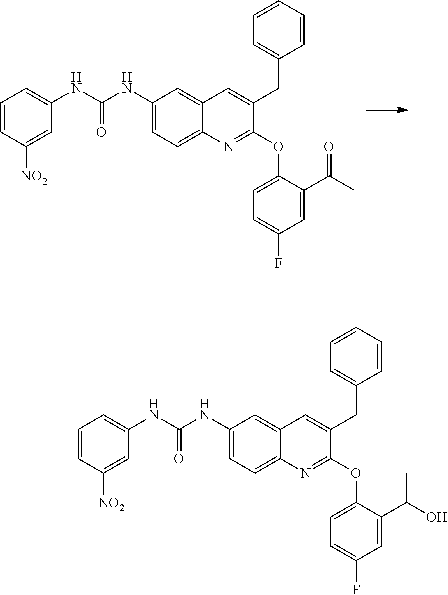

To a solution of 1-[2-(2-Acetyl-4-fluoro-phenoxy)-3-benzyl-quinolin-6-yl]-3-(3-nitro-phenyl)-urea (0.17 g, 0.30 mmol) in ethanol/tetrhydrofuran mixture (1:1, v/v, 4 ml), sodium borohydride (0.03 g, 0.77 mmol) was added at 0° C. Then the reaction was stirred at room temperature for 2 h. The volatiles were removed under reduced pressure by evaporation, treated with water (20 ml), extracted with ethylacetate (25 ml×2 times). The organic layer was dried over anhydrous sodium sulfate, filtered, concentrated under vacuo. The yellow solid after pentane wash gave pure 1-{3-Benzyl-2-[4-fluoro-2-(1-hydroxy-ethyl)-phenoxy]-quinolin-6-yl}-3-(3-nitro-phenyl)-urea (0.16 g, 94%) as white solid Mp 217-219° C. ¹H NMR (400 MHz, DMSO-d₆): δ 1.04 (d, J=6.3 Hz, 3H), 4.20 (s, 2H), 4.56-4.59 (m, 1H), 5.19 (d, J=4.3 Hz, 1H), 7.00-7.03 (m, 1H), 7.06-7.09 (m, 1H), 7.21-7.24 (m, 1H), 7.29-7.34 (m, 5H), 7.49 (d, J=9.0 Hz, 1H), 7.55-7.59 (m, 2H), 7.72 (d, J=7.8 Hz, 1H), 7.84 (d, J=1.2 Hz, 1H), 8.10 (d, J=1.9 Hz, 1H), 8.19 (s, 1H), 8.61 (d, J=1.8 Hz, 1H), 9.08 (s, 1H), 9.32 (s, 1H). [M+H]⁺=553.

### Example 7

### 1-[4-(3-Methoxy-phenyl)piperazin-1-yl]-3-(napthalen-1-yl-phenyl-methoxy)-propan-2-ol

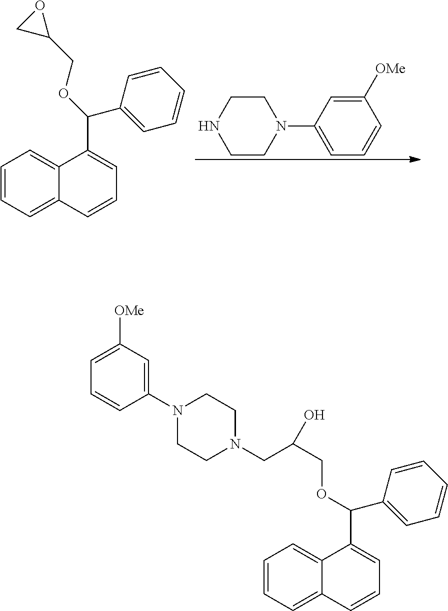

To the solution of 2-(Napthalen-1-yl-phenyl-methoxymethyl)-oxirane (0.05 g, 0.17 mmol) in 2-Propanol (5 mL) was added 1-(3-methoxy phenyl) piperazine (0.045 g, 0.17 mmol) and this mixture was refluxed for 16 hrs. The volatiles were removed under reduced pressure, the remaining thick liquid was poured into ice-water mixture and extracted with ethyl acetate (2×10 ml). The combined organic layer was washed with water (2×10 ml) followed by brine (1×10 ml), dried over anhydrous sodium sulfate, filtered and concentrated to obtain a sticky mass. Purification was carried out by washing with n-hexane (2×5 ml) followed by n-pentane (2×5 ml) to obtain pure 1-[4-(3-methoxy-phenyl)piperazin-1-yl]-3-(napthalen-1-yl-phenyl-methoxy)-propan-2-ol (0.025 g, 30%) as a light red solid. Mp 84° C. ¹H NMR (400 MHz, CDCl₃): δ 2.70-3.20 (m, 6H), 3.32-3.44 (m, 4H), 3.47-3.55 (m, 2H), 3.62-3.74 (m, 2H), 3.77 (s, 3H), 6.03 (d, J=3.5 Hz, 1H), 6.41-6.49 (m, 3H), 7.17 (t, J=8.2 Hz, 1H), 7.28-7.54 (m, 9H), 7.78-7.85 (m, 2H), 8.00 (d, J=7.6 Hz, 1H). [M+H]⁺=483.

### Section-2

### General Preparation

### Conformationally Constrained Quinoline Compounds

In particular, the compounds formula IV can be prepared by reacting an intermediate compound of formula (51) with appropriate oxime derivatives according to the Schemes 8, 9 and 10.

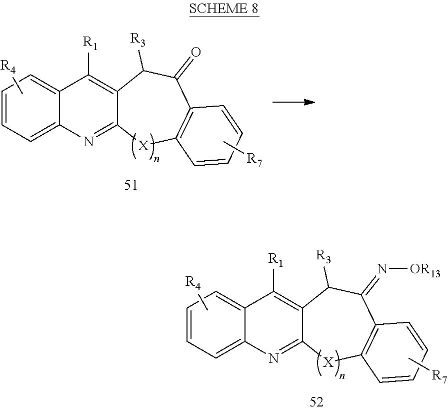

The key intermediate 51 can be prepared as per Scheme 9

Compound 51 was obtained by displacement of the chlorine in 53 by a suitable cyano substituted aryl nuchelphile under heating condition at temperature ranging from 50-150° C. which was then cyclized by under base catalyzed condition to obtain the key intermediate 51.

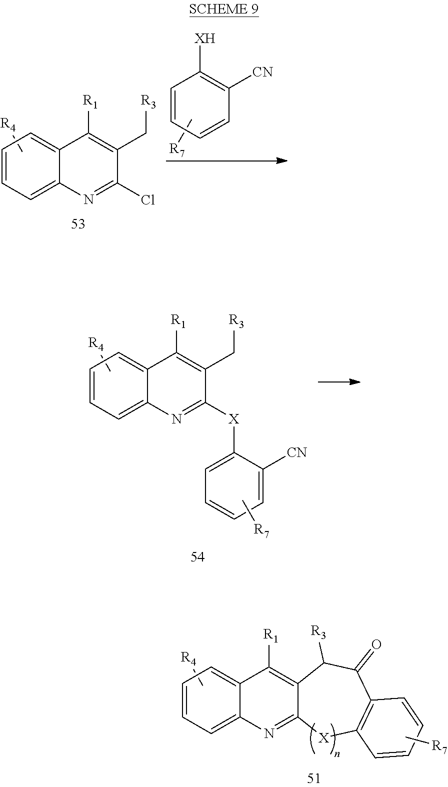

For synthesis of the intermediate 56, the initial displacement reaction was carried out using a acylated aryl nucleophile to obtain 55, which was cyclized under base catalyzed conditions.

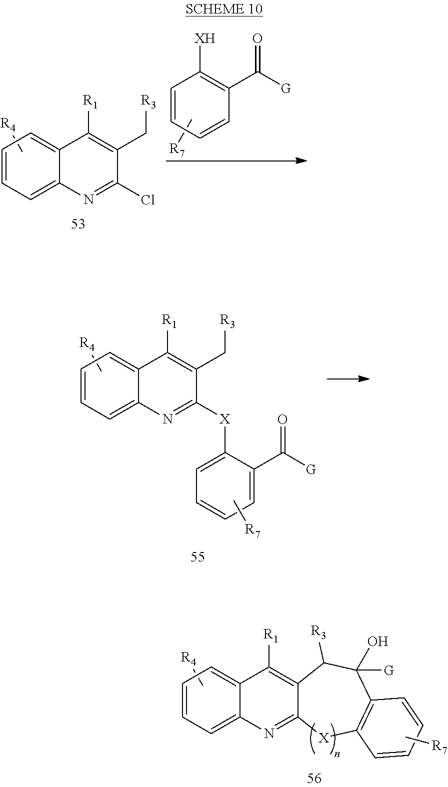

The compounds according to formula V (eg. 58) can be synthesized by reacting an intermediate 57 with an appropriate nucleophile G (G is explained in Table 1) as described in Scheme 11.

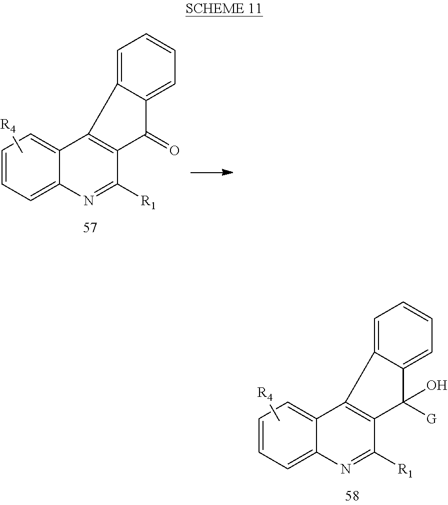

The required intermediate (57) for the synthesis of compound formula 58 can be achieved according to Scheme 12. Iso-oxazole 60 can be synthesized by reacting an appropriate nitro aromatic compound 59 with a substituted aryl acetonitrile under the influence of a suitable base at temperature ranging from 0° C. to 100° C. (Mamo, A.; Nicoletti, S.; Tat, N. C Molecules. 2002, 7, 618-627). Reduction of iso-oxazole followed by coupling with malonic acid provides synthesis for 62, which can be easily cyclized to 63 under the influence of a suitable Lewis acid. The chlorine in 63 can be substituted by any appropriate nucleophile under nucleophilic substitution condition at temperature ranging from 50-150° C.

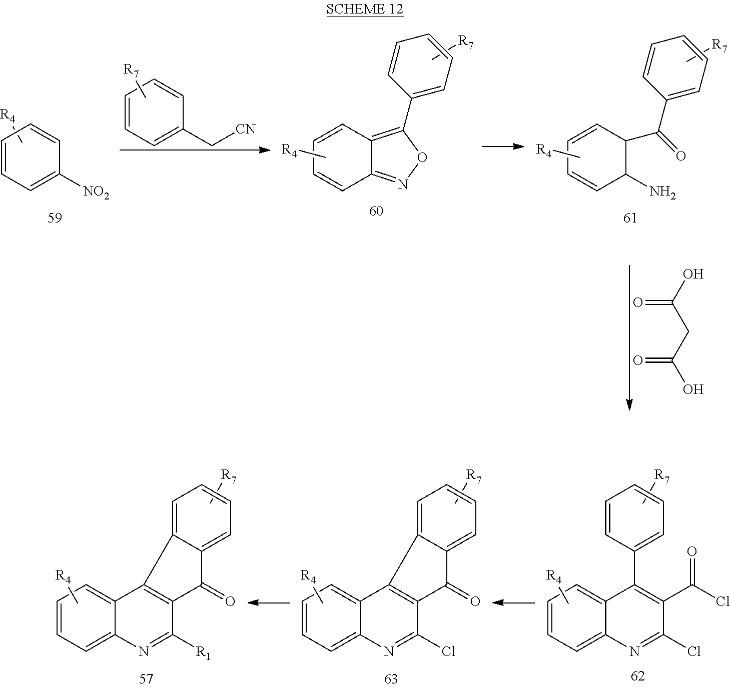

The synthesis of compounds represented by formula V (eg. 65) can be achieved by reacting intermediate (64) with an appropriate nucleophile G (G as explained in Table 1) according to Scheme 13.

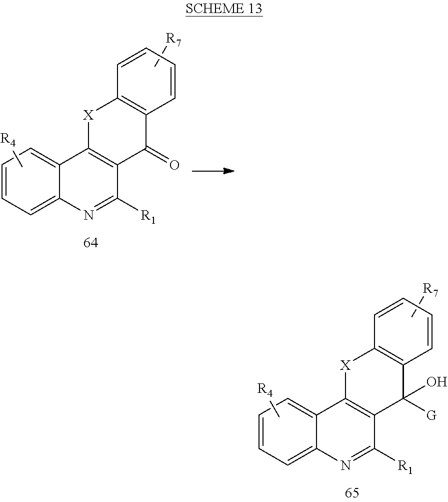

Intermediate (64) may be prepared according to the following reaction Scheme 14.

Suitably substituted aniline 39 was treated with malonic acid and phosphoric oxychloride under heating condition between temperature 50-100° C. to give the dichloroquinoline derivative 66. Substitution under controlled nucleophilic condition with a nucleophile R₁H gave the compound 67. Reaction of 67 with an appropriate nitrite gave the 68. Hydrolysis of the nitrile in 68 followed by cyclization by treatment with polyphosphoric acid gave the intermediate 64.

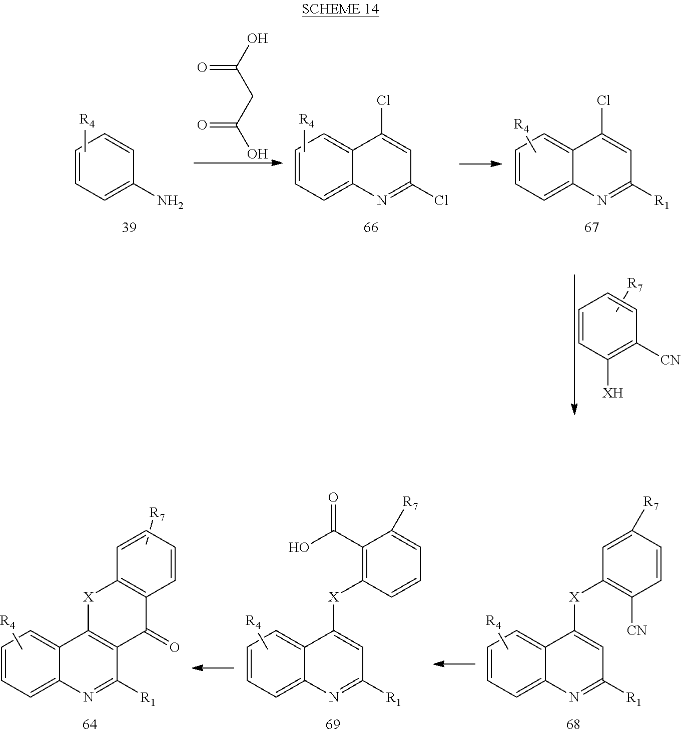

Compound 70 or 71 (Scheme 15) can be synthesized by reducing the ketone 57 or 64 using hydrazine hydrate in 1,2-ethane diol at temperature ranging from 50-200° C.

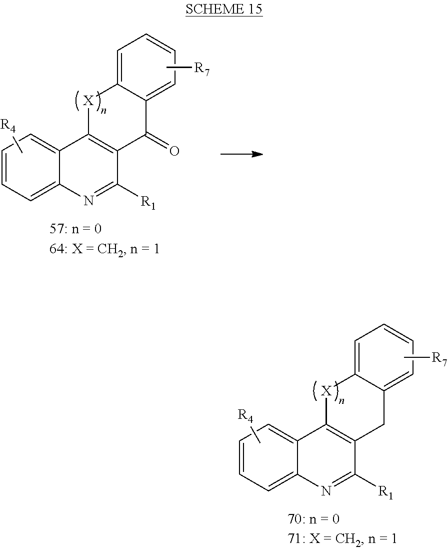

Syntheses of compound 72 or 73 (Scheme 16) can be achieved by treatment of 70 or 71 with any carbonyl compound (or compounds bearing a suitable nucleophilic center) in presence of a suitable base (n-butyl lithium and N,N-diisopropyl amine or sodium hydride) at temperature lunging from −78° C.-room temperatures.

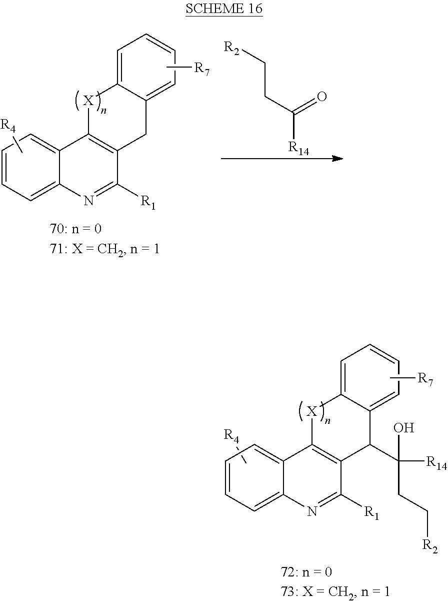

### Conformationally Constrained Naphthalene Compounds

In particular, the compounds formula VI can be prepared by opening the oxirane of formula 74 or 75 with a suitable nucleophile R₂H (R₂ is described in Table 1) as per Scheme 17.

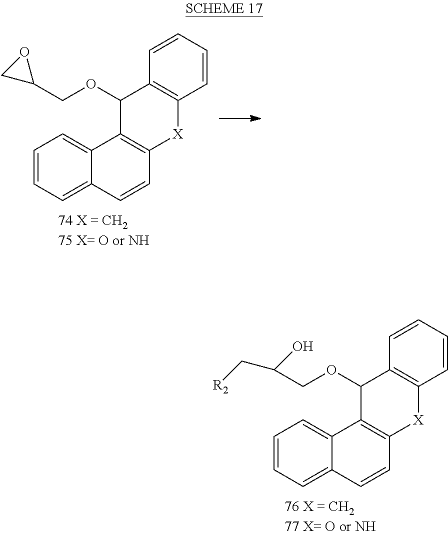

The key intermediate oxirane 74 (where X═CH₂) can be synthesized according to the Scheme 18 described below. o-Toluic acid was converted to the corresponding acid chloride by treatment with a suitable chlorinating agent such as thionyl chloride of phosphoric oxychloride and this acid chloride was subjected to Friedal-Craft acylation with naphthalene under the influence of a suitable Lewis acid to give the ketone 80. Chlorination under free redical condition with N-chlorosuccinimide and dibenzoyl peroxide gave 81. Friedal-Craft alkylation gave the phenone 82. Reduction of the ketone 82 with a hydride transfer reagent like sodium borohydride or lithium aluminum hydride gave alcohol 83, which on treatment with epi-chlorohydrin under the influence of a strong base like sodium hydride gave the intermediate oxirane 74.

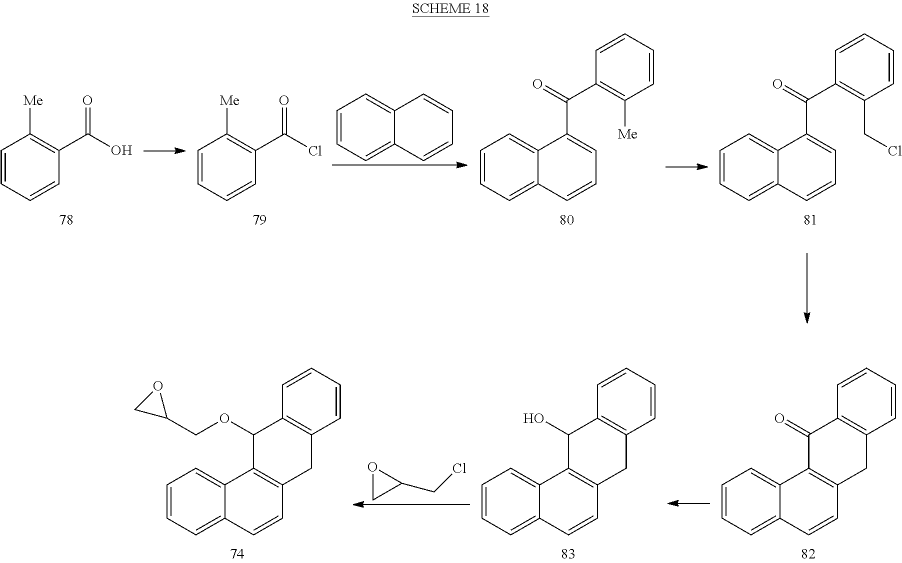

The key intermediate oxirane 75 (where X═O or N) can be synthesized according to the Scheme 19. The suitable protected carboxylic acid 84 was converted to the corresponding acid chloride by treatment with a chlorinating agent such as thionyl chloride or phosphoric oxichloride, which on treatment with 2-bromonaphthalene under Friedel-Craft acylation condition gave ketone 86. Deprotection followed by palladium catalyzed coupling of 86 gave the cyclized product 88. Reduction with a suitable hydride transfer reagent such as sodium borohydride followed by etherification with epi-chlorohydrin under the influence of a strong base such as sodium hydride gave the oxirane intermediate 75.

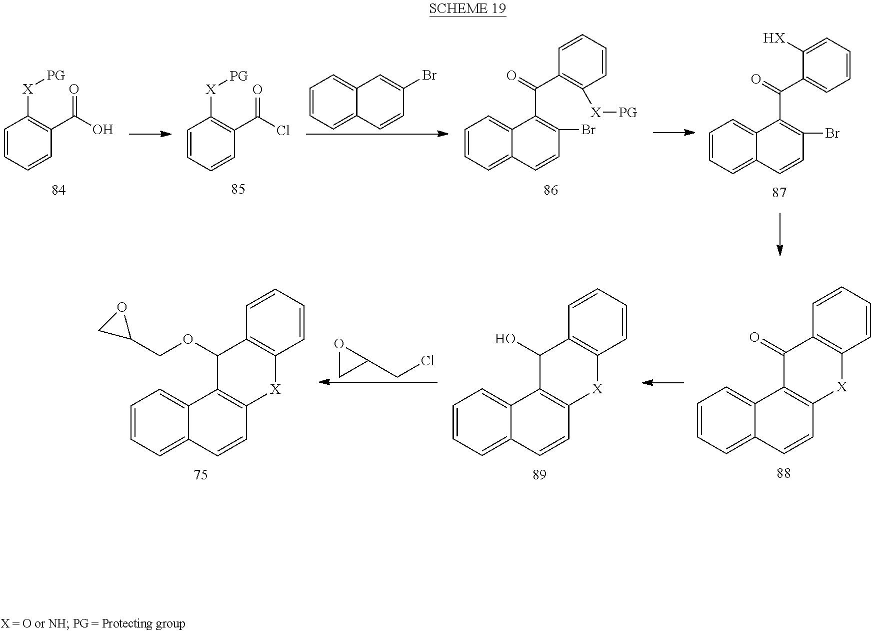

The compounds with general formula VII can be prepared by opening the oxirane of formula 90 or 91 with a suitable nucleophile R₂H (R₂ is explained in Table 1) as described in Scheme 20.

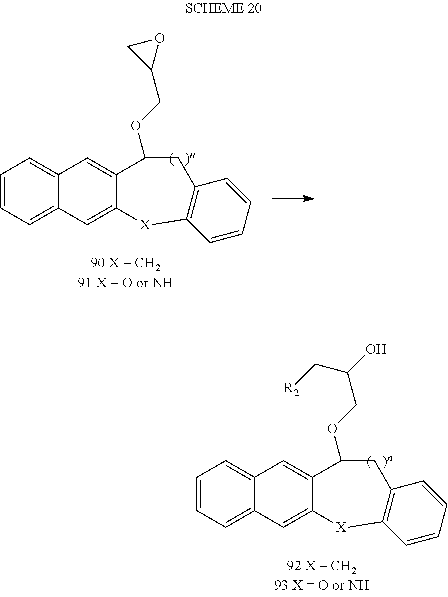

The synthesis of the key oxirane intermediate 90 (where X═CH₂) starts with the Friedal-Craft acylation at the 3-position of 2-bromomethylnaphthalene with an appropriate, freshly prepared acid chloride using a suitable Lewis acid catalyst (Scheme 21). The intramolecular Friedal-Craft cyclization of 96 gave the cyclic ketone 97, which on reduction with a suitable hydride transfer reagent such as sodium borohydride or lithium aluminum hydride gave the alcohol 98. Etharification with epi-chlorohydrin of 98 gave the key oxirane 90.

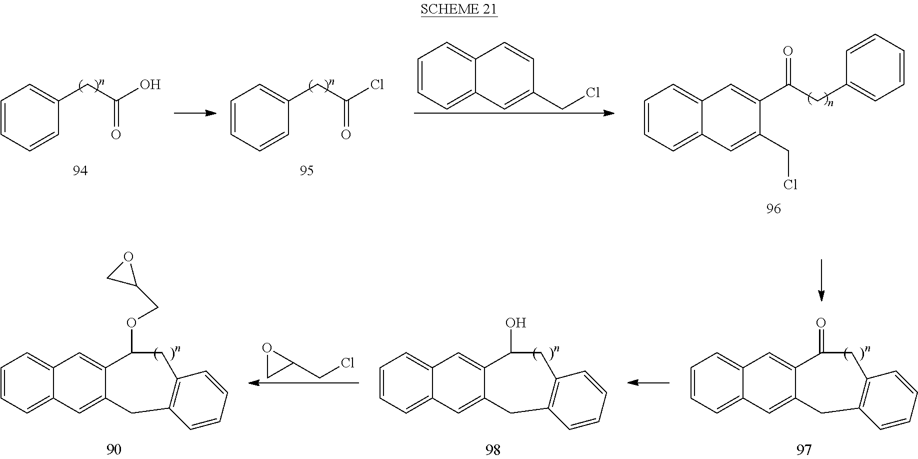

For the synthesis of the key oxiran 91 (where X═O or NH), the Scheme 22 was followed. The acid chloride of a suitable carboxylic acid 99 was treated with a suitably protected 2-naphthol (X═O) or 2-naphthylamine (X═NH) under Friedal-Craft acylation condition to obtain 101. Deprotection of 101 followed by cyclization under palladium-catalyzed condition gave the cyclic ketone 103. Reduction of this ketone with a hydride transfer reagent followed by etherification with epi-chlorohydrin gave the key oxiran 91.

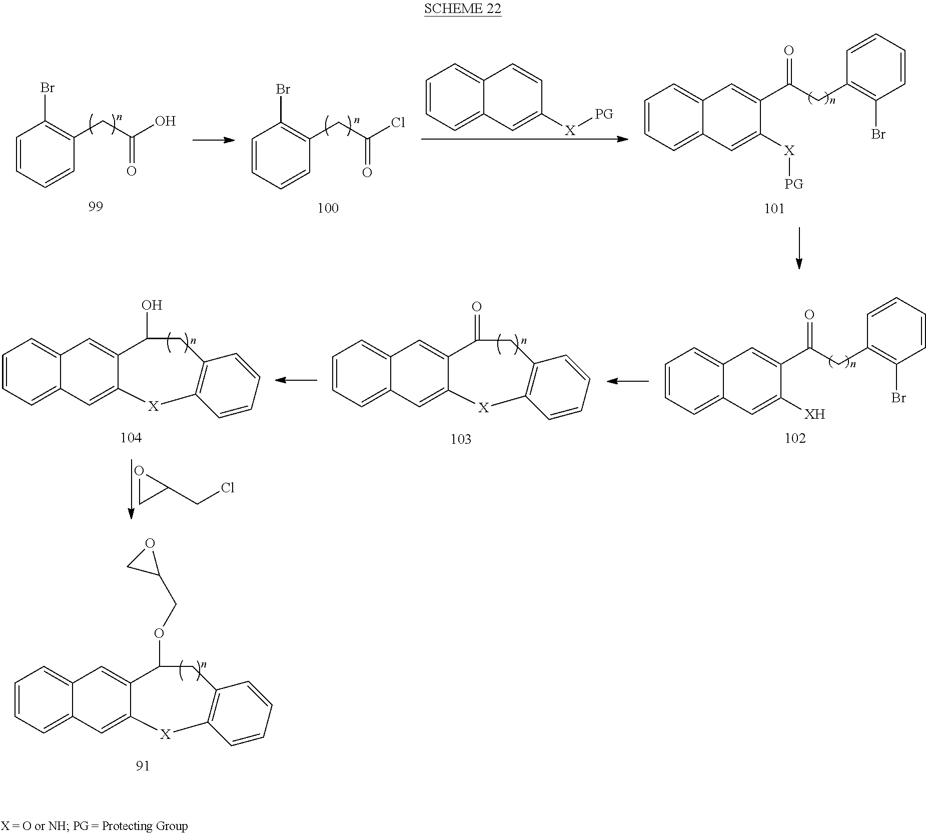

The compounds with structure VIII were synthesized by opening the oxiranes of formula 105 or 106 or 107 (as shown in Scheme 23) by a suitable nucleophile R₂H(R₂ is described in Table 1) under neutral to basic condition between rt and reflux temperature.

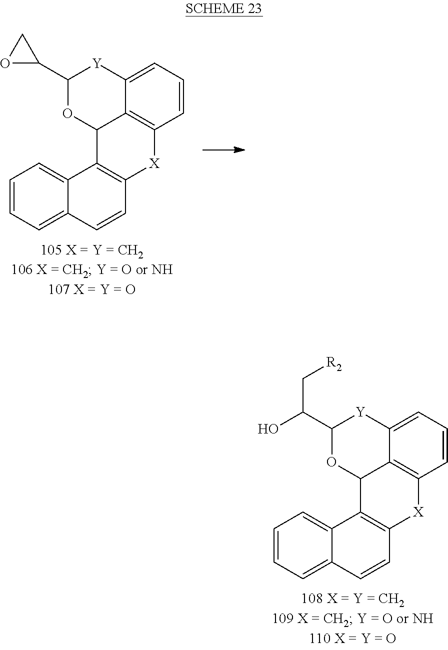

The key oxirane 105 (where X═Y═CH₂) is synthesized according to Scheme 24. The compound 83 (Scheme 18) was treated with 2-vinyl oxirane under boron trifluoride catalyzed condition to give 111, which on treatment with thionyl chloride gave the chloride 112. The indium chloride catalyzed intramolecular Friedel-Craft alkylation gave the cyclic compound 113. The oxirane was formed on the double bond by epoxidation with 3-chloro perbenzoic acid to obtain oxirane 105 as the key intermediate.

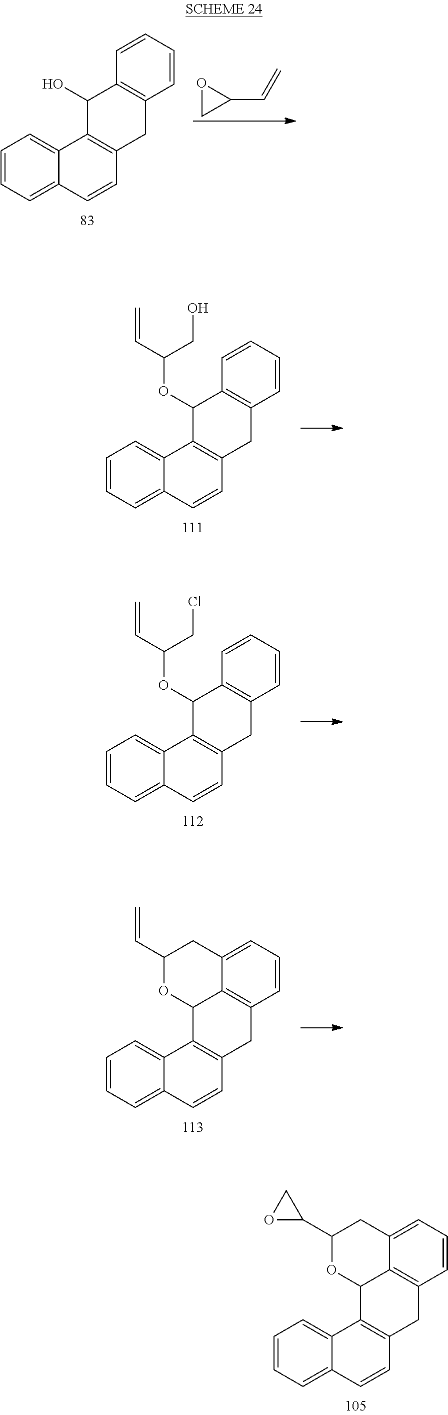

The key oxirane 106 (where X═CH₂; Y═O or N) can be synthesized according to Scheme 25. A suitably protected aromatic ester was converted to the corresponding acid chloride 115 by treatment with phosphoric oxychloride under reflux. The acid chloride then condensed to naphthalene by Friedal-Craft acylation technique to obtain 116. Chlorination of the methyl group in 116 with N-chlorosuccinimide gave the corresponding chlo compound 117, which on treatment with a Lewis acid gave the cyclized compound 118. This compound was reduced to obtain alcohol 119, which was treated with 2-vinyl oxirane under boron trifluoride catalyzed condition gave 120. On treatment with thionyl chloride, 120 gave the chloride 121. Deprotection of the protecting group followed by cyclization under base catalyzed nucleophilic substitution condition gave 123. The key oxirane 106 was obtained by epoxidation of 123 with 3-chloro perbenzoic acid.

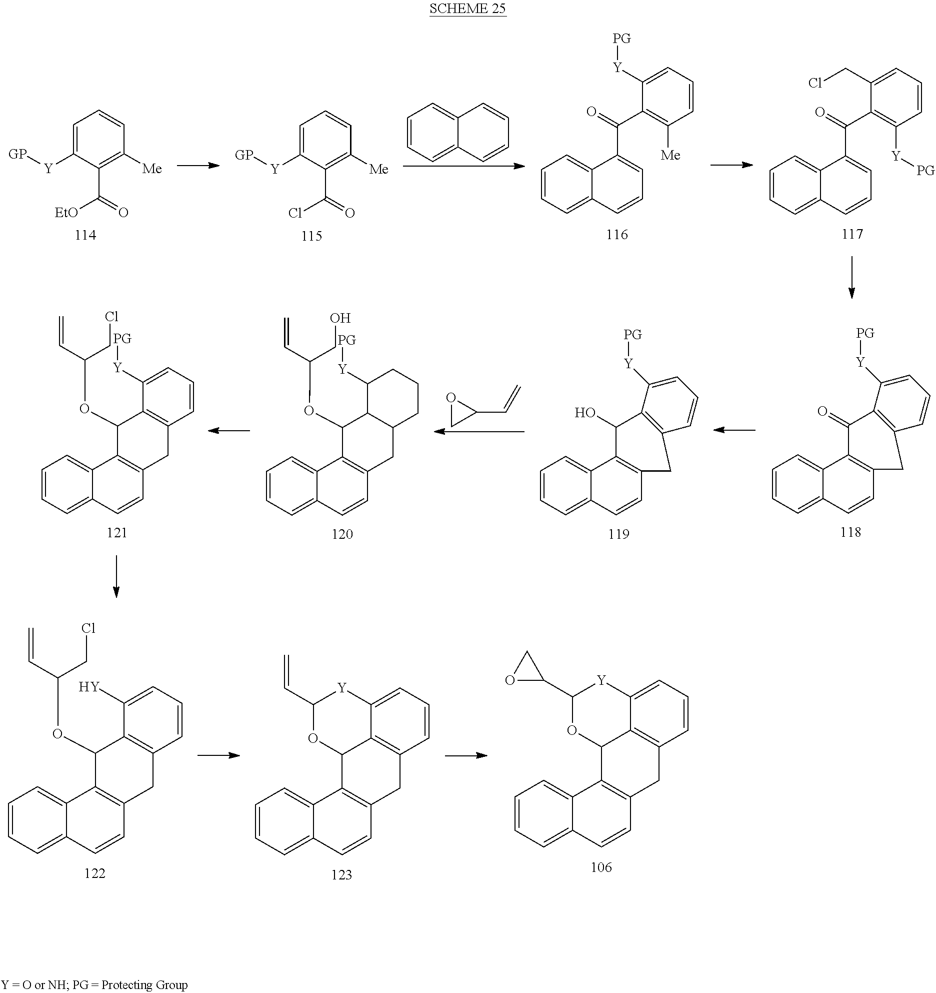

The key oxirane 107 (where X═Y═O) can be prepared according to Scheme 26. 2,6 dimethoxy benzoicacid was converted to the corresponding acid chloride 125 by treatment with thionyl chloride under reflux. The acid chloride then condensed to 2-bromonaphthalene by Friedel-Craft acylation technique to obtain 126. Removal of the methyl groups under Lewis acid catalyzed demethylation condition gave the diol 127. When subjected to the palladium catalyzed coupling condition, this diol was converted to 128. The remaining hydroxy group was protected to obtain 129. This compound was reduced with a hydride transfer reagent to obtain alcohol 130. The alcohol 130 was treated with 2-vinyl oxirane under boron trifluoride catalyzed condition gave 131, which on treatment with thionyl chloride gave the chloride 132. Deprotection of the protecting group followed by cyclization under base catalyzed nucleophilic substitution condition gave 134. The key oxirane 107 was obtained by epoxidation of 134 with 3-chloro perbenzoic acid.

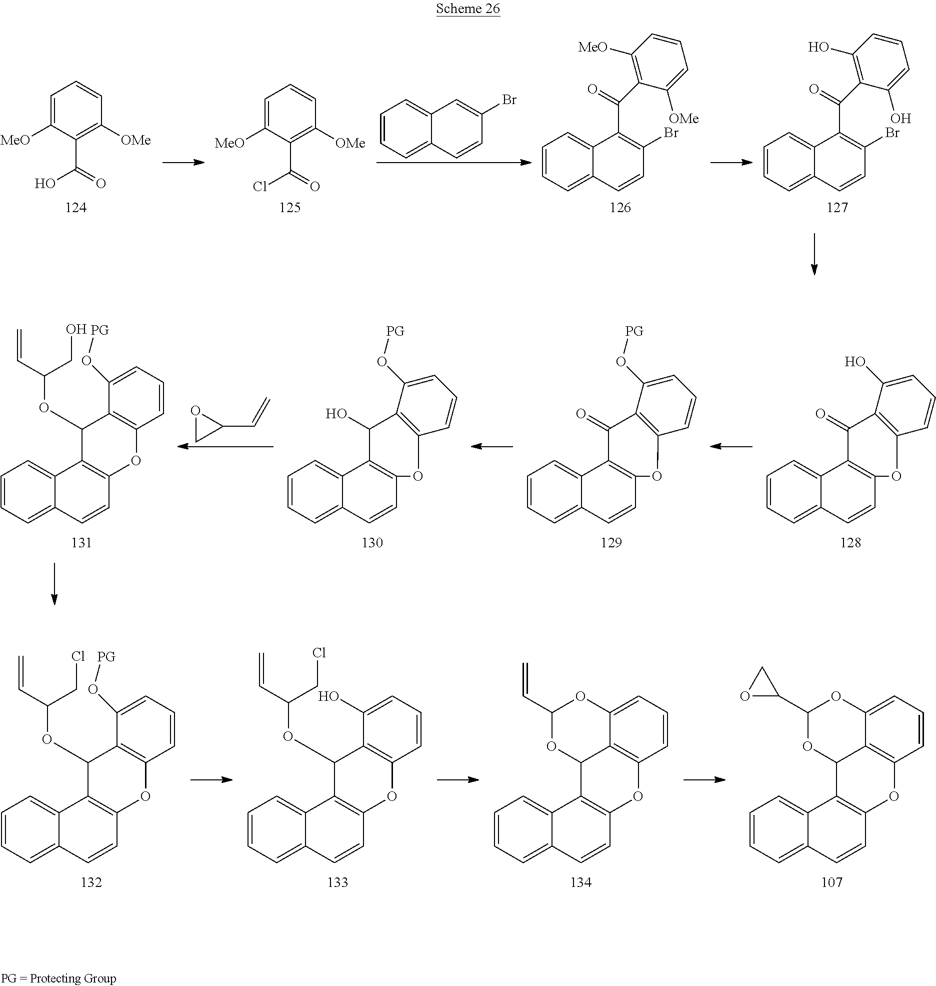

The key oxirane 139 can be prepared according to Scheme 27. A suitably protected quinilone derivative 66 was converted to ester 135 by treatment of LDA followed by ethyl chloroformate, 2-Chloro was nucleophilic substituted by different nucleophilies, and then ester was converted to acid 137 by basic hydrolysis. This acid on treatment of lewis acid gave cyclised product 138. Etherification of 138 with epi-chlorohydrin gave the key oxiran 139.

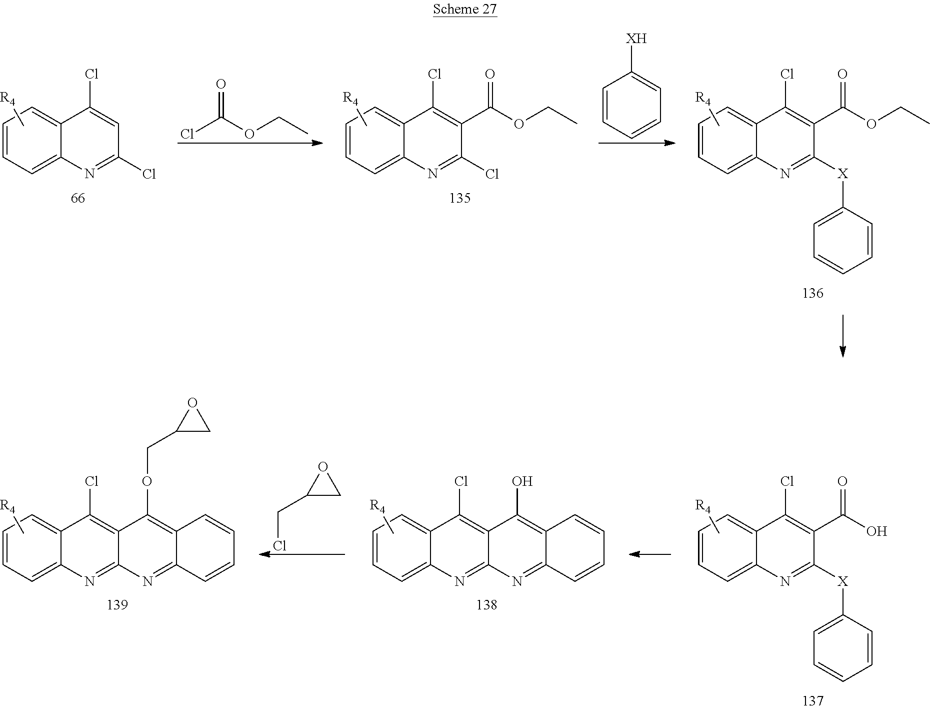

The key oxirane 145 can be prepared according to Scheme 28. A suitably protected quinilone derivative 141 was synthesized by nucleophilically substitution of 2-Chloro in 140 by different nucleophilies, and then ester was converted to acid 142 by basic hydrolysis. This acid on treatment of lewis acid gave cyclised product 143. Compound 143 was reduced by sodium borohydride treatment to get alcohol 144. Etherification of 144 with epi-chlorohydrin gave the key oxiran 145.

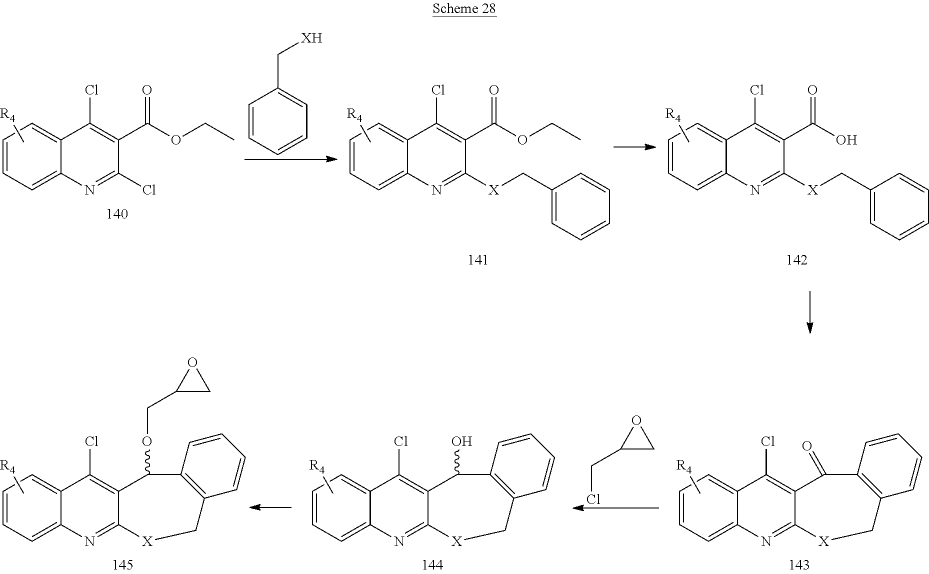

### Experimental Part Two

Preparation of Intermediates for Conformationally Constrained Compounds:

### Preparation of 5-Bromo-3-phenyl-benzo[c]isoxazole

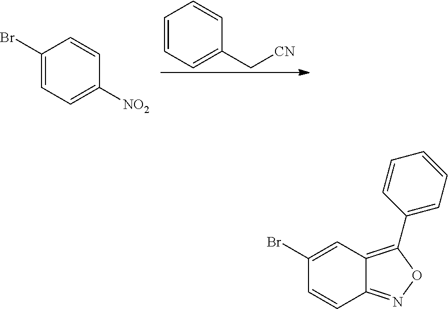

To a vigorously stirred solution of potassium hydroxide (111.0 g, 1.98 mol) in anhydrous methanol (400 mL), phenyl acetonitrile (11.40 g, 99.31 mmol) was added and cooled to 0° C. in ice bath. To this pale yellow color solution, a solution of 1-bromo-4-nitrobenzene (20.0 g, 99.0 mmol) in a mixture of anhydrous methanol (80 mL) and anhydrous tetrahydrofuran (120 mL) was added dropwise, while maintaining the temperature at 0° C. (ice bath). The reaction mixture turned blue on addition of phenyl acetonitrile. The reaction was stirred at 0° C. for 3 h followed by at rt for 3 h and finally refluxed for overnight. On refluxing, the reaction turned dark violet in color. This dark violet colored solution was poured into a mixture of water and crushed ice, stirred well and the violet precipitate was filtered under suction. The residue was washed with water until it became off-white in color and the filtrate became colorless, dried well under reduced pressure to obtain 5-bromo-3-phenyl-benzo[c]isooxazole (20.0 g, 73.6%). Mp 114-115° C. ¹H NMR (400 MHz, CDCl₃): δ 7.37 (dd, J=11.1, 1.4 Hz, 1H), 7.49-7.69 (m, 4H), 7.95-7.99 (m, 2H), 8.03 (s, 1H).

### Preparation of 2-Amino-5-bromo benzophenone

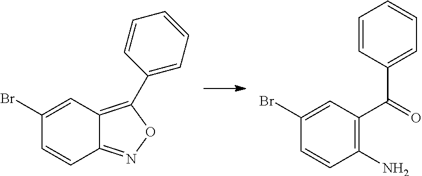

To a hot (80-100° C.) solution of the 5-bromo-3-phenyl-benzo[c]isooxazole (20.0 g, 73.0 mmol) in glacial acetic acid (550 mL), iron powder (45.0 g, 802.0 mmol) and water (275 mL) were added in portions for a period of 2 h. After heating for 3 h, the brown solution was poured into a mixture of water and crushed ice, stirred well, the golden yellow precipitate was filtered under suction, washed with water until the washings became colorless and dried under reduced pressure to obtain 2-amino-5-bromo-benzophenone (19.50 g, 94%) as a golden yellow solid, Mp 112-113° C. ¹H NMR (400 MHz, CDCl₃): δ 5.90-6.25 (br s, 2H, D₂O exchangeable), 6.72 (d, J=8.80 Hz, 1H), 7.37 (dd, J=11.7, 2.3 Hz, 1H), 7.47 (t, J=7.8 Hz, 2H), 7.51-7.58 (m, 2H), 7.59-7.64 (m, 2H).

### Preparation of 6-Bromo-2-chloro-4-phenyl-quinoline-3-carbonyl chloride

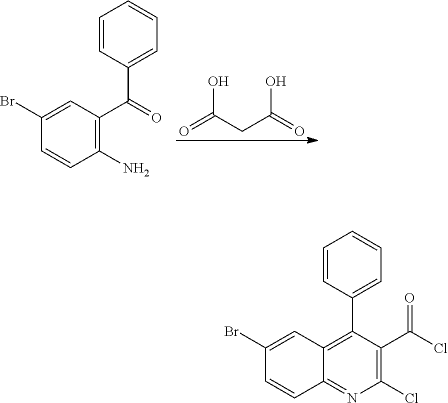

2-amino-5-bromo-benzophenone (10.0 g, 36.23 mmol) and malonic acid (5.65 g, 54.30 mmol) were mixed, dried under reduced pressure, dissolved in freshly distilled phosphorous oxychloride (200 mL) and heated at 105° C. for 3 h. The brown solution was poured into crushed ice in portions with constant shaking and extracted with dichloromethane (2×500 mL). The dichloromethane extract was washed with water until the aqueous layer became neutral to pH paper followed by brine (1×100 mL), dried over anhydrous sodium sulfate, filtered and the dichloromethane was evaporated under reduced pressure to obtain a brown gum. Purification of this gum by column chromatography (silica gel 100-200 mesh, gradual elution n-hexane to 3% ethyl acetate in n-hexane) gave 6-bromo-2-chloro-4-phenyl-quinoline-3-carbonyl chloride (8.50 g, 61.5%) as an off white solid, Mp 166-170° C. ¹H NMR (400 MHz, CDCl₃): δ 7.34-7.40 (m, 2H), 7.52-7.61 (m, 3H), 7.74 (d, J=2.0 Hz, 1H), 7.89 (dd, J=7.0, 2.0 Hz, 1H), 7.97 (d, J=8.9 Hz, 1H). [M+H]⁺=382, 384.

### Preparation of 2-Bromo-6-chloro-indeno[2,1-c]quinolin-7-one

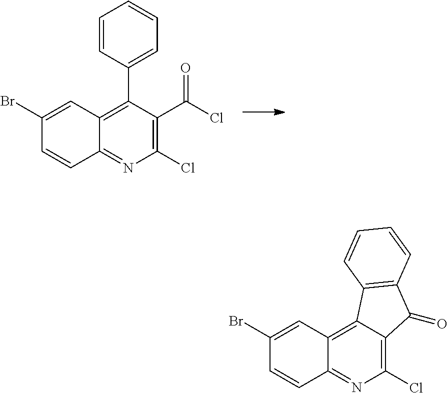

To a solution of the 6-Bromo-2-chloro-4-phenyl-quinoline-3-carbonyl chloride (8.15 g, 21.39 mmol) in dichloromethane (150 mL), aluminum chloride (11.41 g, 85.57 mmol) was added and the mixture was stirred at room temperature for 3 h. The solution turned brown in color. This brown solution was cooled in ice bath, ice pieces were added to quench the reaction and stirred vigorously for about 1 h. The product formed the yellow suspension and was extracted with dichloromethane (4×500 mL), the yellow solid obtained after evaporation of the dichloromethane was washed with methanol (3×100 mL), ethyl acetate (2×50 mL) and n-hexane (2×50 mL) and dried under reduced pressure to obtain 2-bromo-6-chloro-indeno [2,1-c]quinolin-7-one (6.20 g, 84%) as a yellow solid, Mp 304-306° C. ¹H NMR (400 MHz, CDCl₃): δ 7.56 (t, J=7.5 Hz, 1H), 7.68 (dt, J=7.6, 1.2 Hz, 1H), 7.83 (d, J=7.1 Hz, 1H), 7.89-7.98 (m, 2H), 8.11 (d, J=7.6 Hz, 1H), 8.64 (d, J=1.4 Hz, 1H). [M+H]⁺=344, 346.

### Preparation of 2-Bromo-6-methoxy-indeno[2,1-c]quinolin-7-one

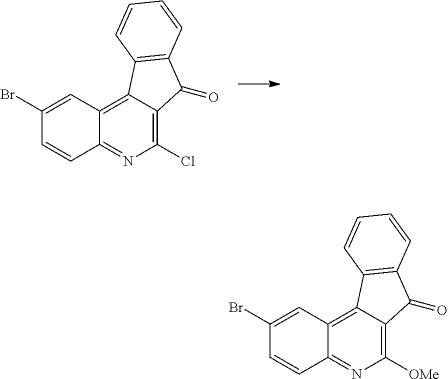

To a suspension of 2-bromo-6-chloro-indeno[2,1-c]quinolin-7-one (5.0 g, 14.51 mmol) in a mixture of anhydrous tetrahydrofuran (300 mL) and anhydrous methanol (150 mL), sodium methoxide (30% w/v in methanol, 26.13 mL, 145.13 mmol) was added and the mixture was refluxed under nitrogen atmosphere for 3 h. The solvents were removed from the brown solution, the brown solid obtained was dissolved in dichloromethane (500 mL), washed with water (3×200 mL) followed by brine (1×100 mL), dried over anhydrous sodium sulfate, filtered and dichloromethane was evaporated under reduced pressure to obtain 2-bromo-6-methoxy-indeno[2,1-c]quinolin-7-one (4.80 g, 97%) as a yellow solid, Mp 208-210° C. ¹H NMR (400 MHz, CDCl₃): δ 4.18 (s, 3H), 7.46 (dt, J=7.4, 0.6 Hz, 1H), 7.58 (dt, J=7.6, 1.2 Hz, 1H), 7.67-7.74 (m, 2H), 7.76 (dd, J=9.0, 2.1 Hz, 1H), 7.96 (d, J=7.6 Hz, 1H), 8.43 (d, J=1.9 Hz, 1H). [M+H]⁺=340, 342.

### Preparation of 2-Bromo-6-methoxy-7-methyl-7H-indeno[2,1-c]quinolin-7-ol

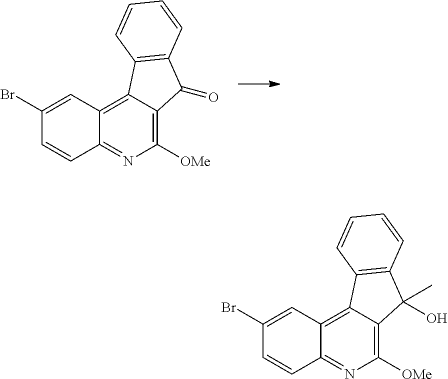

To a solution of 2-Bromo-6-methoxy-indeno[2,1-c]quinolin-7-one (2.0 g, 5.9 mmol) in anhydrous tetrahydrofuran (130 mL), freshly prepared methyl magnesium iodide (1 M solution in diethyl ether, 7.1 mL, 7.1 mmol) was added in one portion at 20° C. under nitrogen atmosphere and the solution was stirred for 3 h allowing it to gradually warm up to rt during which the color of the solution changed from yellow to dark brown. Quenching was done by addition of ice pieces; the reaction was diluted with ethyl acetate (150 mL), washed with saturated ammonium chloride solution (60 mL), water (100 mL) and brine (50 mL). The organic extract was dried over anhydrous sodium sulfate, filtered and the solvents were evaporated under reduced pressure to obtain a brown sticky mass. Purification by column chromatography (silica gel 100-200 mesh, eluted with 10% ethyl acetate in n-hexane) gave 2-bromo-6-methoxy-7-methyl-7H-indeno[2,1-c]quinolin-7-ol (1.6 g, 76.5%) as a off white solid, Mp. 159-160° C. ¹H NMR (400 MHz, CDCl₃): δ 1.82 (s, 3H), 4.19 (s, 3H), 7.45-7.53 (m, 2H), 7.62 (dd, J=8.9, 2.0 Hz, 1H), 7.64-7.68 (m, 1H), 7.70 (d, J=8.9 Hz, 1H), 8.04-8.10 (m, 1H), 8.54 (d, J=2.0 Hz, 1H). [M+H]⁺=356, 358.

### Preparation of 2-Bromo-6-methoxy-7-methyl-7-oxiranylmethoxy-7H-indeno[2,1-c]quinoline

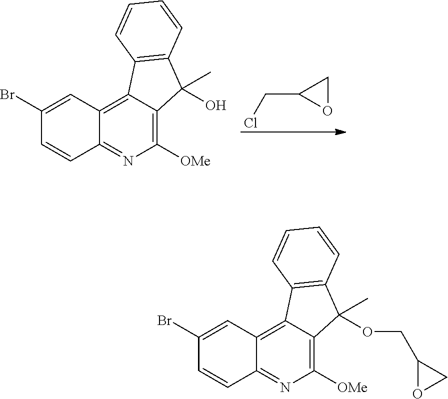

To a cooled solution 2-Bromo-6-methoxy-7-methyl-7H-indeno[2,1-c]quinolin-7-ol (0° C., ice bath) of (2.0 g, 5.62 mmol) in anhydrous N,N-dimethylformamide (7 mL) under nitrogen atmosphere, sodium hydride (0.28 g, 11.8 mmol) was added and stirred for 30 min. During this period, the color of the solution changed from yellow to dark red with evolution of hydrogen gas. epi-chlorohydrin (1.1 g, 11.8 mmol) was added to the reaction mixture and stirring was continued for 48 h at rt before it was quenched with ice pieces. The reaction was diluted with ethyl acetate, washed with brine (3×50 mL), dried over anhydrous sodium sulfate, filtered and the solvents were evaporated under reduced pressure to obtain a gum. Purification by column chromatography (silica gel 100-200 mesh, eluent 8% ethyl acetate in n-hexane) gave 2-bromo-6-methoxy-7-methyl-7-oxiranylmethoxy-7H-indeno[2,1-c]quinolin (1.60 g, 69.5%) as a solid with light yellowish green tingle along with recovery of starting alcohol (0.40 g, 20%), Mp 159-160° C. ¹H NMR (400 MHz, CDCl₃): δ 1.80 (s, 3H), 2.24-2.37 (m, 1H), 2.61 (dd, J=9.4, 5.3 Hz, 1H), 2.76-2.91 (m, 1H), 2.92-3.04 (m, 2H), 4.18 (s, 3H), 7.45-7.56 (m, 2H), 7.59-7.66 (m, 1H), 7.73 (dd, J=8.8, 1.6 Hz, 1H), 7.82 (dd, J=9.0, 1.6 Hz, 1H), 8.17 (d, J=7.0 Hz, 1H), 8.62 (s, 1H). [M+H]⁺=412, 414.

### Preparation of 1-Azido-3-(2-bromo-6-methoxy-7-methyl-7H-indeno[2,1-c]quinolin-7-yloxy)-propan-2-al

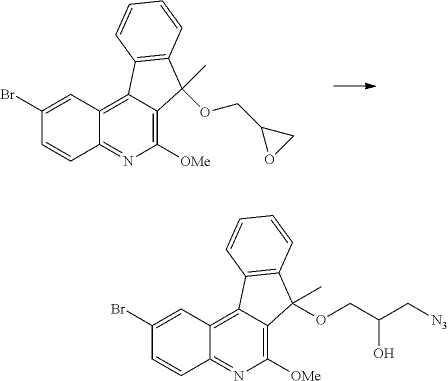

2-bromo-6-methoxy-7-methyl-7-oxiranylmethoxy-7H-indeno[2,1-c]quinolin (0.05 g, 0.12 mmol), ammonium chloride (0.02 g, 0.61 mmol), sodium azide (0.04 g, 0.61 mmol) were dissolved in a mixture of methanol and water (8:1) and the mixture was heated at 70-95° C. for 10 h. The solvents were evaporated under reduced pressure, the solid obtained was dissolved in ethyl acetate (10 mL) and washed with water (2×5 mL) followed by brine (5 mL). The organic layer was dried over anhydrous sodium sulfate, filtered and the solvents were evaporated to obtain a sticky mass, which on purification by flash chromatography (silica gel 100-200 mesh, eluted with 10% ethyl acetate in n-hexane) gave 1-Azido-3-(2-bromo-6-methoxy-7-methyl-7H-indeno[2,1-c]quinolin-7-yloxy)-propan-2-ol (0.04 g, 80%) as a sticky mass. ¹H NMR (400 MHz, CDCl₃): δ 1.81 (s), 2.53-2.60 (m), 2.71-2.79 (m), 2.95-3.05 (m), 3.05-3.15 (m), 3.25-3.33 (m), 3.59-3.65 (m), 3.80-3.90 (m), 4.19 (s), 7.45-7.59 (m), 7.73-7.77 (m), 7.82-7.88 (m), 8.16-8.21 (m), 8.63 (s) total 1811 in a diastereomeric ratio 1:1. [M+H]⁺=455, 457.

### Preparation of 1-Azido-3-(2-bromo-6-methoxy-7-methyl-7H-indeno[2,1-c]quinolin-7-yloxy)-propan-2-ol

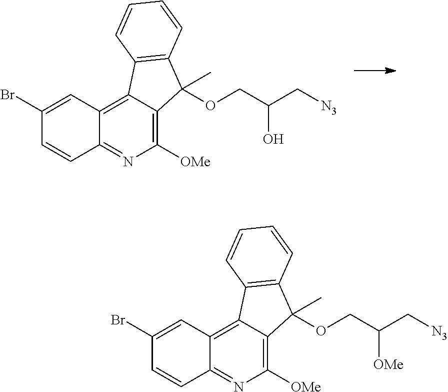

1-Azido-3-(2-bromo-6-methoxy-7-methyl-7H-indeno[2,1-c]quinolin-7-yloxy)-propan-2-ol (0.94 g, 2.06 mmol) and methyl iodide (0.29 g, 2.06 mmol) were dissolved in anhydrous N,N dimethylformamide (10 mL) and the mixture was cooled to 0° C. To this mixture sodium hydride (0.05 g, 2.06 mmol) was added and the reaction was stirred for 2 h. The reaction was quenched with ice pieces, diluted with ethyl acetate (30 mL), washed with brine (2×25 mL), the organic layer was dried over anhydrous sodium sulfate, filtered and the solvents were evaporated to obtain 1-azido-3-(2-bromo-6-methoxy-7-methyl-7H-indeno[2,1-c]quinolin-7-yloxy)-propan-2-ol (0.77 g, 80%) as a sticky mass. ¹H NMR (400 MHz, CDCl₃): δ 1.84 (s), 2.68-2.78 (m), 3.25 (s), 3.27 (s), 3.28-3.37 (m), 4.17 (s), 4.18 (s), 7.47-7.56 (m), 7.56-7.60 (m), 7.73 (dd, J=8.9, 2.0 Hz), 7.82 (d, J=9.0 Hz), 8.0 (s), 8.18 (d, J=7.6 Hz), 8.63 (s) total 21H in a diastereomeric ratio 1:1. [M+H]⁺=470, 472.

### Preparation of 2-Bromo-6-methoxy-7H-indeno[2,1-c]quinoline

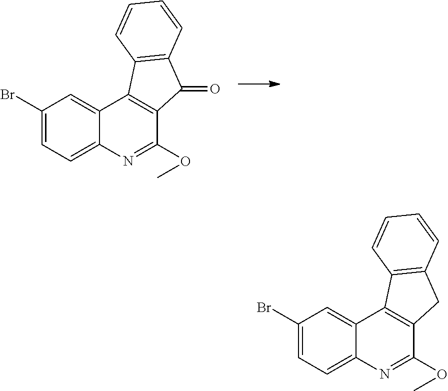

A suspension of 2-bromo-6-methoxy-indeno[2,1-c]quinolin-7-one (2.40 g, 7.05 mmol) in a mixture of hydrazine hydrate (18.50 g, 370.35 mmol) and 1,2-ethane diol (80 mL) was heated at 140° C. and the temperature was gradually increased to 180° C. during 3.5 h. The reaction was then poured into a mixture of crushed ice and water, stirred well, extracted with dichloromethane (3×100 mL) and washed with brine (2×50 mL). The organic extract was dried over anhydrous sodium sulfate, filtered and the solvents were evaporated under reduced pressure to obtain a solid, which on purification by column chromatography gave pure 2-bromo-6-methoxy-7H-indeno[2,1-c]quinoline (1.819 g, 79%) as a white fluffy solid, Mp 150-152° C. ¹H NMR (400 MHz, CDCl₃): δ 3.89 (s, 2H), 4.16 (s, 3H), 7.46 (dt, J=7.4, 1.2 Hz, 1H), 7.52 (t, J=7.5 Hz, 1H), 7.66 (d, J=7.4 Hz, 1H), 7.69 (dd, J=7.8, 2.2 Hz, 1H), 7.83 (d, J=8.9 Hz, 1H), 8.25 (d, J=7.6 Hz, 1H), 8.63 (d, J=2.0 Hz, 1H). [M+H]⁺=326, 328.

### Preparation of 2-Bromo-6-imidazol-1-yl-indeno[2,1-c]quinolin-7-one

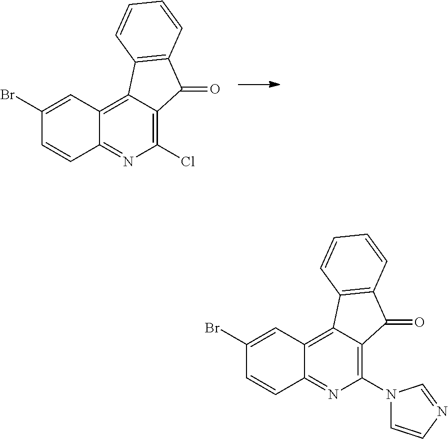

A mixture of 2-bromo-6-chloro-indeno[2,1-c]quinolin-7-one (0.50 g, 1.44 mmol) and the imidazole (0.40 g, 7.24 mmol) were heated in anhydrous pyridine (10 mL) at 105° C. for 12 h. the reaction was cooled to room temperature, poured into water, the precipitate obtained was filtered, washed with water and dried under reduced pressure to obtain 2-Bromo-6-imidazol-1-yl-indeno[2,1-c]quinolin-7-one (0.302, 87%) as a red solid, Mp 283-285° C. ¹H NMR (400 MHz, CD₃OD+DMSO-d₆): δ 7.60-7.73 (m, 3H), 7.82-7.86 (m, 1H), 8.06-8.10 (m, 1H), 8.14 (d, J=8.8 Hz, 1H), 8.22 (s, 1H), 8.44 (d, J=8.0 Hz, 1H), 8.94 (s, 1H), 9.40 (s, 1H). [M+H]⁺=376, 378.

### Preparation of 2-Bromo-6-(4-pyridin-2-yl-piperazin-1-yl)-indeno[2,1-c]quinolin-7-one

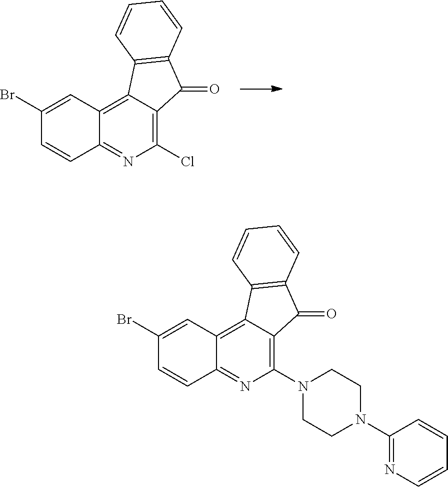

A mixture of 2-bromo-6-chloro-indeno[2,1-c]quinolin-7-one (0.5 g, 1.44 mmol) and the 1-(2-Pyridyl) piperizine (1.18 g, 7.20 mmol) were heated in anhydrous pyridine (20 mL) at 105° C. for 12 h. the reaction was cooled to rt, poured into water, the precipitate obtained was filtered, washed with water and dried under reduced pressure to obtain the corresponding 2-Bromo-6-(4-pyridin-2-yl-piperazin-1-yl)-indeno[2,1-c]quinolin-7-one (0.624 g, 92%) as a red solid, Mp 186-188° C. ¹H NMR (400 MHz, CDCl₃): δ 3.79 (s, 8H), 6.63-6.66 (m, 1H), 6.71 (d, J=8.4 Hz, 1H), 7.44-7.53 (m, 2H), 7.56-7.60 (m, 1H), 7.64-7.73 (m, 3H), 8.01 (d, J=7.6 Hz, 1H), 8.22 (d, J=3.2 Hz, 1H), 8.45 (d, J=1.6 Hz, 1H). [M+H]⁺=471, 473.

### Preparation of 6-Bromo-2,4-dichloro-quinoline-3-carboxylic acid ethyl ester

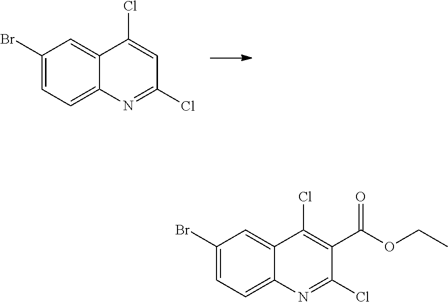

To the cooled solution (−20° C.) of LDA (DTPA, 6.6 ml, 49 mmol; n-BuLi, 27.07 mL, 43 mmol) in dry THF (40 mL) the compound 6-Bromo-2,4-dichloro quinoline (10 g, 36.10 mmol) in dry THF (200 mL) was added dropwise, changing reaction colour to reddish brown and stirred at −78° C. for 40 min. After the anion formation ethylchloroformate (4.14 mL, 43.32 mmol) was added. Reaction was stirred at −78° C. for 2 h and quenched by ice cold water. Reaction mixture was concentrated on rotatory evaporator, and extracted with ethyl acetate (200 mL×3 times). The combined organic layer was washed with brine. The crude product was purified by column chromatography (silica gel 100-200 mesh, 2-3% ethyl acetate in n-hexane) to get 6-Bromo-2,4-dichloro-quinoline-3-carboxylic acid ethyl ester (8.5 g, 67%) as white solid. Mp 120-121° C. ¹H NMR (CDCl₃, 400 MHz): δ 1.44 (t, J=7 Hz, 3H), 4.52 (q, J=7 Hz, 2H), 7.90 (d, J=1 Hz, 2H), 8.37 (s, 1H).

### Preparation of 6-Bromo-4-chloro-2-phenylamino-quinoline-3-carboxylic acid ethyl ester

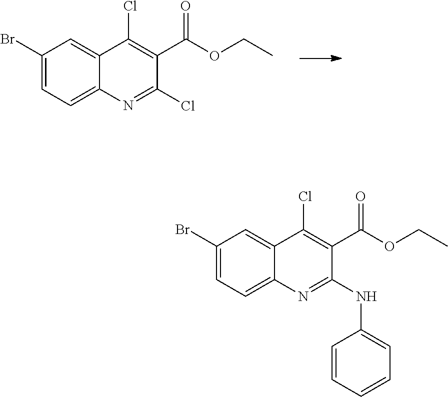

6-Bromo-2,4-dichloro-quinoline-3-carboxylic acid ethyl ester (5.0 g, 14.36 mmol), aniline (3.1 mL, 34.5 mmol) and potassium carbonate (6.0 g, 43.1 mmol) were heated at 100° C., in presence of dry DMF for 14 h. Reaction was quenched with water, extracted with ethyl acetate (50 mL×2), washed with water, brine and dried over sodium sulphate. Organic layer was concentrated under vacuum to get crude product. Crude product was purified by column chromatography (silica gel 100-200 mesh, 6% ethyl acetate in hexane) to get 6-Bromo-4-chloro-2-phenylamino-quinoline-3-carboxylic acid ethyl ester (4.0 g, 68%) as pale yellow solid. Mp 171-172° C. ¹H NMR (CDCl₃, 400 MHz): δ 1.34 (t, J=7.2 Hz, 3H), 4.24 (q, J=7.2 Hz, 2H), 6.98 (d, J=7.8 Hz, 2H), 7.12-7.16 (m, 1H), 7.30 (t, J=7.7 Hz, 2H), 7.71 (dd, J=8.9, 2 Hz, 1H), 7.77 (d, J=8.9 Hz, 1H), 7.81 (d, J=2 Hz, 1H), 8.09 (s, 1H, D₂O exchangeable).

### Preparation of 6-Bromo-4-chloro-2-phenylamino-quinoline-3-carboxylic acid

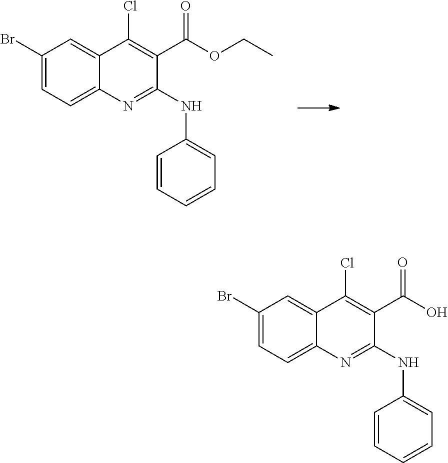

6-Bromo-4-chloro-2-phenylamino-quinoline-3-carboxylic acid ethyl ester (5.0 g, 12.34 mmol) was dissolved in ethanol (50 mL) in presence of sodium hydroxide (20% aq. 70 mL) and stirred at room temperature for 16 h. Reaction was neutralized with dilute hydrochloric acid, and extracted with ethyl acetate (60 mL×3), dried over sodium sulphate and concentrated under vacuum to get crude product. Crude product on n-pentane wash gave pure 6-Bromo-4-chloro-2-phenylamino-quinoline-3-carboxylic acid (3.5 g, 70%) as yellow solid. ¹H NMR (CDCl₃, 400 MHz): δ 7.06 (t, J=8.5 Hz, 3H), 7.27 (t, J=8 Hz, 2H), 7.79 (d, J=8 Hz, 1H), 7.92 (dd, J=9, 2 Hz, 1H), 8.50 (d, J=2 Hz, 1H), 9.20 (s, 1H, D₂O exchangeable), 13.21 (bs, 1H, D₂O exchangeable).

### Preparation of 2-Bromo-12-chloro-dibenzo[b,g][1,8]naphthyridin-11-ol

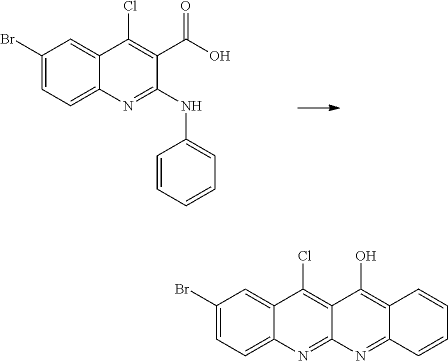

Chlorosulphonic acid (4 mL, 59.7 mmol) was added to 6-Bromo-4-chloro-2-phenylamino-quinoline-3-carboxylic acid (0.400 g, 1.06 mmol) at 0° C. and was stirred for 2 h. Reaction was allowed to come to room temperature and dry dichloromethane (2.5 mL), phosphorus pentaoxide (0.100 g 0.35 mmol) was added to it and stirred for 12 h. Reaction was quenched with ice, neutralized with sodium bicarbonate, extracted with dichloromethane (25 mL×4), washed with brine and dried over sodium sulphate. Organic layer was concentrated under vacuum to get crude product. Crude product was purified by column chromatography (silica gel 100-200 mesh, 30% ethyl acetate in hexane) to get 2-Bromo-12-chloro-dibenzo[b,g][1,8]naphthyridin-1′-ol (0.1 g, 40%) as a muddy colored solid. ¹H NMR (DMSO-d₆, 400 MHz): 7.46 (t, J=7.3 Hz, 1H), 7.80-7.92 (m, 2H), 7.96-8.01 (m, 1H), 8.03-8.13 (m, 1H), 8.26 (d, J=7.6 Hz, 1H), 9.2 (s, 1H), 12.05 (s, 1H, D₂O exchangeable).

### Preparation of 2-(2-Bromo-12-chloro-dibenzo[b,g][1,8]naphthyridin-11-yloxy)-1-imidazol-1-yl-ethanol

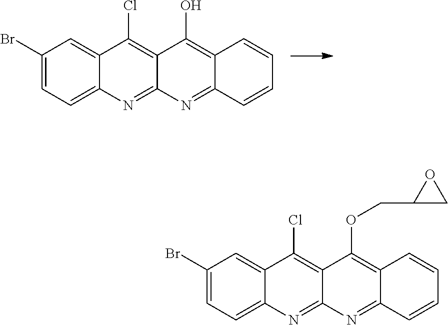

2-Bromo-12-chloro-dibenzo[b,g][1,8]naphthyridin-11-ol (0.050 g, 0.14 mmol) was dissolved in acetonitrile (2.5 mL) and heated to 90° C. for 15 min. Then cesium carbonate (0.135 g, 0.417 mmol), and tetra-butyl-ammonium bromide (0.01 g, 0.031 mmol) was added and stirred for 30 min followed by addition of epi-chlorohydrin (0.03 mL, 0.418 mmol) for 10 h. Reaction was quenched by water, extracted with ethyl acetate (20 mL×2), washed with water, brine and dried over sodium sulphate. Organic layer was concentrated under vacuum to get crude product. Crude product was purified by column chromatography (silica gel, 15% ethyl acetate in hexane) to get 2-(2-Bromo-12-chloro-dibenzo[b,g][1,8]naphthyridin-11-yloxy)-1-imidazol-1-yl-ethanol (0.025 g, 40%) as a sticky product. ¹H NMR (DMSO-d₆, 400 MHz): 2.67 (dd, J=4.8, 2.5 Hz, 1H), 2.84-2.93 (m, 1H), 3.18-3.29 (m, 1H), 3.55 (d, J=5.4 Hz, 2H), 7.46 (t, J=7.3 Hz, 1H), 7.80-7.92 (m, 2H), 7.96-8.01 (m, 1H), 8.03-8.13 (m, 1H), 8.26 (d, J=7.6 Hz, 1H), 9.2 (s, 1H).

### Preparation of 2-Benzylamino-6-bromo-4-chloro-quinoline-3-carboxylic acid ethyl ester

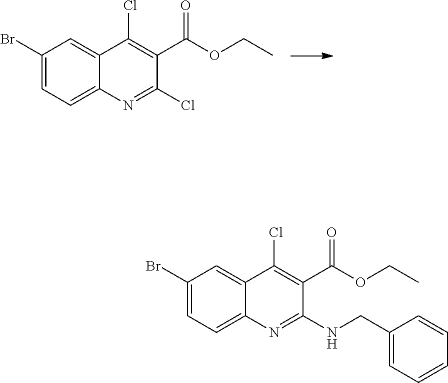

6-Bromo-2,4-dichloro-quinoline-3-carboxylic acid ethyl ester (10 g, 28.65 mmol) and benzylamine (4.7 mL, 43 mmol) were dissolved in dry toluene (200 mL) and heated at 100° C. under nitrogen atmosphere, for 15 h. Reaction was allowed to come to room temperature and basified by sodium carbonate and extracted with ethyl acetate (250 mL×3). Ethyl acetate layer was washed with brine and dried over sodium sulphate and concentrated to get yellowish solid as a crude product. Crude product was purified by column chromatography (silica gel 100-200 mesh, 5-6% ethyl acetate in hexane) to get 2-Benzylamino-6-bromo-4-chloro-quinoline-3-carboxylic acid ethyl ester (8.5 g, 71%) as an off-white solid. Mp 163-165° C. ¹H NMR (CDCl₃, 400 MHz): δ 1.36 (t, J=7 Hz, 3H), 4.36 (q, J=7 Hz, 2H), 4.57 (d, J=5 Hz, 2H), 5.87 (s, 1H, D₂O exchangeable), 7.31-7.46 (m, 5H), 7.71 (dd, J=9, 2 Hz, 1H), 7.74 (d, J=9 Hz, 1H), 7.92 (d, J=2 Hz, 1H).

### Preparation of 2-Benzylamino-6-bromo-4-chloro-quinoline-3-carboxylic acid

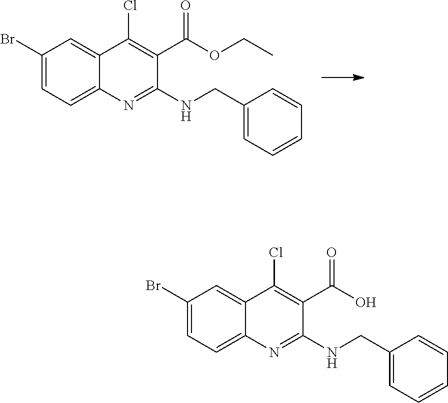

2-Benzylamino-6-bromo-4-chloro-quinoline-3-carboxylic acid ethyl ester (4.5 g, 10.7 mmol), was dissolved in ethanol:THF (3:1, 100 mL) and stirred in presence sodium hydroxide (20% aq. 25 mL), at room temperature for 14 h. Reaction mixture was acidified with 3N HCl, extracted with ethyl acetate (200 mL×2 times), dried over sodium sulphate, and concentrated under vacuum to get crude mixture. Crude mixture was purified by n-pentane washes, to get 2-Benzylamino-6-bromo-4-chloro-quinoline-3-carboxylic acid (4 g, 95%), as brown solid. Mp 182-184° C. ¹H NMR (CDCl₃, 400 MHz): δ 4.61 (d, J=6 Hz, 2H), 7.22-7.38 (m, 5H), 7.68 (d, J=9 Hz, 1H), 7.83 (dd, J=9, 2 Hz, 1H), 7.93 (t, J=6 Hz, 1H, D₂O exchangeable), 8.72 (d, J=2 Hz, 1H), 13.71 (s, 1H, D₂O exchangeable).

### Preparation of 8-Bromo-6-chloro-12,13-dihydro-11,12-diaza-benzo[4,5]cyclohepta[1,2-b]naphthalen-5-one

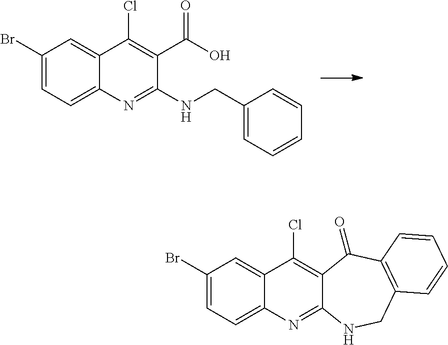

2-Benzylamino-6-bromo-4-chloro-quinoline-3-carboxylic acid (0.500 g, 1.27 mmol) and thionyl chloride (5 mL) was refluxed for 3 h. Reaction mixture was concentrated on rotatory evaporator, co-evaporated with benzene (10 mL×3) and flushed with nitrogen. This was dissolved in dry dichloromethane, and aluminium trichloride (0.508 g, 3.81 mmol) was added to it at 0° C. under nitrogen atmosphere. Reaction was stirred at 0° C. temperature for 2 h, and quenched by adding ice. Reaction mixture was extracted with ethyl acetate (250 mL×3), dried over sodium sulphate and concentrated on rotatory evaporator to get crude product. Crude product was purified by column chromatography (neutral aluminium oxide, 30% ethyl acetate in hexane), to get 8-Bromo-6-chloro-12,13-dihydro-11,12-diaza-benzo[4,5]cyclohepta[1,2-b]naphthalen-5-one as a pale yellow solid (0.190 g, 40%). Mp 260-264° C. ¹H NMR (CDCl₃, 400 MHz): δ 4.47 (d, J=5 Hz, 2H), 7.40-7.45 (m, 3H), 7.54-7.58 (m, 1H), 7.64 (d, J=9 Hz, 1H), 7.87 (dd, J=9, 2 Hz, 1H), 8.5 (d, J=2 Hz, 1H), 9.2 (t, J=5 Hz, 1H, D₂O exchangeable).

### Preparation of 8-Bromo-6-chloro-12-methyl-12,13-dihydro-11,12-diaza-benzo[4,5]cyclohepta[1,2-b]naphthalen-5-one

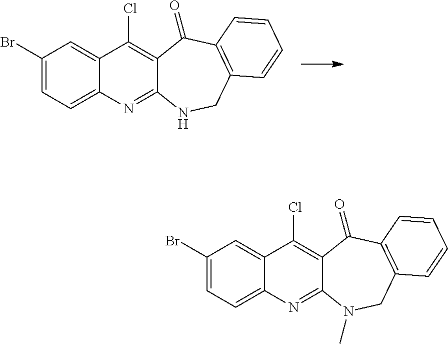

8-Bromo-6-chloro-12,13-dihydro-11,12-diaza-benzo[4,5]cyclohepta[1,2-b]naphthalen-5-one (2.0 g, 5.36 mmol) was dissolved in dry DMF (100 mL), sodium hydride (0.257 g, 10.72 mmol) was added to it and stirred for 15 min at 0° C., followed by addition of methyl iodide (0.67 mL, 10.72 mmol). Reaction was stirred for 2 h at room temperature, quenched by ice and extracted with ethyl acetate (100 mL×3).

Organic layer was washed with brine, dried over sodium sulphate and concentrated on rotatory evaporator to get crude solid. Crude compound was purified by column chromatography (silica gel 100-200 mesh, 20% ethyl acetate in hexane) to get 8-Bromo-6-chloro-12-methyl-12,13-dihydro-11,12-diaza-benzo[4,5]cyclohepta[1,2-b]naphthalen-5-one as yellow solid (1 g, 50%). Mp 153-155° C. ¹H NMR (CDCl₃, 400 MHz): δ 3.12 (s, 3H), 4.54 (s, 2H), 7.31 (d, J=7 Hz, 1H), 7.47 (t, J=7 Hz, 1H), 7.55 (td, J=7.4, 1 Hz, 1H), 7.77 (s, 2H), 7.95 (d, J=7 Hz, 1H), 8.26 (s, 1H).

### Preparation of 8-Bromo-6-chloro-12-methyl-12,13-dihydro-5H11,12diazabenzo[4,5]cyclohepta[1,2-b]naphthalen-5-ol

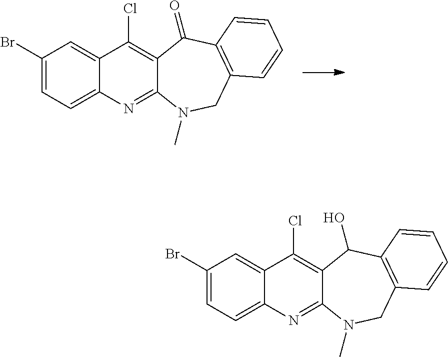

8-Bromo-6-chloro-12-methyl-12,13-dihydro-11,12-diaza-benzo[4,5]cyclohepta[1,2-b]naphthalen-5-one (1 g, 2.6 mmol) was dissolved in THF:MeOH (2:3, 10 mL) and cooled at 0° C. followed by addition of sodium borohydride (0.49 g, 0.013 mmol) Reaction was stirred at room temperature for 3 h, quenched by ice and reaction mixture was concentrated under vacuum. Crude mixture was extracted with ethyl acetate (50 mL×3), and purified by column chromatography (silica gel 100-200 mesh, 20% ethyl acetate in hexane) to get pure 8-Bromo-6-chloro-12-methyl-2,13-dihydro-5H11,12 diazabenzo[4,5]cyclohepta[1,2-b]naphthalen-5-ol (0.85 g, 85%), as an off-white solid. Mp 192-194° C. ¹H NMR (CDCl₃, 400 MHz): δ 2.88 (s, 3H), 3.94 (d, J=14 Hz, 1H), 5.55 (d, J=14 Hz, 1H), 6.30 (s, 1H, D₂O exchangeable), 7.28-7.44 (m, 4H), 7.67 (dd, J=2, 9 Hz, 1H), 7.72 (d, J=9 Hz, 1H), 8.20 (d, J=2 Hz, 1H).

### Preparation of 8-Bromo-6-chloro-12-methyl-5-oxiranylmethoxy-12,13-dihydro-5H-11,12-diaza-benzo[4,5]cyclohepta[1,2-b]naphthalene

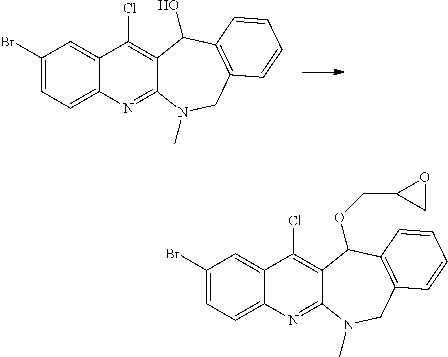

8-Bromo-6-chloro-12-methyl-12,13-dihydro-5H11,12diazabenzo[4,5]cyclohepta[1,2-b]naphthalen-5-ol (1 g, 2.57 mmol), was dissolved in dry THF (100 mL) and epi-chlorohydrine (2 mL, 25.7 mmol) was added at room temperature. Reaction mixture was cooled to 0° C., sodium hydride (0.062 g, 2.57 mmol) and dry DMF (0.1 mL) was added to it. Reaction was stirred at room temperature for 7 h. Reaction mixture was concentrated under vacuum and extracted with ethyl acetate (50 mL×3), followed by brine wash. Organic layer was dried over sodium sulphate and concentrated under vacuum to get crude product. Crude product was purified by column chromatography (silica gel 100-200 mesh, 15% ethyl acetate in hexane) to get 8-Bromo-6-chloro-12-methyl-5-oxiranylmethoxy-12,13-dihydro-5H-11,12-diaza-benzo[4,5]cyclohepta[1,2-b]naphthalene (0.45 g, 40%) as pale yellow gum, ¹H NMR (CDCl₃, 400 MHz): δ 2.42-2.54 (m, 1H), 2.68-2.78 (m, 1H), 2.88 (d, J=2.0 Hz, 3H), 3.07-3.15 (m, 1H), 3.27 (dd, J=11.0, 5.0 Hz, 0.5H), 3.41 (dd, J=10.2, 5.4 Hz, 0.5H), 3.55 (dd, J=11.0, 3.1 Hz, 0.5H), 3.69 (dd, J=10.2, 3.1 Hz, 0.5H), 3.91 (dd, J=14.3, 4.8 Hz, 1H), 5.49 (dd, J=14.3, 2.1 Hz, 1H), 5.84 (s, 0.5H), 5.96 (s, 0.5H), 7.28-7.44 (m, 4H), 7.65 (dd, J=8.8, 2 Hz, 1H), 7.72 (d, J=8.8 Hz, 1H), 8.18 (d, J=2 Hz, 1H).

### Example-8

### Preparation of 2-Bromo-6-imidazol-1-yl-7-methyl-7H-indeno[2,1-c]quinolin-7-ol

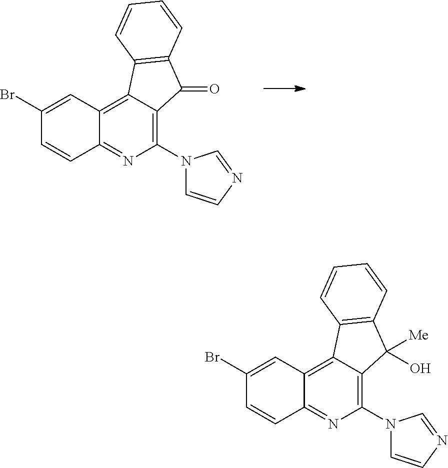

Freshly prepared methyl magnesium iodide (1 M in diethyl ether, 7.93 mL) was added to a cooled (ca 0° C., ice-bath) tetrahydrofuran (60 mL) solution of 2-Bromo-6-imidazol-1-yl-indeno[2,1-quinolin-7-one (2.00 g, 5.29 mmol) and the reaction was stirred at 0° C. (ice bath) for 30 min. After further stirring at rt for 30 min the reaction was quenched with ice-cold water, diluted with ethyl acetate, washed with saturated ammonium chloride solution followed by brine. The organic extract was dried over anhydrous sodium sulfate, filtered and the solvents were evaporated under reduced pressure to obtain a gum, which on purification by column chromatography gave 2-bromo-6-imidazol-1-yl-7-methyl-7H-indeno[2,1-c]quinolin-7-ol (1.20 g, 56%) as pale-yellow solid, Mp 175-180° C. ¹H NMR (400 MHz, DMSO-d₆): δ 1.41 (s, 3H), 6.45 (s, 1H, D₂O exchangeable,), 7.18 (s, 1H), 7.58-7.63 (m, 2H), 7.73 (d, J=4.0 Hz, 1H), 8.04 (s, 2H), 8.20 (s, 1H), 8.51 (d, J=4.0 Hz, 1H), 8.68 (s, 1H), 8.95 (s, 1H). [M+H]⁺=392, 394.

### Example 9

### Preparation of 2-Bromo-6-imidazol-1-yl-indeno[2,1-c]quinolin-7-one oxime

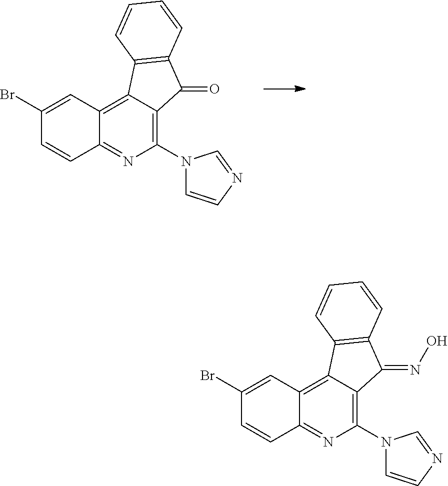

To a cooled (0° C., ice bath) suspension of the 2-bromo-6-imidazol-1-yl-indeno[2,1-quinolin-7-one (0.07 g, 0.17 mmol) and hydroxylamine hydrochloride (0.04 g, 0.53 mmol) in ethanol-water (2:1, v/v) mixture, sodium hydroxide pellets (0.04 g, 0.88 mmol) were added in portions, stirred at 0° C. for 15 min and then heated at 80° C. for 3 h. The reaction was cooled to rt, poured into 15% aqueous solution of hydrochloric acid, the precipitate obtained was filtered, washed with water and dried under reduced pressure to obtain 2-bromo-6-imidazol-1-yl-indeno[2,1-c]quinolin-7-one oxime (0.04 gm, 62%) as a brown solid, Mp 268-271° C. ¹H NMR (400 MHz, DMSO-d₆): δ 7.50-7.54 (m), 7.60-7.64 (m), 7.65-7.80 (m), 7.89 (t, J=8.0 Hz), 7.98 (t, J=8.0 Hz), 8.00-8.05 (m), 8.05-8.13 (m), 8.15 (d, J=4.0 Hz), 8.47-8.53 (m), 8.53-8.60 (m), 8.68-8.75 (m), 8.96 (d, J=8.0 Hz), 9.00 (s), 9.03-9.06 (m), 9.18-9.92 (m). 13.26 (s, D₂O exchangeable), 13.37 (s, D₂O exchangeable) total 11H in a diestereometic ratio 1:1. [M+H]⁺=391, 393.

### Example 10

### Preparation of 2-Bromo-6-(4-pyridin-2-yl-piperazin-1-yl)-indeno[2,1-c]quinolin-7-one-oxime

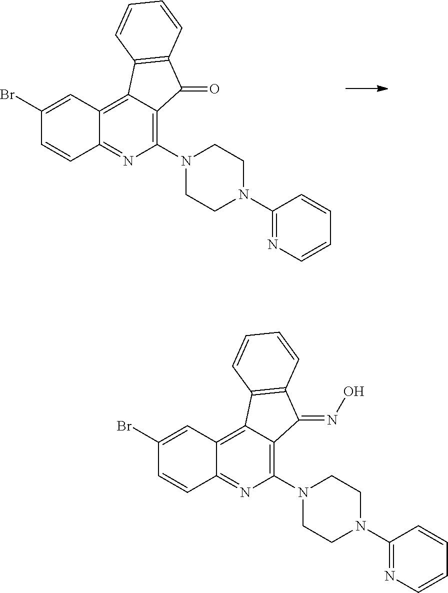

To a cooled (0° C., ice bath) suspension of 2-bromo-6-(4-pyridin-2-yl-piperazin-1-yl)-indeno[2,1-c]quinolin-7-one (0.50 g, 1.06 mmol) and hydroxylamine hydrochloride (0.22 g, 3.18 mmol) in ethanol-water (2:1) mixture, sodium hydroxide pellets (0.13 g, 3.18 mmol) were added in portions, stirred at 0° C. for 15 min and then heated at 80° C. for 3 h. The reaction was cooled to rt, poured into 15% aqueous solution of hydrochloric acid, the precipitate obtained was filtered, washed with water and dried under reduced pressure to obtain 2-bromo-6-(4-pyridin-2-yl-piperazin-1-yl)-indeno[2,1-c]quinolin-7-one-oxime (0.63 g, 96%) as greenish solid, Mp 235-237° C. ¹H NMR (400 MHz, DMSO-dₒ): δ 3.69 (s, 4H), 3.95 (s, 4H), 6.97 (t, J=6.4 Hz, 1H), 7.44 (d, J=8.8 Hz, 1H), 7.59-7.69 (m, 2H), 7.78-7.86 (m, 2H), 8.00-8.07 (m, 2H), 8.49 (d, J=7.2 Hz, 1H), 8.57 (d, J=7.2 Hz, 1H), 8.74 (s, 1H), 13.28 (s, 1H, D₂O exchangeable). [M+H]⁺=486, 488.

### Example 11

### Preparation of 2-bromo-6-(4-pyridin-2-yl-piperazin-1-yl)-indeno[2,1-c]quinolin-7-one N,N-dimethyl carbamoyl-oxime

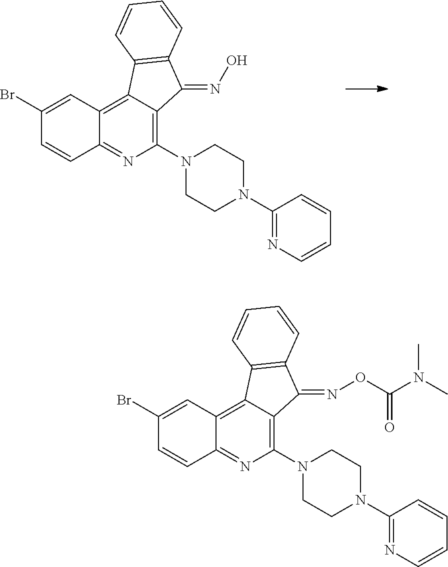

The 2-bromo-6-(4-pyridin-2-yl-piperazin-1-yl)-indeno[2,1-c]quinolin-7-one-oxime (0.10 g, 0.21 mmol) and N,N-dimethylamine carbamoyl chloride (0.04 g, 0.41 mmol) were stirred at rt in anhydrous N,N-dimethylformamide (20 mL) for 12 h. The reaction was poured into water; the precipitate obtained was filtered, washed with cold water and dried under reduced pressure to obtain 2-bromo-6-(4-pyridin-2-yl-piperazin-1-yl)-indeno[2,1-c]quinolin-7-one N,N-dimethyl carbamoyl-oxime (0.05 g, 63%) as a brownish-yellow solid, Mp 215-217° C. ¹H NMR (400 MHz, CDCl₃): δ 3.04 (s, 3H), 3.11 (s, 3H), 3.71-3.85 (m, 5H), 4.05-4.20 (m, 3H), 6.69-6.77 (m, 1H), 6.86-6.94 (m, 1H), 7.48-7.52 (m, 1H), 7.59-7.63 (m, 1H), 7.71-7.79 (m, 3H), 8.21 (d, J=7.6 Hz, 2H), 8.35 (d, J=7.6 Hz, 1H), 8.57 (s, 1H). [M+H]⁺=557, 559.

### Example 12

### Preparation of 1-(2-Bromo-6-methoxy-7-methyl-7H-indeno[2,1-c]quinolin-7-yloxy)-3-[3-(4-trifluoromethyl-phenyl)-pyrazol-1-yl]-propan-2-ol

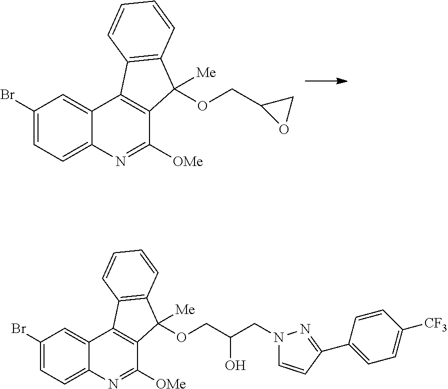

To a mixture of activated potassium carbonate (167.47 g, 1.21 mmol) and compound 2-bromo-6-methoxy-7-methyl-7-oxiranylmethoxy-7H-indeno[2,1-c]quinolin (0.10 g, 0.24 mmol) in anhydrous N,N-dimethylformamide (2 mL), 3-(4-trifluoromethyl-phenyl) pyrazole (0.05 g, 0.24 mmol) was added under nitrogen atmosphere. The mixture was stirred at 65-70° C. for 15 h. The reaction was quenched with ice, diluted with ethyl acetate and washed thrice with brine. The organic extract was dried over anhydrous sodium sulfate, filtered and the solvents were evaporated to obtain an oily stuff which was purified by flash chromatography (neutral alumina, eluted with 10% ethyl acetate in n-hexane) to obtain 1-(2-bromo-6-methoxy-7-methyl-7H-indeno[2,1-c]quinolin-7-yloxy)-3-[3-(4-trifluoromethyl-phenyl)-pyrazol-1-yl]-propan-2-ol (0.07 g, 50%) as a white fluffy-solid, Mp 65-67° C. ¹H NMR (400 MHz, CDCl₃): δ 1.82 (s), 1.84 (s), 2.51 (dd, J=9.6, 7.0 Hz), 2.75 (dd, J=9.5, 5.8 Hz), 2.94 (dd, J=9.5, 4.2 Hz), 3.00 (dd, J=9.6, 4.2 Hz), 3.91-3.98 (m), 3.99-4.02 (m), 4.03-4.11 (m), 4.12-4.15 (m), 4.16 (s), 4.19 (s), 4.22-4.36 (m), 6.30 (d, J=2.4 Hz), 6.53 (d, J=2.2 Hz), 7.38 (d, J=2.2 Hz), 7.43-7.52 (m), 7.53-7.61 (m), 7.64 (d, J=8.2 Hz), 7.70-7.75 (m), 7.77-7.85 (m), 8.09 (d, J=6.3 Hz), 8.15 (d, J=7.1 Hz), 8.52 (d, J=2.0 Hz), 8.60 (d, J=2.0 Hz) for total 25H in diastereomeric ratio 1.4:1. [M+Na]⁺=646, 648.

### Example 13

### Preparation of 1-(2-Bromo-6-methoxy-7H-indeno[2,1-c]quinolin-7-yl)-3-dimethylamino-1-(4-fluoro-phenyl)-propan-1-ol

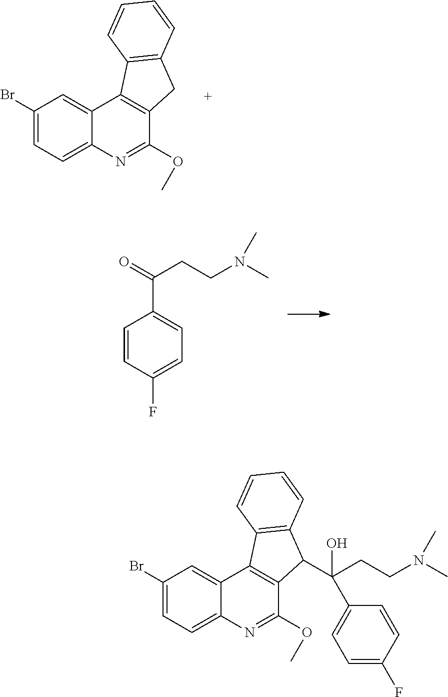

Lithium diisopropyl amide was generated by drop-wise addition of a n-butyl lithium solution (1.6 M in n-hexane, 0.60 mL, 0.96 mmol) into a cooled (−20° C., dry ice-acetone bath) solution of N,N-diisopropyl amine (0.11 g, 1.07 mmol) in anhydrous tetrahydrofuran (4 mL). The mixture was cooled to −78° C. (dry ice-acetone bath), a solution of 2-bromo-6-methoxy-7H-indeno[2,1-c]quinoline (0.10 g, 0.31 mmol) in tetrahydrofuran (3 mL) was added dropwise and stirring continued at −78° C. for 30 min A solution of 3-dimethylamino-1-(4-fluoro-phenyl)-propan-1-one (0.07 g, 0.38 mmol) in tetrahydrofuran (3 mL) was then added drop-wise and stirring continued for overnight. The reaction was diluted with ethyl acetate, washed with brine, concentrated and the solvents were evaporated to obtain a sticky mass. Purification by flash chromatography (silica gel 230-400 mesh, eluted with ethyl acetate n-hexane mixture) gave pure 1-(2-bromo-6-methoxy-7H-indeno[2,1-c]quinolin-7-yl)-3-dimethylamino-1-(4-fluoro-phenyl)-propan-1-ol was obtained (0.01 g, 4%) as a sticky mass. ¹H NMR (400 MHz, CDCl₃): δ 1.83 (t, J=7.3 Hz, 2H), 2.27 (t, J=7.3 Hz, 2H), 2.36 (s, 6H), 3.74 (s, 3H), 4.53 (s, 1H), 5.52 (br s, D₂O exchangeable, 1H), 6.85-6.95 (m, 2H), 7.05-7.25 (m, 5H), 7.40-7.47 (m, 1H), 7.56-7.63 (m, 1H), 8.00-8.10 (m, 1H), 8.12-8.18 (m, 1H). [M+H]⁺=522, 524.

### Example 14

### Preparation of [3-(2-Bromo-6-methoxy-7-methyl-7H-indeno[2,1-c]quinolin-7-yloxy)-2-methoxy-propyl]-(2-methoxy-phenyl)-carbodiimide

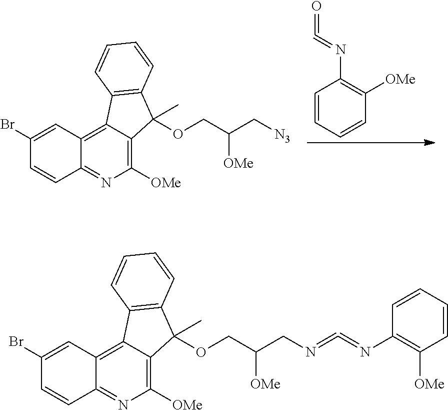

Anhydrous dichloromethane was added to a mixture of 1-azido-3-(2-bromo-6-methoxy-7-methyl-7H-indeno[2,1-c]quinolin-7-yloxy)-propan-2-ol (0.79 g, 1.64 mmol) and triphenyl phosphine (0.440 g, 1.64 mmol) under nitrogen atmosphere at 0° C. and stirred the mixture at rt for 10-12 h. 2-Methoxyphenyl isocyanate (0.276 g, 1.64 mmol) was added drop-wise to the reaction and the reaction was further stirred for 2 h. The solvents were evaporated under reduced pressure, the sticky mass obtained was purified by flash chromatography (silica gel 230-400 mesh, eluent, ethyl acetate-n-hexane mixture) to give pure [3-(2-Bromo-6-methoxy-7-methyl-7H-indeno[2,1-c]quinolin-7-yloxy)-2-methoxy-propyl]-(2-methoxy-phenyl)-carbodiimide (0.16 g, 17%) as a sticky mass. ¹H NMR (400 MHz, CDCl₃): δ 1.81 (s), 2.86-2.95 (m), 3.26 (s), 3.29 (s), 3.30-3.39 (m), 3.40-3.55 (m), 3.75 (s), 3.76 (s), 4.16 (s), 4.17 (s), 6.75-6.84 (m), 6.90-6.95 9 (m), 6.96-7.06 (m), 7.42-7.51 (m), 7.58-7.60 (m), 7.69-7.72 (m), 7.72-7.75 (in), 7.77-7.80 (m), 7.80-7.83 (m), 8.00-8.20 (m), 8.59-8.61 (m) total 28H in a diastereomeric ratio 1:1. [M+H]⁺=574, 576.

### Example 15

### Preparation of 2-(2-Bromo-12-chloro-dibenzo[b,g][1,8]naphthyridin-11-yloxy)-1-imidazol-1-yl-ethanol

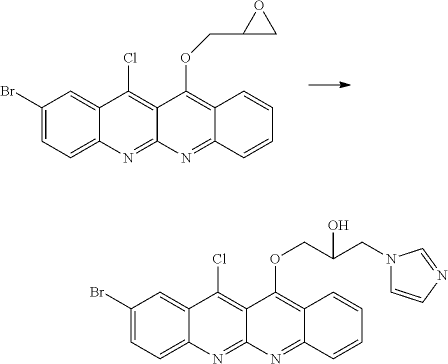

2-(2-Bromo-12-chloro-dibenzo[b,g][1,8]naphthyridin-11-yloxy)-1-imidazol-1-yl-ethanol (0.37 g, 0.9 mmol), potassium carbonate (0.25 g, 1.8 mmol) and imidazole (0.24 g, 3.6 mmol) were refluxed in the presence of isopropanol (20 mL) for 12 h. Reaction mixture was concentrated under vacuum and extracted with ethyl acetate (50 mL×3). Organic layer was washed brine, dried over sodium sulphate and concentrated under vacuum to get crude mixture. Crude mixture was purified by column chromatography (silica gel 100-200 mesh, 5% methanol in dichloromethane), to get 2-(2-Bromo-12-chloro-dibenzo[b,g][1,8]naphthyridin-11-yloxy)-1-imidazol-1-yl-ethanol (0.182 g, 42%) as white solid. ¹H NMR (CDCl₃, 400 MHz): 3.15-3.28 (m, 1H), 3.30-3.42 (m, 1H, D₂O exchangeable), 3.44-3.58 (m, 1H), 3.78-4.44 (m, 1H), 6.70-7.15 (m, 1H), 7.43 (t, J=7.5 Hz, 1H), 7.80-7.85 (m, 2H), 7.94 (d, J=8 Hz, 1H), 7.99-8.02 (m, 1H), 8.21 (d, J=8 Hz, 1H), 9.14 (s, 1H).

### Example 16

### Preparation of 1-(8-Bromo-6-chloro-12-methyl-12,13-dihydro-5H-11,12-diazabenzo[4,5]cyclohepta[1,2-b]naphthalen-5-yloxy)-3-imidazol-1-yl-propan-2-ol

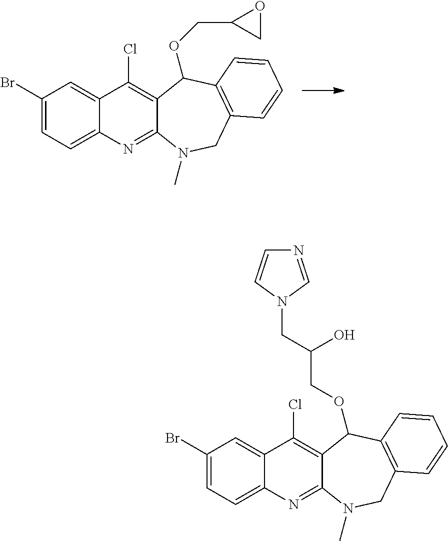

8-Bromo-6-chloro-12-methyl-5-oxiranylmethoxy-12,13-dihydro-5H-11,12-diaza-benzo[4,5]cyclohepta[1,2-b]naphthalene (0.4 g, 0.9 mmol), potassium carbonate (0.25 g, 1.8 mmol) and imidazole (0.24 g, 3.6 mmol) were refluxed in the presence of isopropanol (20 mL) for 12 h. Reaction mixture was concentrated under vacuum and extracted with ethyl acetate (50 mL×3). Organic layer was washed brine, dried over sodium sulphate and concentrated under vacuum to get crude mixture. Crude mixture was purified by column chromatography (silica gel 100-200 mesh, 5% methanol in dichloromethane), to get 1-(8-Bromo-6-chloro-12-methyl-12,13-dihydro-5H-11,12-diazabenzo[4,5]cyclohepta[1,2-b]naphthalen-5-yloxy)-3-imidazol-1-yl-propan-2-ol (0.21 g, 45%) as white solid. Mp 175-177° C. ¹H NMR (CDCl₃, 400 MHz): δ 2.87 (s, 3H), 3.15-3.28 (m, 1H), 3.30-3.42 (m, 1H, D₂O exchangeable), 3.44-3.58 (m, 1H), 3.78-4.44 (m, 4H), 5.39 (d, J=14 Hz, 1H), 5.80 (d, J=1.4 Hz, 1H), 6.70-7.15 (m, 2H), 7.28-7.50 (m, 5H), 7.68 (d, J=8.8 Hz, 1H), 7.74 (d, J=8.8 Hz, 1H), 8.20 (s, 1H).

The following compounds (general formulae I, II and III: Tables 2-4) were prepared as per the procedures described in the experimental section part one:

TABLE 2

I

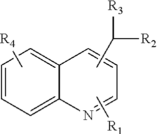

Description of the substituent variation in compounds prepared with the general formula I

| Serial No | R₁ | R₂ | R₃ | R₄ |
|---|---|---|---|---|
| 1 | 2-OMe | 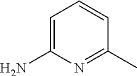 | Ph | 6-Br |
| 2 | 2-OMe | 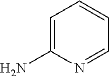 | Ph | 6-Br |
| 3 | 2-OMe | 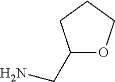 | Ph | 6-Br |
| 4 | 2-OMe | 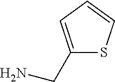 | Ph | 6-Br |
| 5a | 2-OMe | 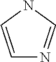 | Ph | 6-Br |
| 6a | 2-OMe | 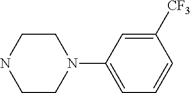 | Ph | 6-Br |
| 7a | 2-OMe | 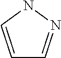 | Ph | 6-Br |
| 8a | 2-OMe | 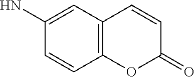 | Ph | 6-Br |
| 9 | 2-OMe | 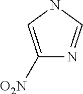 | Ph | 6-Br |
| 10 | 2-OMe | 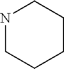 | Ph | 6-Br |
| 11 | 2-OMe | 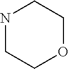 | Ph | 6-Br |
| 12 | 2-OMe | 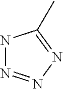 | Ph | 6-Br |
| 13 | 2-OMe | 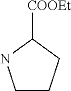 | Ph | 6-Br |
| 14 | 2-OMe | 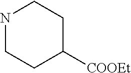 | Ph | 6-Br |
| 15 | 2-OMe | 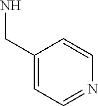 | Ph | 6-Br |
| 16 | 2-OMe | 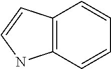 | Ph | 6-Br |
| 17 | 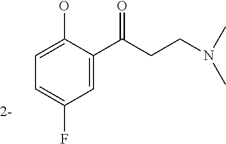 | H | Ph | 6-Br |
| 18 | 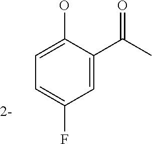 | H | Ph | 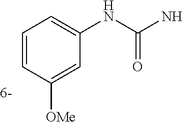 |
| 19 | 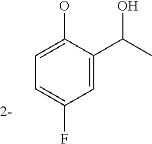 | H | Ph | 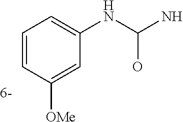 |
| 20 | 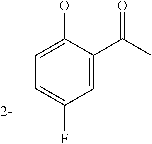 | H | Ph | 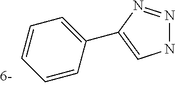 |
| 21 | 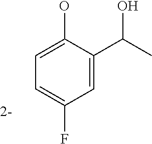 | H | Ph | 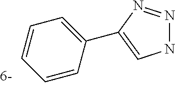 |
| 22 | 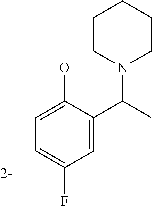 | H | Ph | 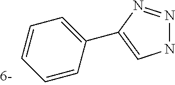 |
| 23a | 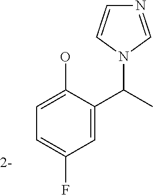 | H | Ph | 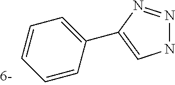 |
| 24 | 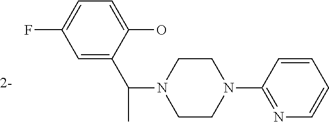 | H | Ph | 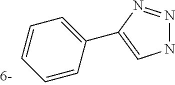 |
| 25 | 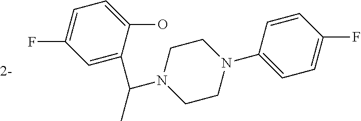 | H | Ph | 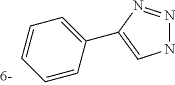 |
| 26a | 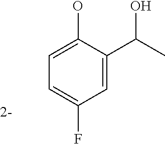 | H | Ph | 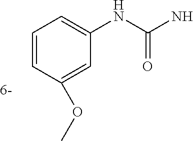 |
| 27 | 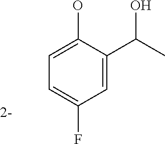 | H | Ph | 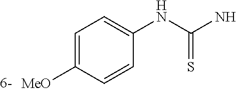 |
| 28 | 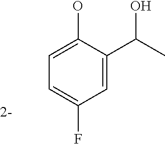 | H | Ph | 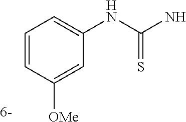 |
| 29 | 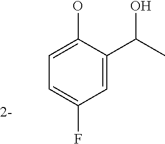 | H | Ph | 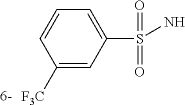 |
| 30 | 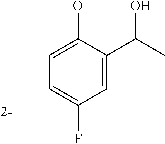 | H | Ph | 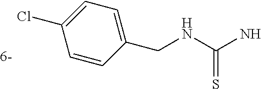 |
| 31a | 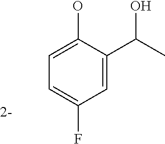 | H | Ph | 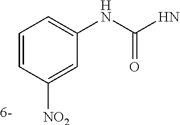 |
| 32 | 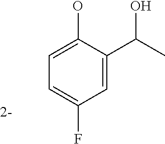 | H | Ph | 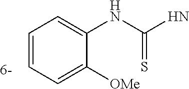 |
| 33 | 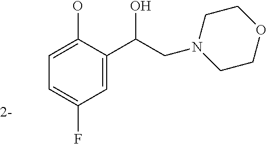 | H | Ph | 6-NO₂ |
| 34 | 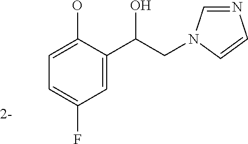 | H | Ph | 6-NO₂ |
| 35 | 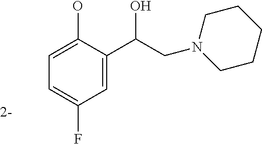 | H | Ph | 6-NO₂ |
| 36 | 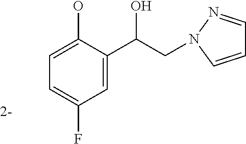 | H | Ph | 6-NO₂ |
| 37 | 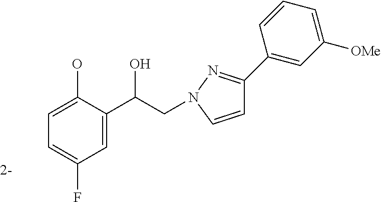 | H | Ph | 6-NO₂ |
| 38 | 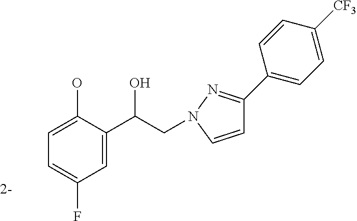 | H | Ph | 6-NO₂ |
| 39 | 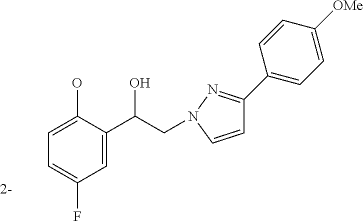 | H | Ph | 6-NO₂ |
| 40 | 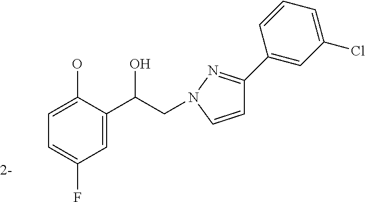 | H | Ph | 6-NO₂ |
| 41 | 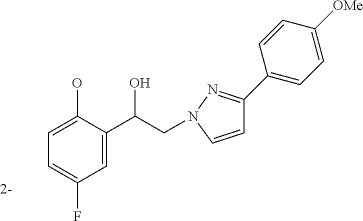 | H | Ph | 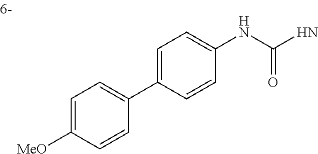 |
| 42* | 2-OMe | 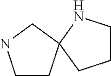 | Ph | 6-Br |
| 43* | 2-OMe | 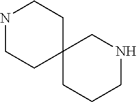 | Ph | 6-Br |
| 44* | 2-OMe | 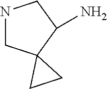 | Ph | 6-Br |
| 45* | 2-OMe | 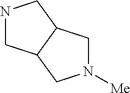 | Ph | 6-Br |
| 46* | 2-OMe | 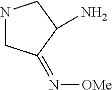 | Ph | 6-Br |
| 47* | 2-OMe | 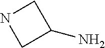 | Ph | 6-Br |
| 48* | 2-OMe | 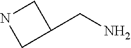 | Ph | 6-Br |
| 49* | 2-OMe | 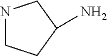 | Ph | 6-Br |
| 50* | 2-OMe | 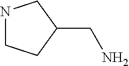 | Ph | 6-Br |
| 51* | 2-OMe | 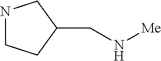 | Ph | 6-Br |
| 52* | 2-OMe | 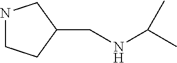 | Ph | 6-Br |
| 53* | 2-OMe | 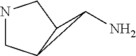 | Ph | 6-Br |
| 54* | 2-OMe | 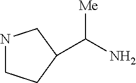 | Ph | 6-Br |
| 55* | 2-OMe | 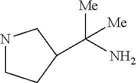 | Ph | 6-Br |
| 56* | 2-OMe | 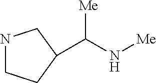 | Ph | 6-Br |
| 57* | 2-OMe | 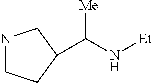 | Ph | 6-Br |
| 58* | 2-OMe | 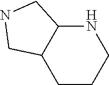 | Ph | 6-Br |
| 59* | 2-OMe | 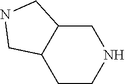 | Ph | 6-Br |
| 60* | 2-OMe | 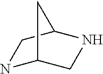 | Ph | 6-Br |
| 61* | 2-OMe | 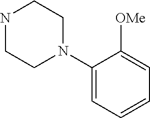 | Ph | 6-Br |
| 62* | 2-OMe | 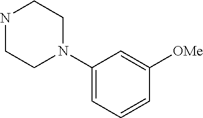 | Ph | 6-Br |
| 63* | 2-OMe | 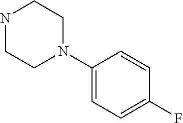 | Ph | 6-Br |
| 64* | 2-OMe | 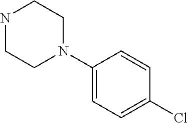 | Ph | 6-Br |
| 65* | 2-OMe | 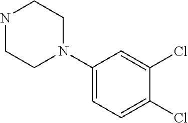 | Ph | 6-Br |
| 66* | 2-OMe | 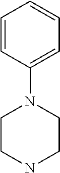 | Ph | 6-Br |
| 67* | 2-OMe | 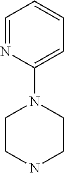 | Ph | 6-Br |
| 68* | 2-OMe | 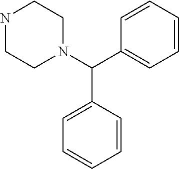 | Ph | 6-Br |
| 69* | 2-OMe | 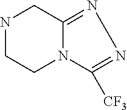 | Ph | 6-Br |
| 70* | 2-OMe | 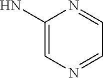 | Ph | 6-Br |
| 71* | 2-OMe | 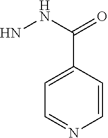 | Ph | 6-Br |

TABLE 3

II

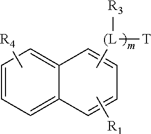

Description of the substituent variation in compounds prepared with the general formula
II

| Serial No | R₁ | R₃ | R₄ | T | L | m |
|---|---|---|---|---|---|---|
| 72a | H | Ph | H | 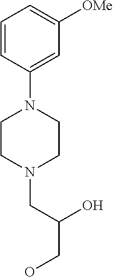 | CH | 1 |
| 73a | H | Ph | H | 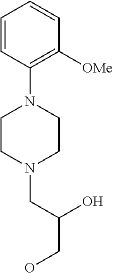 | CH | 1 |
| 74 | H | Ph | H | 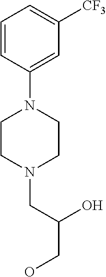 | CH | 1 |
| 75 | H | Ph | H | 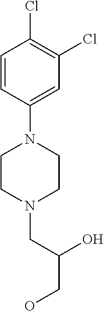 | CH | 1 |
| 76a | H | Ph | H | 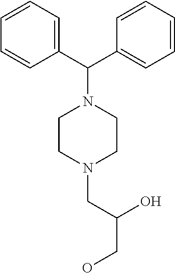 | CH | 1 |
| 77a | H | Ph | H | 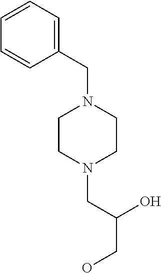 | CH | 1 |
| 78 | H | Ph | H | 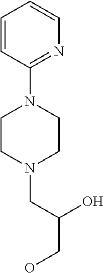 | CH | 1 |
| 79 | H | Ph | H | 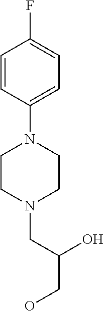 | CH | 1 |
| 80 | H | Ph | H | 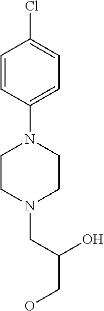 | CH | 1 |
| 81* | H | Ph | H | 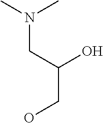 | CH | 1 |
| 82* | H | Ph | H | 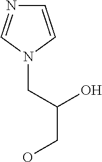 | CH | 1 |
| 83* | H | Ph | H | 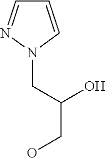 | CH | 1 |
| 84* | H | Ph | H | 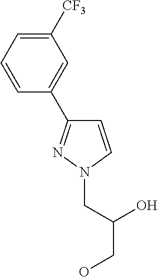 | H | 1 |

TABLE 4

III

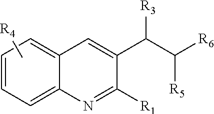

Description of the substituent variation in compounds prepared with the general formula
III

| Serial | | | | | | |
|---|---|---|---|---|---|---|
| No | R₁ | R₃ | R₄ | R₅ | R₆ | W |
| 85 | OMe | Ph | 6-NO₂ | 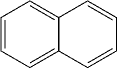 | 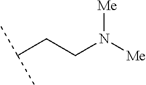 | COOH |
| 86 | OMe | Ph | 6-NO₂ | 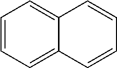 | — | COOH |
| 87 | OMe | Ph | 6-NO₂ | 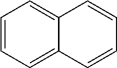 | 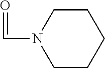 | — |
| 88 | OMe | Ph | 6-Br | 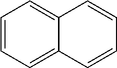 | COOEt | — |
| 89 | OMe | Ph | 6-Br | 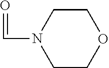 | COOMe | — |
| 90 | OMe | Ph | 6-Br | COOMe | 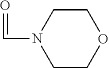 | — |
| 91 | OMe | Ph | 6-Br | 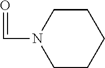 | COOMe | — |
| 92 | OMe | Ph | 6-Br | 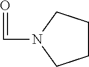 | COOMe | — |
| 93 | OMe | Ph | 6-Br | COOMe | 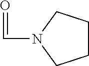 | — |
| 94 | OMe | Ph | 6-Br | 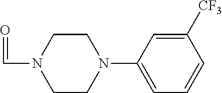 | COOMe | — |
| 95 | OMe | Ph | 6-Br | COOMe | 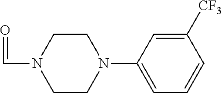 | — |
| 96 | OMe | Ph | 6-Br | 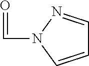 | COOMe | — |
| 97 | OMe | Ph | 6-Br | COOMe | 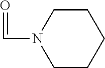 | — |
| 98* | OMe | Ph | 6-Br | 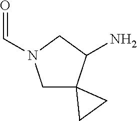 | COOMe | — |
| 99* | OMe | Ph | 6-Br | 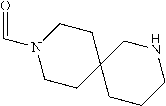 | COOMe | — |
| 100* | OMe | Ph | 6-Br | 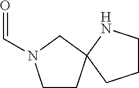 | COOMe | — |

Compounds marked with “a” have shown 99% inhibition at <4 μg/ml and described in Table 5.

### Conformationally Constrained Quinoline Compounds Prepared as Per the Description Given in Experimental Part Two

Different types of conformationally constrained compounds are disclosed in this document. G group is consisting of various subgroups (G₁ to G₆), which are expressed in Tables 1 and 5A-N.

TABLE 5
(Description of the substituent variation in compounds prepared with the general formula IV and V) 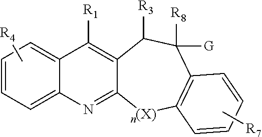
IV 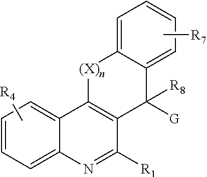
V
Subgroup G1:
R₈ ≠ H;
G = N~O—R₁₃ for the representative structures 52 and 135.

TABLE 5A
52

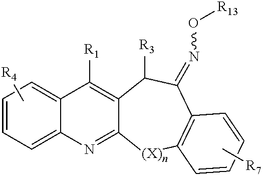

Description of the substituent variation in compounds prepared with the general formula 52

| Serial No. | X | n | R₁ | R₃ | R₄ | R₇ | R₁₃ |
|---|---|---|---|---|---|---|---|
| 101 | O | — | H | Ph | 9-Br | H | H |
| 102 | O | — | H | Ph | 9-Br | H | 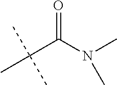 |
| 103 | O | — | H | Ph | 9-Br | H | 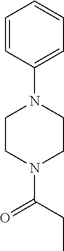 |
| 104 | O | — | H | Ph | 9-Br | F | 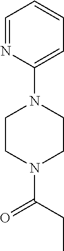 |
| 105 | O | — | H | Ph | 9-NO₂ | Cl | 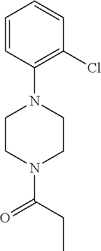 |
| 106 | O | — | H | Ph | 9-NH₂ | OCF₃ | 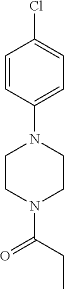 |
| 107 | O | — | H | Ph | 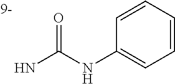 | Cl | 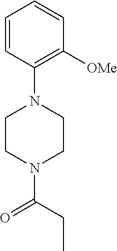 |
| 108 | O | — | H | Ph | 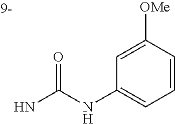 | Br | 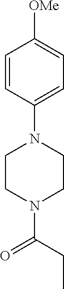 |
| 109 | O | — | H | Ph | 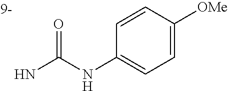 | F | 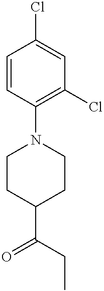 |
| 110 | O | — | H | Ph | 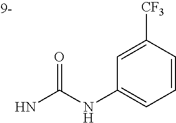 | CN | 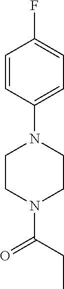 |
| 111 | O | — | H | Ph | 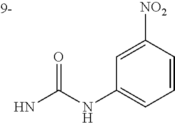 | OH | 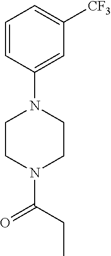 |
| 112 | O | — | H | Ph | 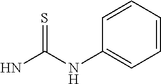 | NO₂ | 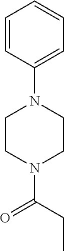 |
| 113 | O | — | H | Ph | 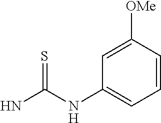 | F | 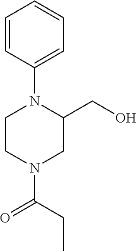 |
| 114 | O | — | H | Ph | 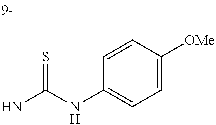 | F | 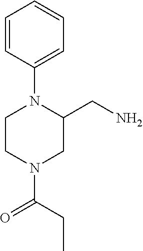 |
| 115 | O | — | H | Ph | 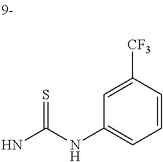 | CN | 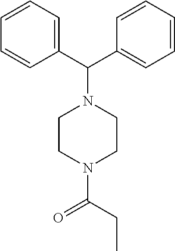 |
| 116 | O | — | H | Ph | 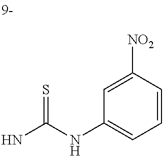 | F | 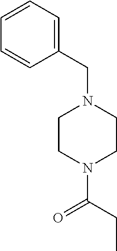 |
| 117 | O | — | H | Ph | 9-Br | F | 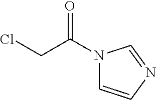 |
| 118 | O | — | H | Ph | 9-Br | CN | 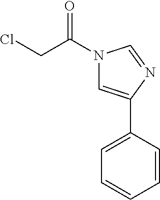 |
| 119 | O | — | H | Ph | 9-Br | NO₂ | 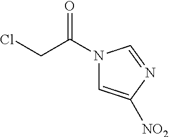 |
| 120 | O | — | H | Ph | 9-Br | F | 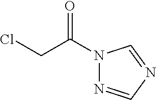 |
| 121 | O | — | H | Ph | 9-Br | Cl | 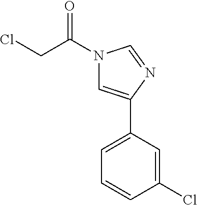 |
| 122 | O | — | H | Ph | 9-Br | OH | 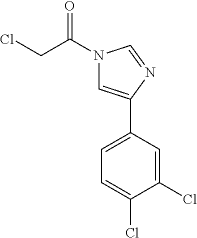 |
| 123 | O | — | H | Ph | 9-Br | OMe | 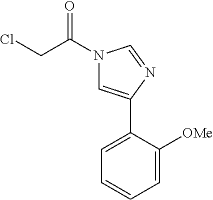 |
| 124 | O | — | H | Ph | 9-Br | F | 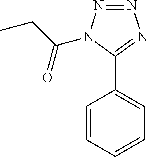 |
| 125 | O | — | H | Ph | 9-NO₂ | F | 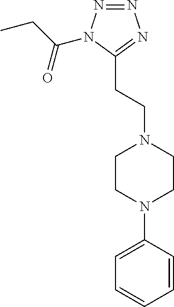 |

TABLE 5B
135

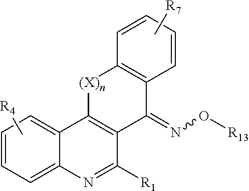

Description of the substituent variation in compounds prepared with the general formula 135

| Serial No. | X | n | R₁ | R₄ | R₇ | R₁₃ |
|---|---|---|---|---|---|---|
| 126 | CH₂ | 0 | OCH₃ | 2-Br | H | H |
| 127 | CH₂ | 0 | 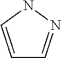 | 2-Br | H | H |
| 128 | CH₂ | 0 | OCH₃ | 2-Br | H | 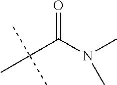 |
| 129a | CH₂ | 0 | 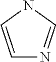 | 2-Br | H | H |
| 130 | CH₂ | 0 | OCH₃ | 2-Br | H | 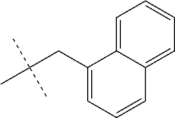 |
| 131 | CH₂ | 0 | | 2-Br | H | H |
| 132 | CH₂ | 0 | 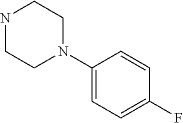 | 2-Br | H | 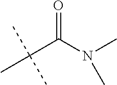 |
| 133 | CH₂ | 0 | 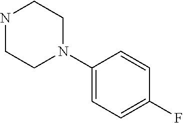 | 2-Br | H | 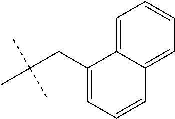 |
| 134a | CH₂ | 0 | 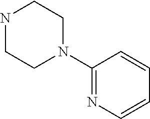 | 2-Br | H | H |
| 135a | CH₂ | 0 | 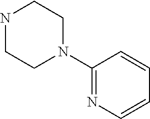 | 2-Br | H | 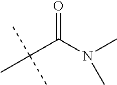 |
| 136 | CH₂ | 0 | 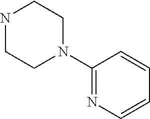 | 2-Br | H | 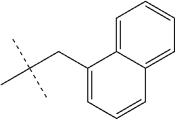 |
| 137 | CH₂ | 0 | 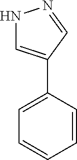 | 2-NO₂ | H | 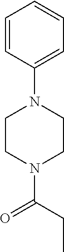 |
| 138 | CH₂ | 0 | 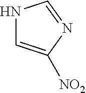 | 2-NH₂ | F | 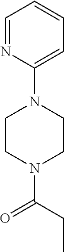 |
| 139 | CH₂ | 0 | 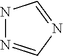 | 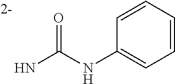 | Cl | 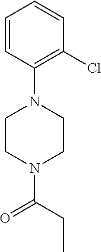 |
| 140 | CH₂ | 0 | 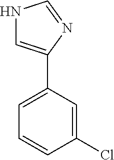 | 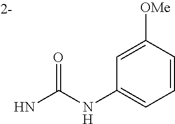 | OCF₃ | 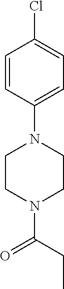 |
| 141 | CH₂ | 0 | 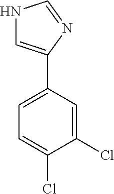 | 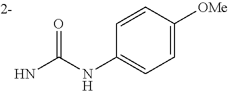 | Cl | 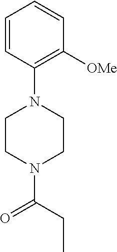 |
| 142 | CH₂ | 0 | 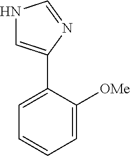 | 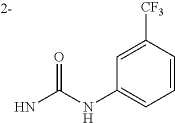 | Br | 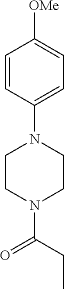 |
| 143 | CH₂ | 0 | 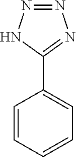 | 2-Br | F | 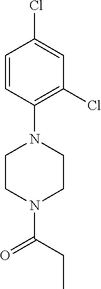 |
| 144 | CH₂ | 0 | 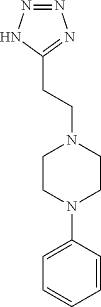 | 2-Br | CN | 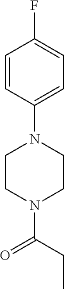 |
| 145 | CH₂ | 0 | 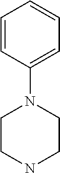 | 2-Br | OH | 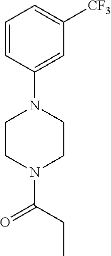 |
| 146 | CH₂ | 0 | 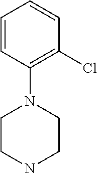 | 2-Br | NO₂ | 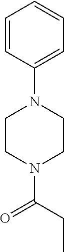 |
| 147 | CH₂ | 0 | 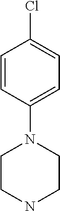 | 2-Br | F | 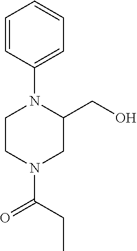 |
| 148 | CH₂ | 0 | 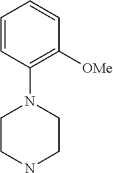 | 2-Br | F | 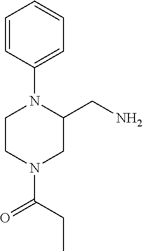 |
| 149 | CH₂ | 0 | 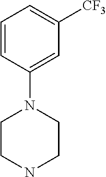 | 2-Br | CN | 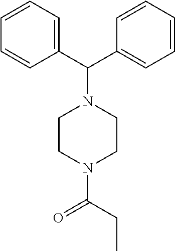 |
| 150 | CH₂ | 0 | 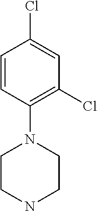 | 2-Br | F | 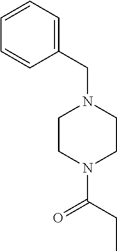 |

Subgroup G2:
R₈ = H, G = R₂ for the representative structures 136 and 137.

TABLE 5C
136

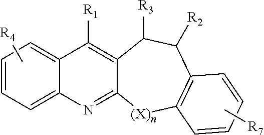

Description of the substituent variation in compounds prepared with the general formula 136

| Serial No. | X | n | R₁ | R₂ | R₃ | R₄ | R₇ |
|---|---|---|---|---|---|---|---|
| 151 | O | — | H | 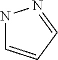 | Ph | 9-Br | H |
| 152 | O | — | H | 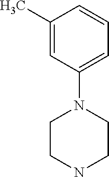 | Ph | 9-NO₂ | H |
| 153 | O | — | H | 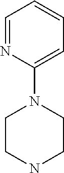 | Ph | 9-Br | H |
| 154 | O | — | H | 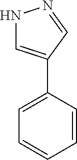 | Ph | 9-Br | F |
| 155 | O | — | H | 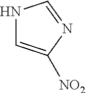 | Ph | 9-NO₂ | F |
| 156 | O | — | H | 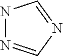 | Ph | 9-Br | CN |
| 157 | O | — | H | 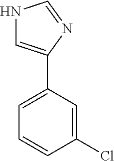 | Ph | 9-Br | OH |
| 158 | O | — | H | 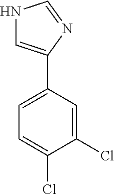 | Ph | 9-NO₂ | Cl |
| 159 | O | — | H | 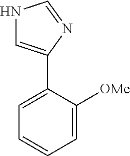 | Ph | 9-Br | Br |
| 160 | O | — | H | 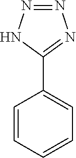 | Ph | 9-Br | NO₂ |
| 161 | O | — | H | 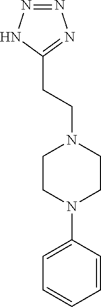 | Ph | 9-NO₂ | H |
| 162 | O | — | H | 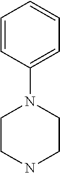 | Ph | 9-Br | H |
| 163 | O | — | H | 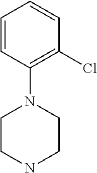 | Ph | 9-Br | F |
| 164 | O | — | H | 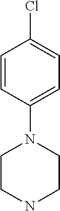 | Ph | 9-NO₂ | F |
| 165 | O | — | H | 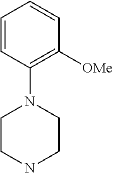 | Ph | 9-Br | H |
| 166 | O | — | H | 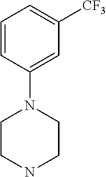 | Ph | 9-NO₂ | F |
| 167 | O | — | H | 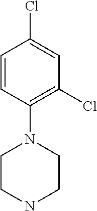 | Ph | 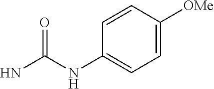 | H |

TABLE 5D
137

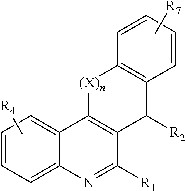

Description of the substituent variation in compounds prepared with the general formula 137.

| Serial No | X | n | R₁ | R₂ | R₄ | R₇ |
|---|---|---|---|---|---|---|
| 168 | CH₂ | 0 | OCH₃ | 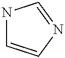 | 2-Br | H |
| 169 | CH₂ | 0 | 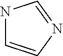 | 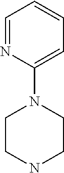 | 2-Br | H |
| 170 | CH₂ | 0 | OCH₃ | 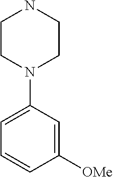 | 2-Br | H |
| 171 | CH₂ | 0 | 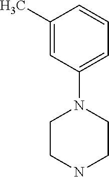 | 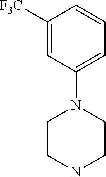 | 2-NH₂ | F |
| 172 | CH₂ | 0 | 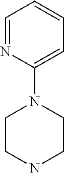 | 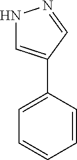 | 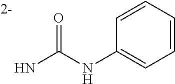 | CN |
| 173 | CH₂ | 0 | 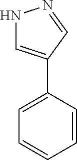 | 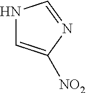 | 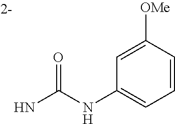 | OH |
| 174 | CH₂ | 0 | 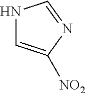 | 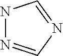 | 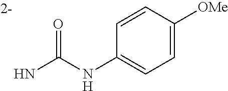 | Cl |
| 175 | CH₂ | 0 | 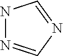 | 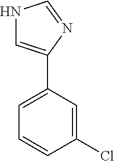 | 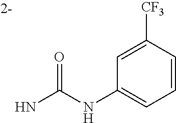 | Br |
| 176 | CH₂ | 0 | 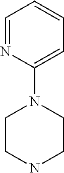 | 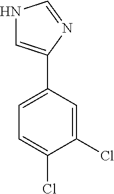 | 2-NH₂ | NO₂ |
| 177 | CH₂ | 0 | 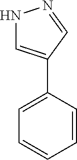 | 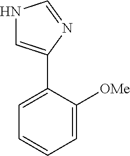 | 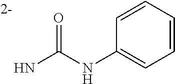 | F |
| 178 | CH₂ | 0 | 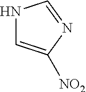 | 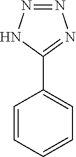 | 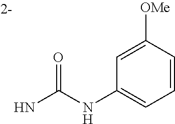 | CN |
| 179 | CH₂ | 0 | 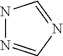 | 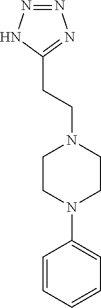 | 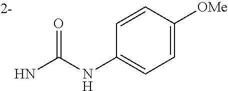 | OH |
| 180 | CH₂ | 0 | 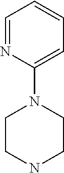 | 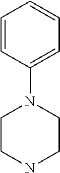 | 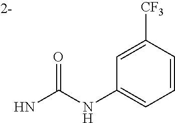 | Cl |
| 181 | CH₂ | 0 | 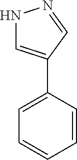 | 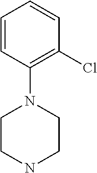 | 2-Br | Br |
| 182 | CH₂ | 0 | 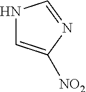 | 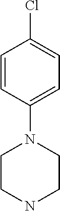 | 2-Br | NO₂ |
| 183 | CH₂ | 0 | 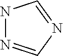 | 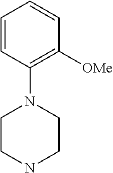 | 2-Br | F |
| 184 | CH₂ | 0 | 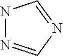 | 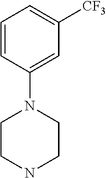 | 2-Br | F |
| 185 | CH₂ | 0 | 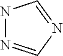 | 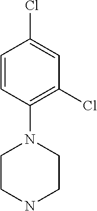 | 2-Br | F |

Subgroup G3:
R8 = H, G is represented by formula 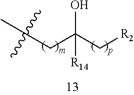
or 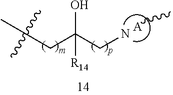
For the representative structurtes 138 nad 139

TABLE 5E
138

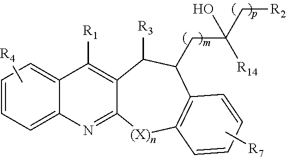

Description of the substituent variation in compounds prepared with the general formula 138

| Serial No | X | n | R₁ | R₂ | R₃ | R₄ | R₇ | m | p | R₁₄ |
|---|---|---|---|---|---|---|---|---|---|---|
| 186 | O | — | H | 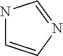 | Ph | 9-Br | H | 1 | 1 | 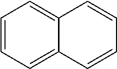 |
| 187 | O | — | H | 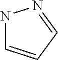 | Ph | 9-Br | H | 1 | 1 | 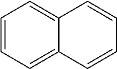 |
| 188 | O | — | H | 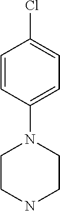 | Ph | 9-NO₂ | 3-F | 1 | 1 | 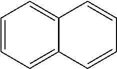 |
| 189 | O | — | H | OCH₃ | Ph | 9-NH₂ | H | 1 | 1 | 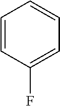 |
| 190 | O | — | H | 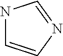 | Ph | 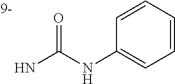 | H | 1 | 1 | 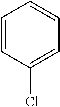 |
| 191 | O | — | H | OCH₃ | Ph | 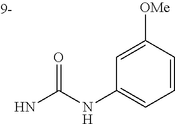 | 3-F | 1 | 1 | 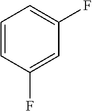 |
| 192 | O | — | H | 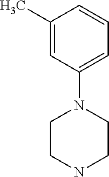 | Ph | 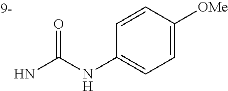 | H | 1 | 1 | 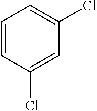 |
| 193 | O | — | H | 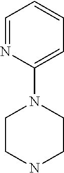 | Ph | 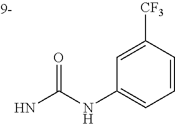 | H | 1 | 1 | 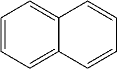 |
| 194 | O | — | H | 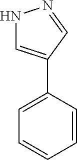 | Ph | 9-Br | 3-F | 1 | 1 | 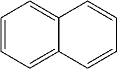 |
| 195 | O | — | H | 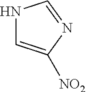 | Ph | 9-Br | H | 1 | 1 | 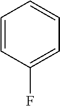 |
| 196 | O | — | H | 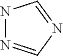 | Ph | 9-Br | H | 1 | 1 | 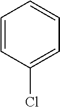 |
| 197 | O | — | H | 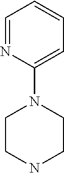 | Ph | 9-Br | 3-NO₂ | 1 | 1 | 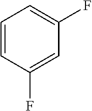 |
| 198 | O | — | H | 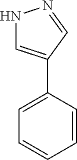 | Ph | 9-Br | 3-F | | | 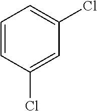 |

TABLE 5F
139

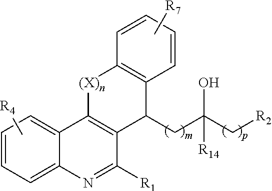

Description of the substituent variation in compounds prepared with the general formula 139

| Serial No | X | n | R₁ | R₂ | R₄ | R₇ | m | p | R₁₄ |
|---|---|---|---|---|---|---|---|---|---|
| 199 | CH₂ | 0 | OCH₃ | 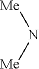 | 2-Br | H | 0 | 2 | 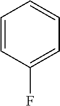 |
| 200 | CH₂ | 1 | OCH₃ | 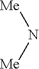 | 2-Br | H | 0 | 2 | 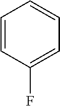 |
| 201 | CH₂ | 0 | OCH₃ | 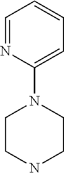 | 2-Br | H | 1 | 1 | 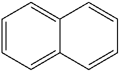 |
| 202 | CH₂ | 0 | OCH₃ | 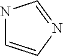 | 2-Br | H | 1 | 1 | 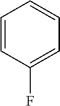 |
| 203 | CH₂ | 0 | OCH₃ | 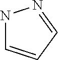 | 2-Br | H | 1 | 1 | 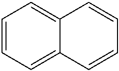 |
| 204 | CH₂ | 0 | 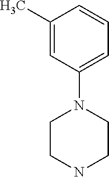 | 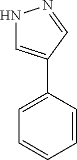 | 2-NO₂ | 3-F | 0 | 2 | 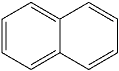 |
| 205 | CH₂ | 1 | 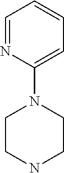 | 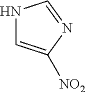 | 2-NH₂ | H | 0 | 2 | 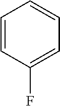 |
| 206 | CH₂ | 0 | 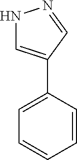 | 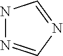 | 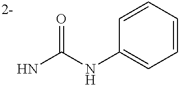 | H | 1 | 1 | 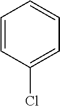 |
| 207 | CH₂ | 0 | 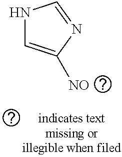 | 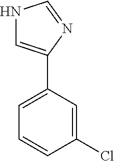 | 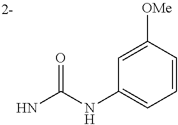 | 3-F | 1 | 1 | 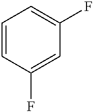 |
| 208 | CH₂ | 0 | 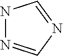 | 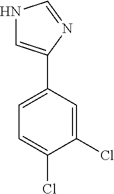 | 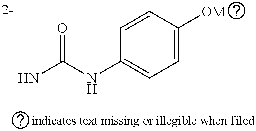 | H | 1 | 1 | 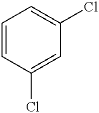 |
| 209 | CH₂ | 0 | 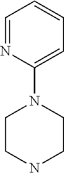 | 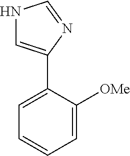 | 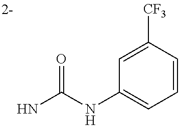 | H | 0 | 2 | 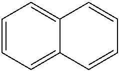 |
| 210 | CH₂ | 1 | 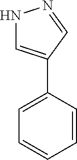 | 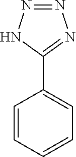 | 2-Br | 3-F | 0 | 2 | 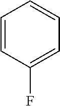 |
| 211 | CH₂ | 0 | 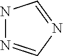 | 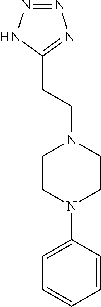 | 2-Br | H | 1 | 1 | 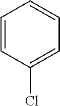 |
| 212 | CH₂ | 0 | 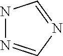 | 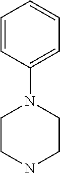 | 2-NO₂ | H | 1 | 1 | 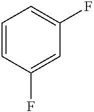 |
| 213 | CH₂ | 0 | 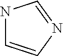 | 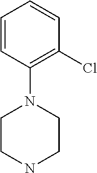 | 2-NH₂ | 3-NO₂ | 1 | 1 | 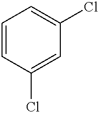 |
| 214 | CH₂ | 0 | 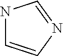 | 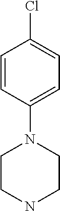 | 2-Br | 3-F | 0 | 2 | 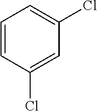 |
| 215 | CH₂ | 1 | 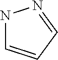 | 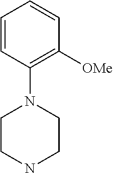 | 2-Br | 3-F | 0 | 2 | 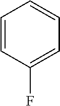 |
| 216 | CH₂ | 0 | 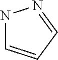 | 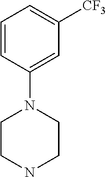 | 2-NO₂ | H | 1 | 1 | 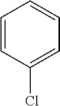 |
| 217 | CH₂ | 0 | 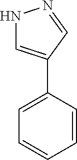 | 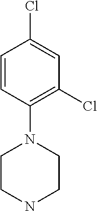 | 2-NH₂ | H | 1 | 1 | 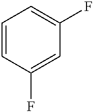 |

Subgroup G₄: R₈ = CH₃, G = YH or represented by formula 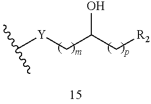
or 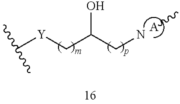
For the representative structurtes 140 and 141

TABLE 5G
140

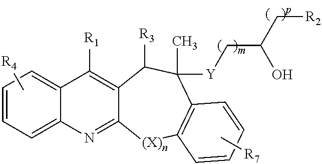

Description of the substituent variation in compounds prepared with the general formula 140

| Serial | | | | | | | | | | |
|---|---|---|---|---|---|---|---|---|---|---|
| No. | X | n | R₁ | R₂ | R₃ | R₄ | R₇ | Y | m | p |
| 218 | O | — | H | — | Ph | 9-Br | 3-F | O | — | — |
| 219 | O | — | H | — | Ph | 9-Br | H | O | — | — |
| 220 | O | — | H | — | Ph | 9-Br | H | O | — | — |
| 221 | O | — | H | 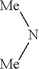 | | 9-NO₂ | H | O | 1 | 1 |
| 222 | O | — | H | 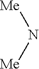 | Ph | 9-NH2 | H | O | 1 | 1 |
| 223 | O | — | H | 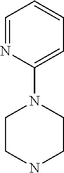 | Ph | 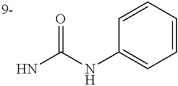 | 3-F | O | 1 | 1 |
| 224 | O | — | H | 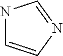 | Ph | 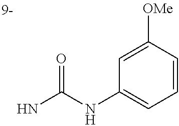 | H | O | 1 | 1 |
| 225 | O | — | H | 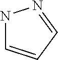 | Ph | 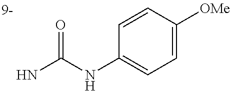 | H | O | 1 | 1 |
| 226 | O | — | H | 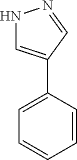 | Ph | 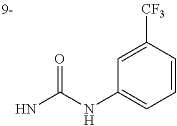 | 3-F | O | 1 | 1 |
| 227 | O | — | H | 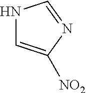 | Ph | 9-Br | H | O | 1 | 1 |
| 228 | O | — | H | 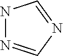 | Ph | 9-Br | H | O | 1 | 1 |
| 229 | O | — | H | 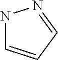 | Ph | 9-Br | 3-F | O | 1 | 1 |
| 230 | O | — | H | 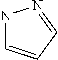 | Ph | 9-Br | H | O | 1 | 1 |
| 231 | O | — | H | 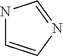 | Ph | 9-Br | H | O | 1 | 1 |
| 232 | O | — | H | 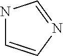 | Ph | 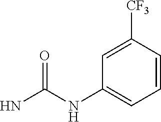 | 3-NO₂ | O | 1 | |

TABLE 5H
141

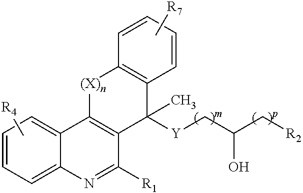

Description of the substituent variation in compounds prepared with the general formula 141

| Serial No | X | n | R₁ | R₂ | R₄ | R₇ | Y | m | p |
|---|---|---|---|---|---|---|---|---|---|
| 233 | CH₂ | 0 | 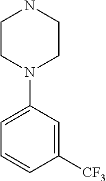 | — | 2-Br | H | OH | — | — |
| 234a | CH₂ | 0 | 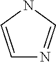 | — | 2-Br | H | OH | — | — |
| 235a | CH₂ | 0 | OCH₃ | 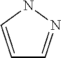 | 2-Br | H | O | 1 | 1 |
| 236a | CH₂ | 0 | OCH₃ | 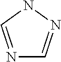 | 2-Br | H | O | 1 | 1 |
| 237 | CH₂ | 0 | OCH₃ | 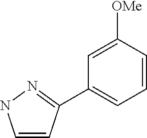 | 2-Br | H | O | 1 | 1 |
| 238a | CH₂ | 0 | OCH₃ | 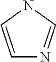 | 2-Br | H | O | 1 | 1 |
| 239 | CH₂ | 0 | OCH₃ | 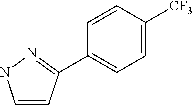 | 2-Br | H | O | 1 | 1 |
| 240 | CH₂ | 0 | OCH₃ | 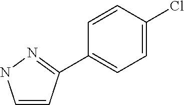 | 2-Br | H | O | 1 | 1 |
| 241a | CH₂ | 0 | OCH₃ | 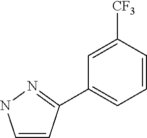 | 2-Br | H | O | 1 | 1 |
| 242a | CH₂ | 0 | OCH₃ | 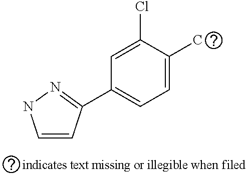 | 2-Br | H | O | 1 | 1 |
| 243 | CH₂ | 0 | OCH₃ | 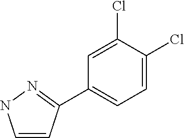 | 2-Br | H | O | 1 | 1 |
| 244 | CH₂ | 0 | OCH₃ | 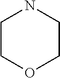 | 2-Br | H | O | 1 | 1 |
| 245 | CH₂ | 0 | OCH₃ | 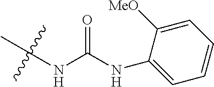 | 2-Br | H | O | 1 | 1 |
| 246 | CH₂ | 0 | OCH₃ | 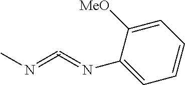 | 2-Br | H | O | 1 | 1 |

Subgroup G5: R₈ = OR₁₅, G = CH₃ or can be represented by formula 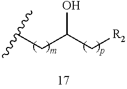
or 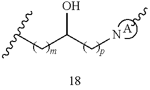
For the representative structurtes 142 and 143

TABLE 5I
142

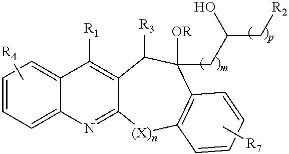

Description of the substituent variation in compounds prepared with the general formula 142

| Serial No | X | n | R₁ | R₂ | R₃ | R₄ | R₇ | R₁₅ | m | p |
|---|---|---|---|---|---|---|---|---|---|---|
| 247 | O | — | H | 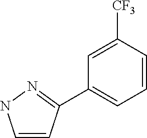 | Ph | 9-Br | H | CH₃ | 0 | 1 |
| 248 | O | — | H | 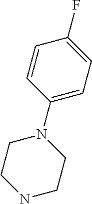 | Ph | 9-Br | H | CH₃ | 0 | 1 |
| 249 | O | — | H | 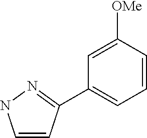 | Ph | 9-Br | 3-F | 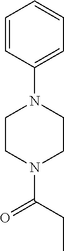 | 1 | 1 |
| 250 | O | — | H | 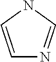 | Ph | 9-NO₂ | 3-F | 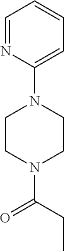 | 1 | 1 |
| 251 | O | — | H | 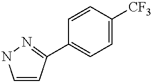 | Ph | 9-NH₂ | 3-CN | 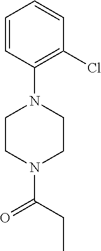 | 1 | 1 |
| 252 | O | — | H | 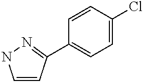 | Ph | 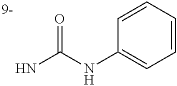 | 3-F | 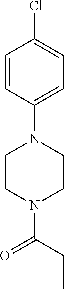 | 1 | 1 |
| 253 | O | — | H | 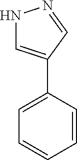 | Ph | 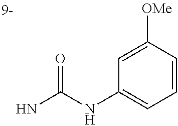 | 3-F | 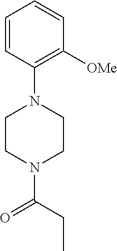 | 1 | 1 |
| 254 | O | — | H | 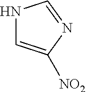 | Ph | 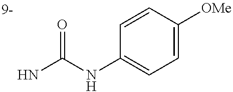 | H | 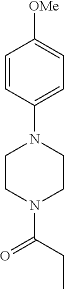 | 1 | 1 |
| 255 | O | — | H | 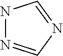 | Ph | 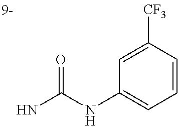 | H | 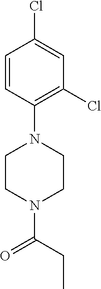 | 1 | 1 |
| 256 | O | — | H | 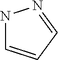 | Ph | 9-Br | H | 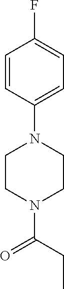 | 1 | 1 |
| 257 | O | — | H | 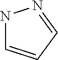 | Ph | 9-Br | 3-NO₂ | 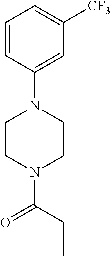 | 1 | 1 |
| 258 | O | — | H | 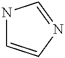 | Ph | 9-Br | 3-OCH₃ | 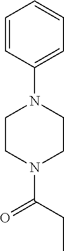 | 1 | 1 |
| 259 | O | — | H | 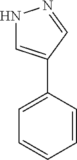 | Ph | 9-Br | 3-NO₂ | 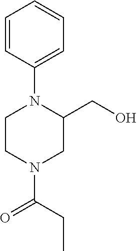 | 1 | 1 |
| 260 | O | — | H | 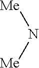 | Ph | 9-Br | 3-OCH₃ | 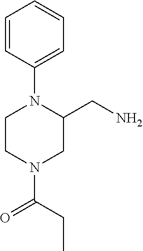 | 1 | 1 |
| 261 | O | — | H | 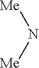 | Ph | 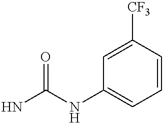 | H | 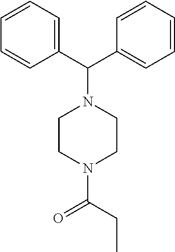 | 1 | 1 |
| 262 | O | — | H | 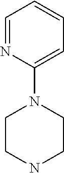 | Ph | 9-Br | H | 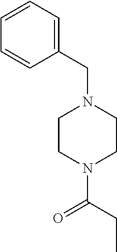 | 1 | 1 |

TABLE 5J
143

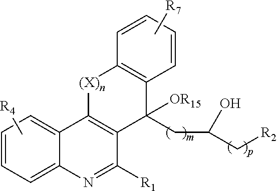

Description of the substituent variation in compounds prepared with the general formula 143

| Serial No | X | n | R₁ | R₂ | R₄ | R₇ | R₁₅ | m | p |
|---|---|---|---|---|---|---|---|---|---|
| 263a | CH₂ | 0 | OCH₃ | — | 2-Br | H | 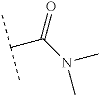 | — | — |
| 264 | CH₂ | 0 | OCH₃ | 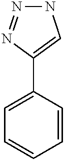 | 2-Br | H | CH₃ | 0 | 1 |
| 265 | CH₂ | 0 | OCH₃ | 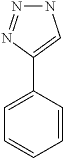 | 2-Br | H | CH₃ | 0 | 1 |
| 266 | CH₂ | 0 | 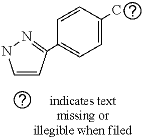 | 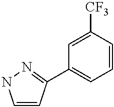 | 2-Br | 3-F | 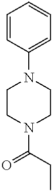 | 0 | 1 |
| 267 | CH₂ | 0 | 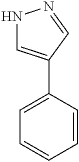 | 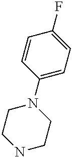 | 2-NO₂ | 3-F | 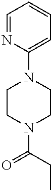 | 0 | 1 |
| 268 | CH₂ | 0 | 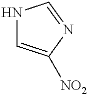 | 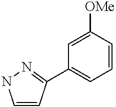 | 2-NH₂ | 3-CN | 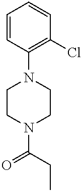 | 0 | 1 |
| 269 | CH₂ | 0 | 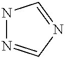 | 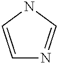 | 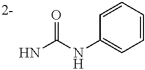 | 3-F | 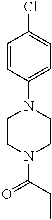 | 0 | 1 |
| 270 | CH₂ | 0 | 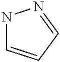 | 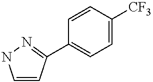 | 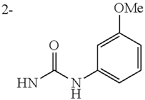 | 3-F | 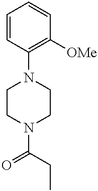 | 1 | 1 |
| 271 | CH₂ | 0 | 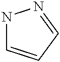 | 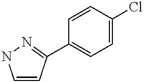 | 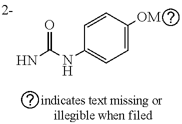 | H | 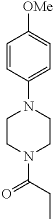 | 1 | 1 |
| 272 | CH₂ | 0 | 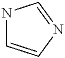 | 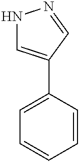 | 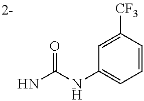 | H | 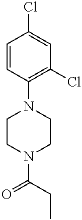 | 1 | 1 |
| 273 | CH₂ | 0 | 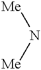 | 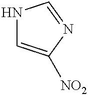 | 2-NO₂ | H | 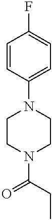 | 1 | 1 |
| 274 | CH₂ | 0 | 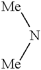 | 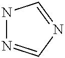 | 2-NH₂ | 3-NO₂ | 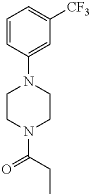 | 1 | 1 |
| 275 | CH₂ | 0 | 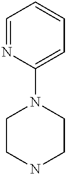 | 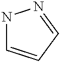 | 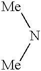 | 3- OCH₃ | 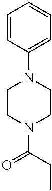 | 1 | 1 |
| 276 | CH₂ | 0 | 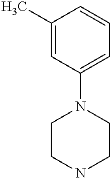 | 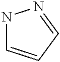 | 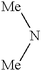 | 3-NO₂ | 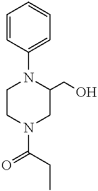 | 1 | 1 |
| 277 | CH₂ | 0 | 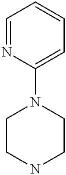 | 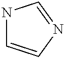 | 2-Br | 3- OCH₃ | 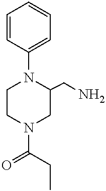 | 1 | 1 |
| 278 | CH₂ | 0 | OCH₃ | 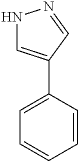 | 2-Br | H | 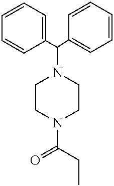 | 1 | 1 |
| 279 | CH₂ | 0 | OCH₃ | 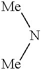 | 2-Br | H | 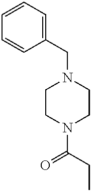 | 1 | 1 |
| 280 | CH₂ | 0 | OCH₃ | 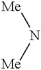 | 2-Br | 3-Cl | CH₃ | 1 | 1 |

Subgroup G₆: R₈ = 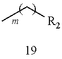
or 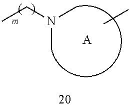
Then G is expressed with formula 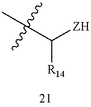
or 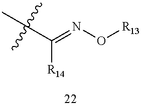
For the representative structurtes 144-147

TABLE 5K
144

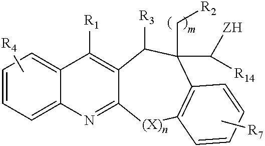

Description of the substituent variation in compounds prepared with the general formula 144

| Serial No | X | n | R₁ | R₂ | R₃ | R₄ | R₇ | Z | R₁₄ | m |
|---|---|---|---|---|---|---|---|---|---|---|
| 281 | O | — | H | 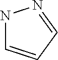 | Ph | 9-Br | H | O | 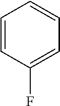 | 2 |
| 282 | O | — | H | 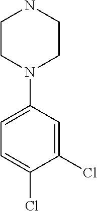 | Ph | 9-Br | H | O | 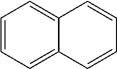 | 2 |
| 283 | O | — | H | 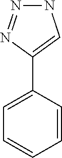 | Ph | 9-NO₂ | 4-F | O | 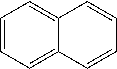 | 2 |
| 284 | O | — | H | 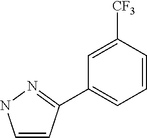 | Ph | 9-NH₂ | 4-F | O | 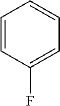 | 2 |
| 285 | O | — | H | 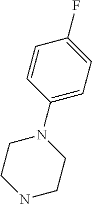 | Ph | 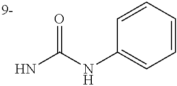 | 4-F | O | 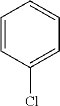 | 2 |
| 286 | O | — | H | 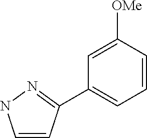 | Ph | 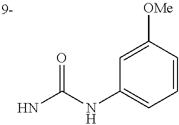 | 4-F | O | 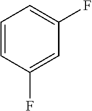 | 2 |
| 287 | O | — | H | 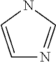 | Ph | 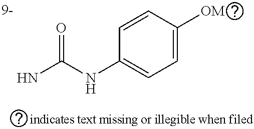 | 4- OCH3 | O | 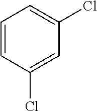 | 2 |
| 288 | O | — | H | 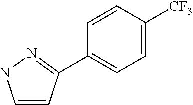 | Ph | 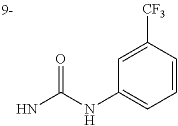 | 4- OCH3 | O | 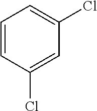 | 2 |
| 289 | O | — | H | 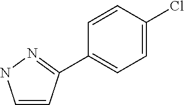 | Ph | 9-Br | 4- OCH₃ | O | 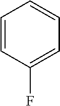 | 2 |
| 290 | O | — | H | 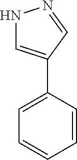 | Ph | 9-Br | H | O | 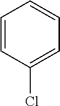 | 2 |
| 291 | O | — | H | 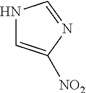 | Ph | 9-Br | H | O | 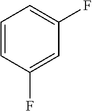 | 2 |
| 292 | O | — | H | 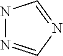 | Ph | 9-Br | 3-NO₂ | O | 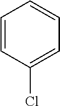 | 2 |

TABLE 5L
145

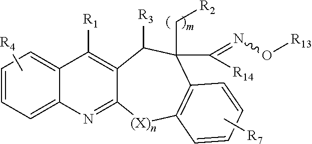

Description of the substituent variation in compounds prepared with the general formula 145

| Serial No | X | n | R₁ | R₂ | R₃ | R₄ | R₇ | R₁₃ | R₁₄ | m |
|---|---|---|---|---|---|---|---|---|---|---|
| 293 | O | — | H | 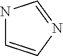 | Ph | 9-Br | H | H | 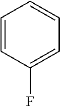 | 2 |
| 294 | O | — | H | 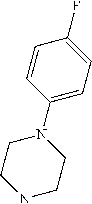 | Ph | 9-NO₂ | 4-F | CH₃ | 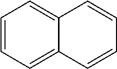 | 2 |
| 295 | O | — | H | 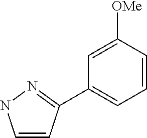 | Ph | 9-NH₂ | 4-F | 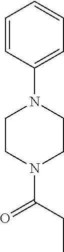 | 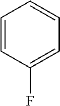 | 2 |
| 296 | O | — | H | 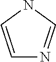 | Ph | 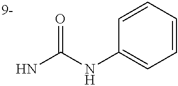 | 4-F | 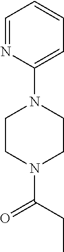 | 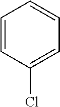 | 2 |
| 297 | O | — | H | 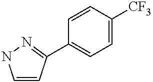 | Ph | 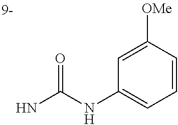 | 4-OCH₃ | 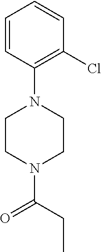 | 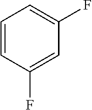 | 2 |
| 298 | O | — | H | 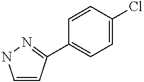 | Ph | 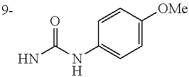 | 4-OCH₃ | 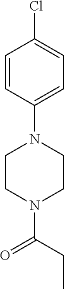 | 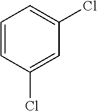 | 2 |
| 299 | O | — | H | 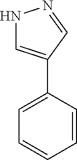 | Ph | 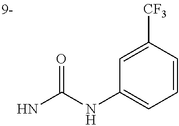 | 4-OCH₃ | 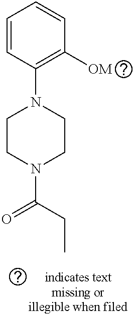 | 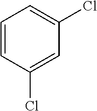 | 2 |
| 300 | O | — | H | 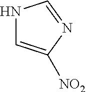 | Ph | 9-Br | H | 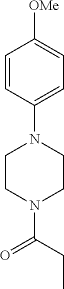 | 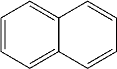 | 2 |
| 301 | O | — | H | 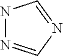 | Ph | 9-Br | H | 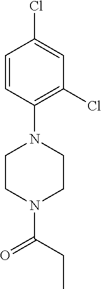 | 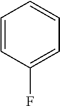 | 2 |
| 302 | O | — | H | 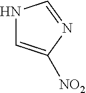 | Ph | 9-Br | 3-NO₂ | 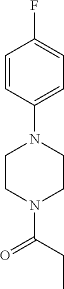 | 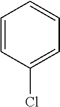 | 2 |

TABLE 5M
146

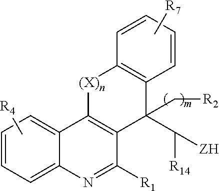

Description of the substituent variation in compounds prepared with the general formula 146

| Serial | | | | | | | | | |
|---|---|---|---|---|---|---|---|---|---|
| No | X | n | R₁ | R₂ | R₄ | R₇ | Z | R₁₄ | m |
| 303 | CH₂ | 0 | OCH₃ | 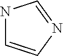 | 2-Br | H | O | 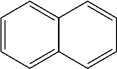 | 2 |
| 304 | CH₂ | 0 | OCH₃ | 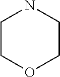 | 2-Br | H | O | 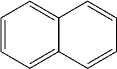 | 2 |
| 305 | CH₂ | 0 | 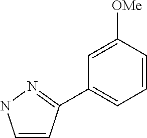 | 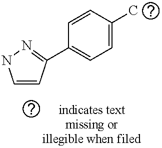 | 2-NO₂ | H | O | 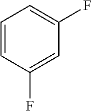 | 2 |
| 306 | CH₂ | 0 | 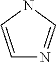 | 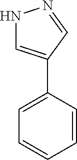 | 2-NH₂ | 4-F | O | 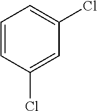 | 2 |
| 307 | CH₂ | 0 | 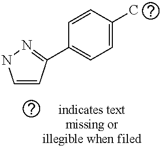 | 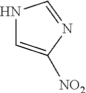 | 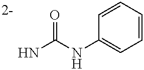 | 4-F | O | 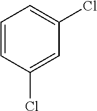 | 2 |
| 308 | CH₂ | 0 | 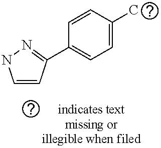 | 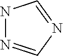 | 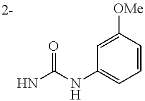 | 4-F | O | 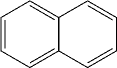 | 2 |
| 309 | CH₂ | 0 | 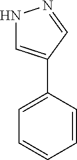 | 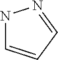 | 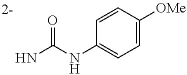 | 4-OCH₃ | O | 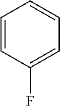 | 2 |
| 310 | CH₂ | 0 | 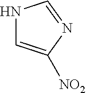 | 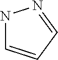 | 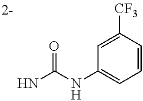 | 4-OCH₃ | O | 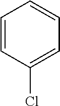 | 2 |
| 311 | CH₂ | 0 | 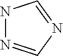 | 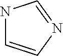 | 2-Br | 4-OCH₃ | O | 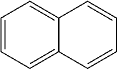 | 2 |
| 312 | CH₂ | 0 | 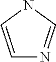 | 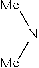 | 2-Br | H | O | 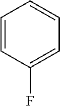 | 2 |
| 313 | CH₂ | 0 | 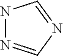 | 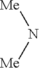 | 2-NH₂ | H | O | 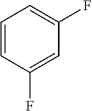 | 2 |
| 314 | CH₂ | 0 | 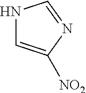 | 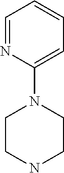 | 2-NO₂ | H | O | 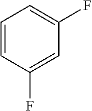 | 2 |

TABLE 5N
147

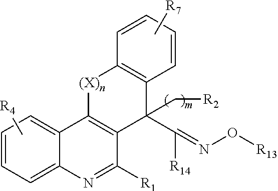

Description of the substituent variation in compounds prepared with the general formula 147

| Serial | | | | | | | | | |
|---|---|---|---|---|---|---|---|---|---|
| No | X | n | R₁ | R₂ | R₄ | R₇ | R₁₃ | R₁₄ | m |
| 315 | CH₂ | 0 | OCH₃ | 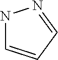 | 2-Br | H | H | 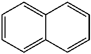 | 2 |
| 316 | CH₂ | 0 | OCH₃ | 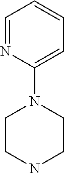 | 2-Br | H | H | 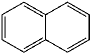 | 2 |
| 317 | CH₂ | 0 | 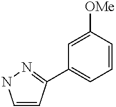 | 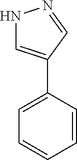 | 2-NO₂ | H | CH₃ | 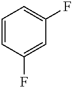 | 2 |
| 318 | CH₂ | 0 | 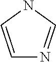 | 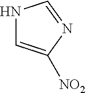 | 2-NH₂ | H | 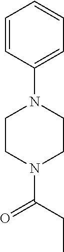 | 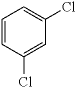 | 2 |
| 319 | CH₂ | 0 | 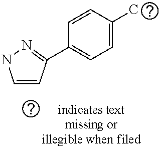 | 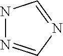 | 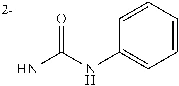 | 4-F | 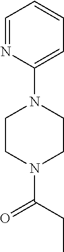 | 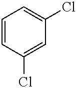 | 2 |
| 320 | CH₂ | 0 | 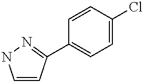 | 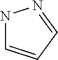 | 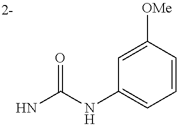 | 4-F | 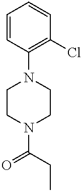 | 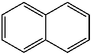 | 2 |
| 321 | CH₂ | 0 | 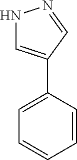 | 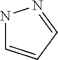 | 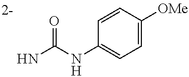 | 4-F | 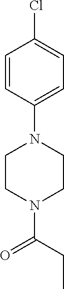 | 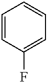 | 2 |
| 322 | CH₂ | 0 | 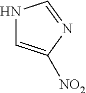 | 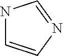 | 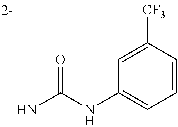 | 4- OCH₃ | 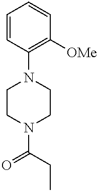 | 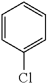 | 2 |
| 323 | CH₂ | 0 | 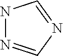 | 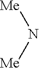 | 2-Br | 4- OCH₃ | 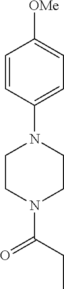 | 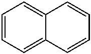 | 2 |
| 324 | CH₂ | 0 | 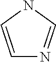 | 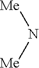 | 2-Br | 4- OCH₃ | 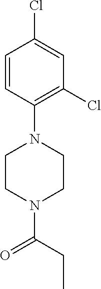 | 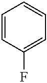 | 2 |
| 325 | CH₂ | 0 | 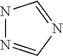 | 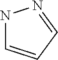 | 2-Br | H | 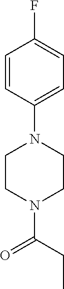 | 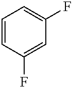 | 2 |

TABLE 6
Description of the substituent variation in compounds
prepared with the general formula VI
VI

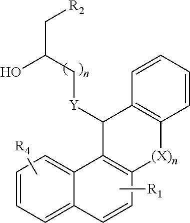

| Serial | | | | | | |
|---|---|---|---|---|---|---|
| No | X | N | Y | R₁ | R₂ | R₄ |
| 326 | CH₂ | 1 | O | H | 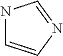 | 2-Br |
| 327 | CH₂ | 1 | O | 6-OCH₃ | 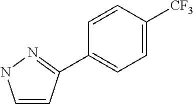 | 2-Br |
| 328 | CH₂ | 1 | O | 6-OCH₃ | 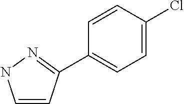 | 2-Br |
| 329 | CH₂ | 1 | O | 6-OCH₃ | 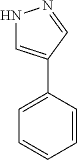 | 2-Br |
| 330 | CH₂ | 1 | O | 6-Br | 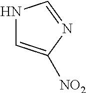 | 2- OH |
| 331 | CH₂ | 1 | O | 6-Br | 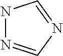 | 2- NO₂ |
| 332 | CH₂ | 1 | O | 6-Br | 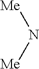 | 2- NH₂ |
| 333 | CH₂ | 1 | O | 6-Br | 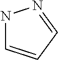 | 2-Br |
| 334 | CH₂ | 1 | O | 6-Br | 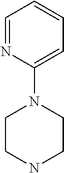 | 2-Br |
| 335 | CH₂ | 1 | O | 6-Br | 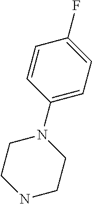 | 2-Br |

TABLE 7
Description of the substituent variation in compounds prepared
with the general formula VII
VII

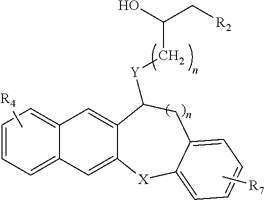

| Serial | | | | | | |
|---|---|---|---|---|---|---|
| No | X | n | Y | R₂ | R₄ | R₇ |
| 336 | CH₂ | 1 | O | 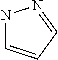 | H | 3-F |
| 337 | CH₂ | 1 | O | 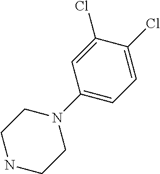 | H | H |
| 338 | CH₂ | 1 | O | 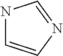 | 9-Br | H |
| 339 | CH₂ | 1 | O | 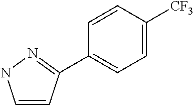 | 9-Br | 3-F |
| 340 | CH₂ | 1 | O | 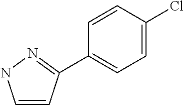 | 9-Br | 3-F |
| 341 | CH₂ | 1 | O | 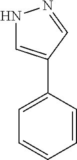 | 9-Br | 3-F |
| 342 | CH₂ | 1 | O | 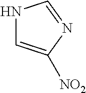 | 9-OH | 3-OCH₃ |
| 343 | CH₂ | 1 | O | 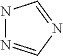 | 9-NO₂ | 3-OCH₃ |
| 344 | CH₂ | 1 | O | 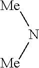 | 9- NH₂ | 3-OCH₃ |
| 345 | CH₂ | 1 | O | 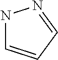 | 9-Br | H |
| 346 | CH₂ | 1 | O | 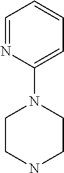 | 9-Br | 3-F |
| 347 | CH₂ | 1 | O | 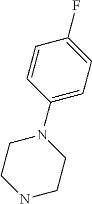 | 9-OH | 3-F |

TABLE 8
Description of the substituent variation in compounds
prepared with the general formula VIII
VIII

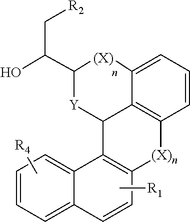

| Serial | | | | | | |
|---|---|---|---|---|---|---|
| No | X | n | Y | R₁ | R₂ | R₄ |
| 348 | CH₂ | 1 | O | H | 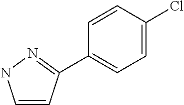 | H |
| 349 | CH₂ | 1 | O | H | 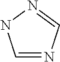 | H |
| 350 | CH₂ | 1 | O | H | 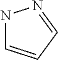 | 12-Br |
| 351 | CH₂ | 1 | O | 8-OCH₃ | 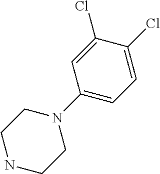 | H |
| 352 | CH₂ | 1 | O | 8-OCH₃ | 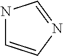 | 12-Br |
| 353 | CH₂ | 1 | O | 8-OCH₃ | 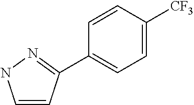 | 12-Br |
| 354 | CH₂ | 1 | O | 8-Br | 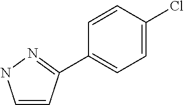 | 12-Br |
| 355 | CH₂ | 1 | O | 8-Br | 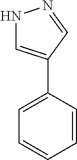 | 12-Br |
| 356 | CH₂ | 1 | O | 8-Br | 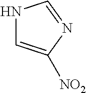 | 12-OH |
| 357 | CH₂ | 1 | O | 8-Br | 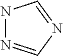 | 12-NO₂ |
| 358 | CH₂ | 1 | O | 8-Br | 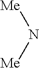 | 12-NH₂ |
| 359 | CH₂ | 1 | O | 8-Br | 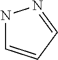 | 12-Br |
| 360 | CH₂ | 1 | O | 8-OH | 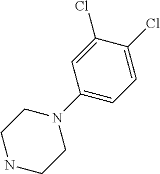 | 12-Br |

TABLE 9
Description of the substituent variation in compounds
prepared with the general formula IX
IX

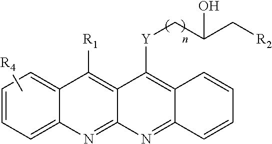

| Serial | | | | | |
|---|---|---|---|---|---|
| No | n | Y | R₁ | R₂ | R₄ |
| 361 | 1 | O | Cl | 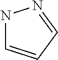 | Br |
| 362 | 1 | O | Cl | 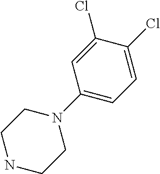 | Br |
| 363 | 1 | O | Cl | 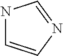 | Br |
| 364 | 1 | O | Cl | 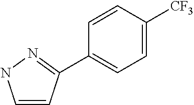 | Br |
| 365 | 1 | O | Cl | 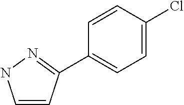 | Br |
| 366 | 1 | O | Cl | 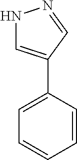 | Br |
| 367 | 1 | O | Cl | 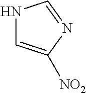 | Br |
| 368 | 1 | O | Cl | 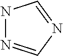 | Br |
| 369 | 1 | O | Cl | 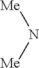 | Br |
| 370 | 1 | O | Cl | 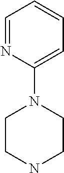 | Br |
| 371 | 1 | O | Cl | 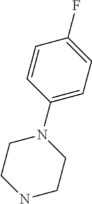 | Br |

TABLE 10
Description of the substituent variation in compounds
prepared with the general formula X
X

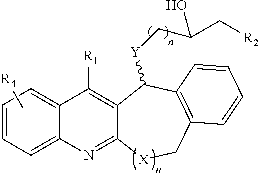

| Serial | | | | | | |
|---|---|---|---|---|---|---|
| No | X | n | Y | R₁ | R₂ | R₄ |
| 372 | N | 1 | O | Cl | 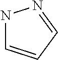 | Br |
| 373 | N | 1 | O | Cl | 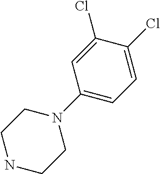 | Br |
| 374 | N | 1 | O | Cl | 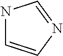 | Br |
| 375 | N | 1 | O | Cl | 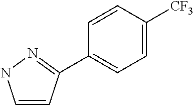 | Br |
| 376 | N | 1 | O | Cl | 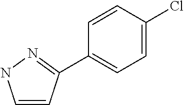 | Br |
| 377 | N | 1 | O | Cl | 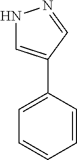 | Br |
| 378 | N | 1 | O | Cl | 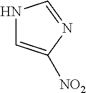 | Br |
| 379 | N | 1 | O | Cl | 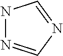 | Br |
| 380 | N | 1 | O | Cl | 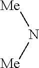 | Br |
| 381 | N | 1 | O | Cl | 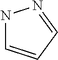 | Br |
| 382 | N | 1 | O | Cl | 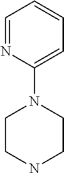 | Br |
| 383 | N | 1 | O | Cl | 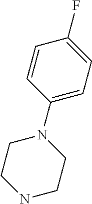 | Br |

Compounds marked with “a” have shown 99% inhibition at <4 μg/ml and described in Table 11.

### Microbiology

These compounds appeared to be endowed with particularly potent and selective anti-mycobacterial activities. Consequently these compounds were tested against drug resistant (MDR and XDR strains included) and intramacrophagic mycobacteria. Most of the strains used were purchased or from clinical origin and were identified by conventional methods (National committee for clinical laboratory standards, 1995, M-24P). The inhibition ability of all compounds was determined for several strains of Mycobacterium such as M. tuberculosis M. fortuitum, M smegmatis, M. marinum, M. gordonae, M. avium, and M. kansasii by the BACTEC460TB method (Heifets, L et al; Antimicrob. Agents Chemother, 40, 1996, 1759-1767, Inderlied, C. B., Salfinger, M., “Antimycrobial agents and susceptibility tests: mycobacteria”, 1996, 1385-1404). Several compounds relates to this invention shown strong inhibitory activity against both M tuberculosis and M. avium, which are two most common mycobacteria causing infection in immunosuppressed patients. Several drug resistant M. tuberculosis strains of clinical origin were collected from various hospitals and their drug resistance was determined by standard methods (Inderlied, C. B., Salfinger, M., “Antimycrobial agents and susceptibility tests: mycobacteria”, 1996, 1385-1404). The inhibition effect of compounds was determined towards sensitive and resistant strains at the single dose of 6.25 mg/ml. Compounds listed in Table 2, 3, 4, 5 A-N, 6, 7, 8, 9 and 10 were screened for antimycobacterial activity and some of the compounds have shown to possess strong inhibitory activity in range of 50-99% against both Mycobacterium tuberculosis and some non tuberculosis mycobacteria.

### Pharmacological Testing

The activity of the compounds of invention to display antimycobacterial activity can be assessed by growth inhibition assays BACTEC 460 TB system, method as shown in the examples given below.

In vitro growth inhibition assay:

The ability of the compounds of present invention to inhibit the growth of Mycobacterium species was determined by the BACTEC 460 TB system. The reference strain M. tuberculosis H37RV ATCC 27294 was grown in Middlebrook 7H9 broth containing 10% supplement at 37° C. on a rotary shaker at 150 rpm for 7 days. The turbidity of the culture was adjusted to 1.0 Mc farland. The middlebrook 7H12B medium vials were seeded with 0.1 ml of the 1.0 Mac farland adjusted M. tuberculosis culture. In the control vials 0.1 ml of the culture was added after 100-fold dilution of the intial inoculam. Stock solution of 1 mg/ml of each compound was prepared in DMSO in separate sterile tubes. The compound was further diluted to concentration of 25 mg/100 ml. 0.1 ml was than added to the 7H 12B vial containing mycobacterial culture so that final concentration of the compound is 6.25 μg/ml. The cap in all the vials were cleaned with isopropyl alcohol and kept in racks. The vials were then incubated at 37° C. without shaking. Test vials were read daily on the BACTAC system till the GI of the control vial reached >30. Once the GI in the control reached 30 GI (GI=GI₍ₙ₎₋GI₍ₙ₋₁₎ was determined for all test and control vials. If GI of test vials is less than that of control vial the culture was sensitive to the test compound. The results were shown in Table 11.

TABLE 11
Antimycobacterial activity of compounds disclosed under this invention

| | | MIC (μg/ml) against | |
|---|---|---|---|
| | | | MDR-TB |
| | Growth inhibition | | ((BTB 08-072) |
| | of M. tuberculosis | M. tuberculosis | This strain |
| Compound | (H37RV | (H37RV | is resistant to |
| No. | ATCC27294) | ATCC27294) | all front line drugs. |
| 5 | + | <6.25 | >6.25 |
| 6 | + | <6.25 | <6.25 |
| 7 | + | <6.25 | >6.25 |
| 8 | + | <6.25 | <6.25 |
| 22 | + | <3.125 | <6.25 |
| 23 | + | <3.125 | >6.25 |
| 26 | + | <3.125 | >4.0 |
| 31 | + | <3.125 | >6.25 |
| 72 | + | <6.25 | <12.5 |
| 76 | + | <6.25 | <6.25 |
| 77 | + | <6.25 | >4.0 |
| 129 | + | <0.39 | <2.0 |
| 134 | + | <6.25 | >6.25 |
| 135 | + | <1.56 | >4.0 |
| 234 | + | <0.78 | >6.25 |
| 235 | + | <6.25 | <6.25 |
| 236 | + | <6.25 | <6.25 |
| 238 | + | <3.125 | <2.0 |
| 241 | + | <3.125 | <2.0 |
| 242 | + | <3.125 | <12.5 |
| Isoniazid | + | 0.25 | >16 |
| Refampin | + | 0.25 | >16 |

There are various compounds disclosed under this invention, listed in the Table 2-10 has shown significant antimycobacterial activity against Mycobacterium tuberculosis under primary screening and these compounds are considered for further evaluation.

### In Vitro Agar Dilution Assay:

MIC of compounds against strains of Mycobacterium were determined by a reference agar dilution method as per the NCCLS-M24-T2 recommendations. The compounds were dissolved in DMSO and diluted twofold to obtain five serial dilutions of each compound. Appropriate volume of compounds were incorporated into duplicate plates of Middlebrook7H10 agar medium supplemented with 10% Middlebrook supplement oleic acid-albumin-dextrose catalase (OADC) enrichment at concentration of 6.25 μg/ml to 0.4 μg/ml. Test organisms (Mycobacterium strains) were grown in Middle brook 7H9 broth containing 0.05% Tween-80 and 10% ADC supplement. After 7 days of incubation at 37° C. the broths were adjusted to the turbidity of 1.0 McFarland standard; the organism were further diluted 10 fold in sterile saline containing 0.10% Tween-80. The resulting mycobacterial suspensions were spotted (2-3 μl/spot) onto drug supplemented 7H10 media plates. The plates were sealed and incubated at 37° C. under 5% CO₂ for 3-4 weeks in upright position. The MIC was recorded as the highest dilution of the drug that completely inhibited the growth of test organisms. Test isolates included a clinical isolate MDR (BTB 08-072) which was found resistant to all front line drugs. Appropriate reference strains and control drug was included in each batch of test.

Apart from that these compounds were screened against various species of Mycobacteria like M. avium-intracellular Complex, M. fortuitum, M. kansasii and different clinical isolates (Table 12). These clinical isolates included 20 isolates that were generally susceptible to common tubercular agents and 10 strains that were resistant to one or more standard antitubercular drugs.

TABLE 12

| | | MIC (μg/mL) | | | | |
|---|---|---|---|---|---|---|
| | | | | M. avium- | | |
| | | M. tuberculosis | | intracellulare | | |
| | Compound | Sensitive | Resistant | Complex | M. fortuitum | M. kansasii |
| Sr. No. | No. | (n = 20) | (n = 10) | (n = 10) | (n = 2) | (n = 2) |
| 1 | 5 | <6.25 | <6.25 | <8.0 | >8.0 | >16.0 |
| 2 | 6 | <6.25 | <6.25 | >8.0 | >8.0 | >16.0 |
| 3 | 7 | <6.25 | <6.25 | >8.0 | >8.0 | >16.0 |
| 4 | 8 | <6.25 | <6.25 | <6.25 | <8.0 | <8.0 |
| 5 | 22 | <3.125 | <4.0 | <2.0 | <4.0 | <4.0 |
| 6 | 23 | <3.125 | <6.25 | <4.0 | <4.0 | <4.0 |
| 7 | 26 | <3.125 | <4.0 | <4.0 | <4.0 | <4.0 |
| 8 | 31 | <6.25 | <6.25 | >8.0 | >8.0 | >8.0 |
| 9 | 72 | <6.25 | <12.5 | >8.0 | >8.0 | >16.0 |
| 10 | 76 | <6.25 | <6.25 | <6.25 | >8.0 | >8.0 |
| 11 | 77 | <6.25 | <4.0 | <6.25 | >8.0 | >8.0 |
| 12 | 129 | <0.39 | <2.0 | <2.0 | <4.0 | <4.0 |
| 13 | 134 | <6.25 | <6.25 | <6.25 | >8.0 | >8.0 |
| 14 | 135 | <1.56 | <4.0 | <2.0 | <2.0 | <2.0 |
| 15 | 234 | <0.78 | <6.25 | <2.0 | <2.0 | <2.0 |
| 16 | 235 | <6.25 | <6.25 | >8.0 | >8.0 | >8.0 |
| 17 | 236 | <6.25 | <6.25 | <8.0 | >8 0 | >16.0 |
| 18 | 238 | <3.125 | <2.0 | <4.0 | <4.0 | <4.0 |
| 19 | 241 | <3.125 | <6.25 | <4.0 | <4.0 | <4.0 |
| 20 | 242 | <3.125 | <12.5 | <4.0 | <4.0 | <4.0 |
| 21 | Isoniazid | 0.25 | >16 | >16 | >16 | >16 |

n—Number of strains tested

1. A compound of general formula I, II, III, IV, V, VI, VII, VIII, IX, X or a tautomer and the stereochemically isomeric 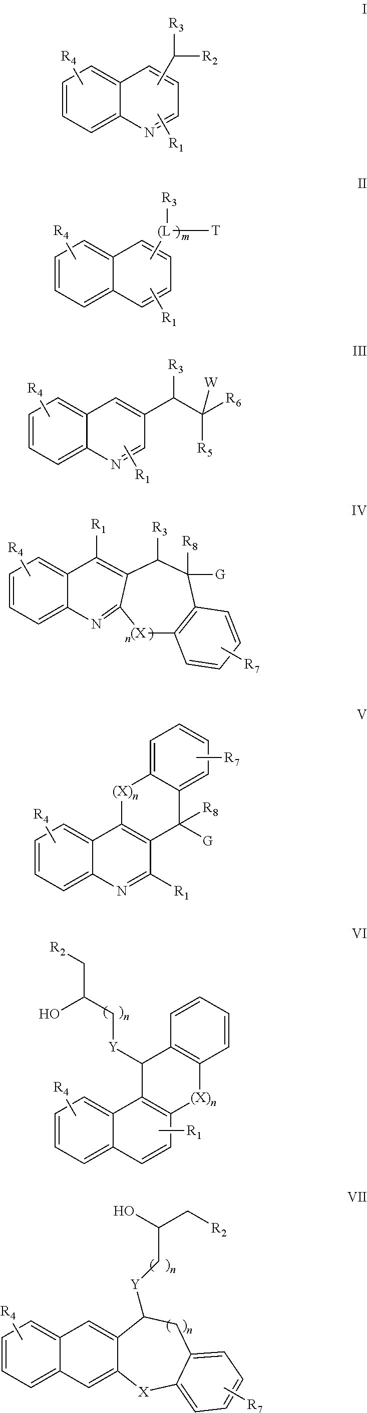 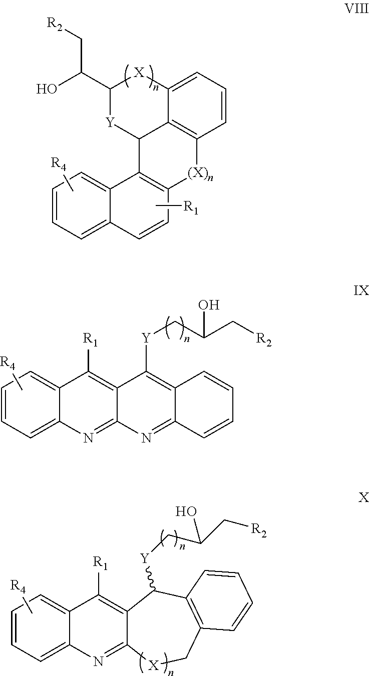
forms thereof or pharmaceutically acceptable salts thereof, a N-oxide form thereof or a pro-drug thereof, wherein all the chemical variations are described in Table 1 below:
TABLE 1
Substitution patterns and Variables, and their Chemical Descriptions
as designated in the general formulae I-X (Figure I)

| Substitution | | |
|---|---|---|
| and Variables | | Chemical Description |
| L | | C, CH or a hetero atom from N, O or S |
| m | | Is an integer 0 to 4 |
| n | | Is an integer 0 to 2 |
| W | | H, OH, COOH, CN, alkoxy |
| R₁ | | Hydrogen, halo, halo alkyl, acyl, cyno, hydroxy, aminoalyl, Het, |
| | | Heterocyclic amines i.e pyrolidinyl, pyrrolyl, pyrrolinyl, |
| | | imidazolidinyl, imidazolyl, pyrazolyl, triazolyl, tetrazolyl, piperidinyl, |
| | | pyridinyl, pyridazinyl, pyrimidinyl, pyrazinyl, trizinyl, morpholinyl |
| | | and thiomorpholinyl, alkyloxy, thio, alkylthio, alkyloxyalkyloxy, |
| | | trifluoroalkyl, trifluoroalkylalkoxy, alkylthioalkyl mono or |
| | | dialkylamino or a radical formula |
| | | 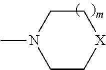 |
| | X | C═O, CH₂, O, S, SO, SO₂, NH, N-alkyl or N-aryl of formula |
| | | 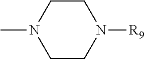 |
| | R₉ | Wherein, R₉ is phenyl which is unsubstituted or substituted with 1-2 |
| | | substituents each independently selected from the group consisting of |
| | | halogen, C₁-C₄ alkyl, C₁-C₄ alkoxy, acyl, cyano, C₁-C₄ thioalkoxy, |
| | | nitro, amino, haloalkyl, haloalkoxy etc.; unsubstituted or substituted |
| | | benzyl; unsubstituted or substituted heteroaryl; unsubstituted or |
| | | substituted heteroaroyl or unsubstituted or substituted diphenyl |
| | | methyl, unsubstituted or substituted naphthyl |
| R₂ | | Is selected from the group of pyrolidinyl pyrrolyl, pyrrolinyl, |
| | | imidazolidinyl, imidazolyl, pyrazolyl, triazolyl, tetrazolyl, piperidinyl, |
| | | pyridinyl, pyridazinyl, pyrimidinyl, pyrazinyl, trizinyl, morpholinyl |
| | | and thiomorpholinyl, optionally substituted with alkyl, haloalkyl, |
| | | hydroxy, alkoxy, amino, mono- or dialkylamino, acyl, nitro, cyano, |
| | | alkylthio, alkyloxyalkyl, alkylthioalkyl, pyrimidinyl and substituted |
| | | piperazine, unsubstituted or substituted pyrazoles that can be |
| | | represented with Figure 2. |
| | | 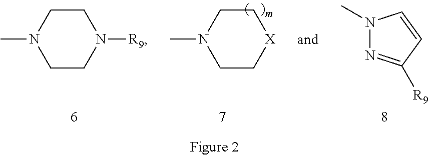 |
| | | R₉, m and X as explained for R₁ |
| T | | Is described by, |
| | | 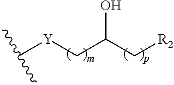 |
| | | Wherein: |
| | p | Is an integer from 0-4 |
| | Y | Is a heteroatom from the group of N, O, S |
| | | m and R₂ are as explained above in this Table. |
| R₃ | | Is phenyl or substituted phenyl, aryl or unsubstituted or substituted |
| | | heteroaryl, unsubstituted or substituted naphthyl etc. |
| R₄ and R₇ | | Is hydrogen, halo, halo alkyl, cyno, hydroxy, acyl, nitro, Ar, alkyl, |
| | | Het, alkyloxy, thio, alkylthio, alkyloxyalkyloxy, alkylthioalkyl mono |
| | | or dialkylamino or pyrolidinyl pyrrolyl, pyrrolinyl, imidazolidinyl, |
| | | imidazolyl, pyrazolyl, triazolyl, tetrazolyl, piperidinyl, pyridinyl, |
| | | pyridazinyl, pyrimidinyl, pyrazinyl, trizinyl, morpholinyl and |
| | | thiomorpholinyl, optionally substituted with alkyl, haloalkyl, hydroxy, |
| | | alkoxy, amino, mono- or dialkylamino, acyl, nitro, cyano, alkylthio, |
| | | alkyloxyalkyl, alkylthioalkyl, pyrimidinyl and substitute dpiperazine, |
| | | unsubstituted or substituted pyrazoles as per Figure 2. Unsubstituted |
| | | and substituted guanidine derivatives, ureas and thio ureas and |
| | | carbodiimides as per Figure 3. |
| | | 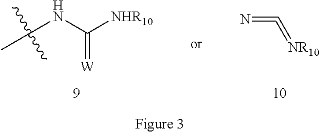 |
| | | Wherein, |
| | | W is O, S, NH |
| | | R₁₀ is H, Substituted or unsubstituted aryl, alkyl etc. |
| R₅ and R₆ | | When one of R₅ and R₆ is 11, the other is 12 and R₁₁, R₁₂ are selected |
| | | from the groups: |
| | | 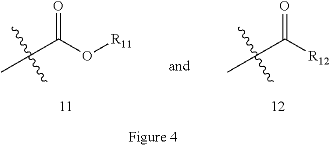 |
| | R₁₁ | Wherein, R₁₁ hydrogen, phenyl that is substituted or unsubstituted |
| | | with 1-2 substituents each independently selected from the group |
| | | consisting of halogen, C₁-C₁₂ alkyl; |
| | | R₁₂ is hydrogen, halo, halo alkyl, cyno, hydroxy, Ar, alkyl, Het, |
| | R₁₂ | alkyloxy, thio, alkylthio, alkyloxyalkyloxy, alkylthioalkyl mono or |
| | | dialkylamino or pyrolidinyl pyrrolyl, pyrrolinyl, imidazolidinyl, |
| | | imidazolyl, pyrazolyl, triazotyl, tetrazolyl, piperidinyl, pyridinyl, |
| | | pyridazinyl, pyrimidinyl, pyrazinyl, trizinyl, morpholinyl and |
| | | thiomorpholinyl, optionally substituted with alkyl, haloalkyl, hydroxy, |
| | | alkoxy, amino, mono- or dialkylamino, acyl, nitro, cyano, alkylthio, |
| | | alkyloxyalkyl, alkylthioalkyl, pyrimidinyl and substituted piperazine, |
| | | unsubstituted or substituted pyrazoles as per Figure 2. |
| R₈ | | When R₈ is hydrogen, halo, halo alkyl, cyno, hydroxy, Ar, alkyl, acyl, |
| | | Het, alkyloxy, thio, alkylthio, alkyloxyalkyloxy, alkylthioalkyl mono |
| | | or dialkylamino or pyrolidinyl pyrrolyl, pyrrolinyl, imidazolidinyl, |
| | | imidazolyl, pyrazolyl, triazolyl, tetrazolyl, piperidinyl, pyridinyl, |
| | | pyridazinyl, pyrimidinyl, pyrazinyl, trizinyl, morplinyl and |
| | | thiomorphlinyl, optionally substituted with alkyl, haloalkyl, hydroxy, |
| | | alkoxy, amino, mono- or dialkylamino, acyl, nitro, cyano alkylthio, |
| | | alkyloxyalkyl, alkylthioalkyl, pyrimidinyl and substituted piperazine, |
| | | unsubstituted or substituted pyrazoles as per Figure 2 then G is from |
| | | subgroup G₁, G₂, G₃, G₄, G₅ and G_{6.} |
| G | | Is a group of different functionality, holds subgroup G₁, G₂, G₃, G₄, |
| | | G₅ and G₆. These subgroups are shown below: |
| | G₁ | When R₈ ≠ H then G = N—O—R₁₃, or G = NH₂, |
| | | R₁₃ is H, alkyl, aryl, substituted aryl, acyl, N, N dimethyl carbamoyl, |
| | | hydrolysable esters, bioesters, phosphonate esters, acyl esters, amino |
| | | acly esters (eg. of hydrophilic and hydrophobic esters), long chain |
| | | hydroxy fatty acids, hydroxy acids (eg. Citric acid), sugar acids (such |
| | | as gluconic acid), sugars like ribose, arabinose, allose, xylose, aldose, |
| | | pyranose, furanose, etc. of formula: |
| | | 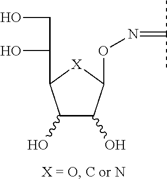 |
| | G₂ | When R₈ = H then G = R₂ and not limited to Pyrolidinyl, pyrrolyl, |
| | | pyrrolinyl, imidazolidinyl, imidazolyl, pyrazolyl, triazolyl, tetrazolyl, |
| | | piperidinyl, pyridinyl, pyridazinyl, pyrimidinyl, pyrazinyl, trizinyl, |
| | | morpholinyl and thiomorpholinyl, optionally substituted with alkyl, |
| | | haloalkyl, hydroxy, alkoxy, amino, mono- or dialkylamino, acyl, nitro, |
| | | cyano, alkylthio, alkyloxyalkyl, alkylthioalkyl, pyrimidinyl and |
| | | substituted piperazine, unsubstituted or substituted pyrazoles (as per |
| | | Figure 2), substituted or unsubstituted guanidine derivatives, ureas |
| | | and thioureas, substituted and unsubstantiated carbodiimides as per |
| | | Figure 3. |
| | G₃ | When R₈ = H, then G can be represented with formula: |
| | | 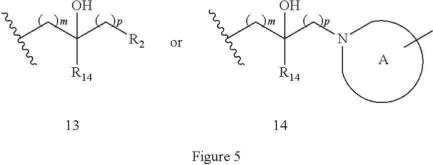 |
| | R₁₄ | R₁₄ Hydrogen, Alkyl substituted or unsubstituted aryl, hetero aryl, |
| | | naphthyl etc. |
| | | m and p are integers 0 to 4 |
| | | R₂ is described above in this table. |
| | | Where in ring A (Figure 5) is hetrocyclyl, wherein if said |
| | | hetrocyclyl contains an NH moiety that nitrogen may be optionally |
| | | substituted by a group selected from C₁₋₄ alkyl, C₁₋₄ alkanoyl, C₁₋₄ |
| | | alkylsulphonyl, C₁₋₄ alkoxy carbonyl, carbamoyl, N-(C₁₋₄ alkyl) |
| | | carbamoyl, N,N-(C₁₋₄ alkyl) carbamoyl, benzyl, benzyloxycarbonyl, |
| | | benzoyl and phenyl sulphonyl. |
| | G₄ | When R₈ = CH₃, G = OR₁₃ |
| | | 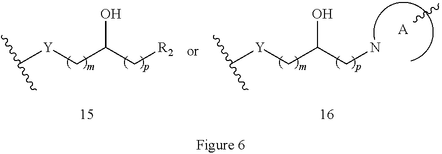 |
| | | R₂, R₁₄, m, p and other chemical variations are same as for G₃ |
| | | Y is same as explained for R₃. |
| | | R₁₃ = Same as defined in G₁ |
| | G₅ | When R₈ = OR₁₅ then G will be |
| | | 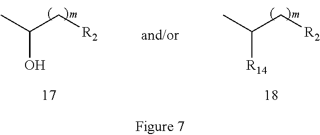 |
| | R₁₅ | Alkyl, substituted or unsubstituted aryl, hetero aryl, naphthyl etc. |
| | | R₂, R₁₄, m, p and other chemical variations are same as in G₃ |
| | G₆ | When R₈ is |
| | | 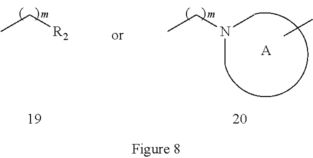 |
| | | Then G is expressed with formula |
| | | 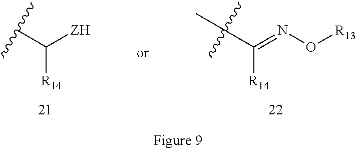 |
| | | R₂, R₁₃, R₁₄, m and other chemical variations are same as in G₃ |
| | | Z is O, S, NH. |
| L | | C, CH or a hetero atom from N, O or S |
| m | | Is an integer 0 to 4 |
| n | | Is an integer 0 to 2 |
| W | | H, OH, COOH, CN, alkoxy |
| R₁ | | Hydrogen, halo, halo alkyl, acyl, cyno, hydroxy, aminoalyl, Het, |
| | | Heterocyclic amines i.e. pyrolidinyl pyrrolyl, pyrrolinyl, |
| | | imidazolidinyl, imidazolyl, pyrazolyl, triazolyl, tetrazolyl, piperidinyl, |
| | | pyridinyl, imidazolidinyl, pyridazinyl, pyrimidinyl, pyrazinyl, trizinyl, |
| | | morpholinyl and thiomorpholinyl, alkyloxy, thio, alkylthio, |
| | X | alkyloxyalkyloxy, trifluoroalkyl, trifluoroalkylalkoxy, alkylthioalkyl |
| | R₉ | mono or dialkylamino or a radical formula |
| | | 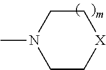 |
| | | CH₂, O, S, SO, SO₂, NH, N-alkyl or N-aryl of formula |
| | | 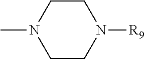 |
| | | Wherein, R₉ is phenyl which is unsubstituted or substituted with 1-2 |
| | | substituents each independently selected from the group consisting of |
| | | halogen, C₁-C₄ alkyl, C₁-C₄ alkoxy, acyl, cyano, C₁-C₄ thioalkoxy, |
| | | nitro, amino, haloalkyl, haloalkoxy etc.; unsubstituted or substituted |
| | | benzyl; unsubstituted or substituted heteroaryl; unsubstituted or |
| | | substituted heteroaroyl or unsubstituted or substituted diphenyl |
| | | methyl, unsubstituted or substituted naphthyl |
| R₂ | | Is selected from the group of pyrolidinyl pyrrolyl, pyrrolinyl, |
| | | imidazolidinyl, imidazolyl, pyrazolyl, triazolyl, tetrazolyl, piperidinyl, |
| | | pyridinyl, imidazolidinyl, pyridazinyl, pyrimidinyl, pyrazinyl, trizinyl, |
| | | morpholinyl and thiomorpholinyl, optionally substituted with alkyl, |
| | | haloalkyl, hydroxy, alkoxy, amino, mono- or dialkylamino, acyl, nitro, |
| | | cyano, alkylthio, alkyloxyalkyl, alkylthioalkyl, pyrimidinyl and |
| | | substituted piperazine, unsubstituted or substituted pyrazoles that can |
| | | be represented with Figure 2. |
| | | 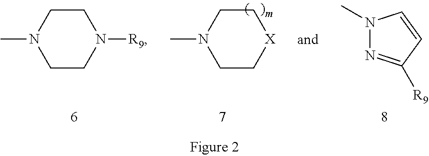 |
| | | R₉, m and X as explained for R₁ |
| T | | Is described by |
| | | 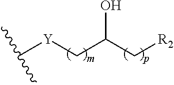 |
| | | Wherein: |
| | p | Is an integer from 0-4 |
| | Y | Is a heteroatom from the group of N, O, S |
| | | m and R₂ are as explained above in this Table. |
| R₃ | | Is phenyl or substituted phenyl, aryl or unsubstituted or substituted |
| | | heteroaryl, unsubstituted or substituted naphthyl etc. |
| R₄ and R₇ | | Is hydrogen, halo, halo alkyl, cyno, hydroxy, acyl, nitro, Ar, alkyl, |
| | | Het, alkyloxy, thio, alkylthio, alkyloxyalkyloxy, alkylthioalkyl mono |
| | | or dialkylamino or pyrolidinyl pyrrolyl, pyrrolinyl, imidazolidinyl, |
| | | imidazolyl, pyrazolyl, triazolyl, tetrazolyl, piperidinyl, pyridinyl, |
| | | imidazolidinyl, pyridazinyl, pyrimidinyl, pyrazinyl, trizinyl, |
| | | morpholinyl and thiomorpholinyl, optionally substituted with alkyl, |
| | | haloalkyl, hydroxy, alkoxy, amino, mono- or dialkylamino, acyl, nitro, |
| | | cyano, alkylthio, alkyloxyalkyl, alkylthioalkyl, pyrimidinyl and |
| | | substitute dpiperazine, unsubstituted or substituted pyrazoles as per |
| | | Figure 2. Unsubstituted and substituted guanidine derivatives, ureas |
| | | and thio ureas and carbodiimides as per Figure 3. |
| | | 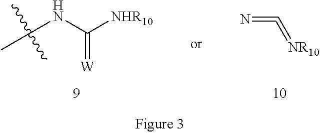 |
| | | Wherein, |
| | | W is O, S, NH |
| | | R₁₀ is H, Substituted or unsubstituted aryl, alkyl etc. |
| R₅ and R₆ | | When one of R₅ and R₆ is 11, the other is 12 and R₁₁, R₁₂ are selected |
| | | from the groups: |
| | | 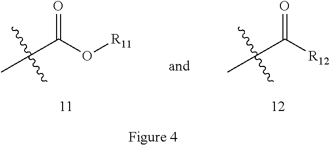 |
| | R₁₁ | Wherein, R₁₁ hydrogen, phenyl that is substituted or unsubstituted |
| | | with 1-2 substituents each independently selected from the group |
| | | consisting of halogen, C₁-C₁₂ alkyl; |
| | R₁₂ | R₁₂ is hydrogen, halo, halo alkyl, cyno, hydroxy, Ar, alkyl, Het, |
| | | alkyloxy, thio, alkylthio, alkyloxyalkyloxy, alkylthioalkyl mono or |
| | | dialkylamino or pyrolidinyl pyrrolyl, pyrrolinyl, imidazolidinyl, |
| | | imidazolyl, pyrazolyl, triazolyl, tetrazolyl, piperidinyl, pyridinyl, |
| | | imidazolidinyl, pyridazinyl, pyrimidinyl, pyrazinyl, trizinyl, |
| | | morpholinyl and thiomorpholinyl, optionally substituted with alkyl, |
| | | haloalkyl, hydroxy, alkoxy, amino, mono- or dialkylamino, acyl, nitro, |
| | | cyano, alkylthio, alkyloxyalkyl, alkylthioalkyl, pyrimidinyl and |
| | | substituted piperazine, unsubstituted or substituted pyrazoles as per |
| | | Figure 2. |
| R₈ | | When R₈ is hydrogen, halo, halo alkyl, cyno, hydroxy, Ar, alkyl, acyl, |
| | | Het, alkyloxy, thio, alkylthio, alkyloxyalkyloxy, alkylthioalkyl mono |
| | | or dialkylamino or pyrolidinyl pyrrolyl, pyrrolinyl, imidazolidinyl, |
| | | imidazolyl, pyrazolyl, triazolyl, tetrazolyl, piperidinyl, pyridinyl, |
| | | imidazolidinyl, pyridazinyl, pyrimidinyl, pyrazinyl, trizinyl, morplinyl |
| | | and thiomorphlinyl, optionally substituted with alkyl, haloalkyl, |
| | | hydroxy, alkoxy, amino, mono- or dialkylamino, acyl, nitro, cyano |
| | | alkylthio, alkyloxyalkyl, alkylthioalkyl, pyrimidinyl and substituted |
| | | piperazine, unsubstituted or substituted pyrazoles as per Figure 2 then |
| | | G is from subgroup G₁, G₂, G₃, G₄, G₅ and G_{6.} |
| G | | Is a group of different functionality, holds subgroup G₁, G₂, G₃, G₄, |
| | | G₅ and G₆. These subgroups are shown below: |
| | G₁ | When R₈ ≠ H then G = N—O—R₁₃ |
| | | R₁₃ is H, alkyl, aryl, substituted aryl, acyl, N, N dimethyl carbamoyl, |
| | | When R₈ ≠ H then G = N—O—R₁₃, G = NH₂, |
| | | R₁₃ H, alkyl, aryl, substituted aryl, acyl, N, N dimethyl carbamoyl, |
| | | hydrolysable esters, bioesters, phosphonate esters, acyl esters, amino |
| | | acly esters (eg. of hydrophilic and hydrophobic esters), long chain |
| | | hydroxy fatty acids, hydroxy acids (eg. Citric acid), sugar acids (such |
| | | as gluconic acid), sugars like ribose, arabinose, allose, xylose, aldose, |
| | | pyranose, furanose, etc. of formula: |
| | | 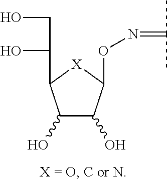 |
| | G₂ | When R₈ = H then G = R₂ and not limited to Pyrolidinyl, pyrrolyl, |
| | | pyrrolinyl, imidazolidinyl, imidazolyl, pyrazolyl, triazolyl, tetrazolyl, |
| | | piperidinyl, pyridinyl, imidazolidinyl, pyridazinyl, pyrimidinyl, |
| | | pyrazinyl, trizinyl, morpholinyl and thiomorpholinyl, optionally |
| | | substituted with alkyl, haloalkyl, hydroxy, alkoxy, amino, mono- or |
| | | dialkylamino, acyl, nitro, cyano, alkylthio, alkyloxyalkyl, |
| | | alkylthioalkyl, pyrimidinyl and substituted piperazine, unsubstituted |
| | | or substituted pyrazoles (as per Figure 2), substituted or unsubstituted |
| | | guanidine derivatives, ureas and thioureas, substituted and |
| | | unsubstantiated carbodiimides as per Figure 3. |
| | G₃ | When R₈ = H, then G can be represented with formula: |
| | | 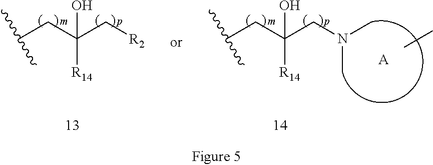 |
| | R₁₄ | R₁₄ Alkyl substituted or unsubstituted aryl, hetero aryl, naphthyl etc. |
| | | m and p are integers 0 to 4 |
| | | R₂ is described above in this table. |
| | | Where in ring A (Figure 5) is hetrocyclyl, wherein if said |
| | | hetrocyclyl contains an NH moiety that nitrogen may be otionally |
| | | substituted by a group selected from C₁₋₄ alkyl, C₁₋₄ alkanoyl, C₁₋₄ |
| | | alkylsulphonyl, C₁₋₄ alkoxy carbonyl, carbamoyl, N-(C₁₋₄ alkyl) |
| | | carbamoyl, N,N-(C₁₋₄ alkyl) carbamoyl, benzyl, benzyloxycarbonyl, |
| | | benzoyl and phenyl sulphonyl. |
| | G₄ | When R₈ = CH₃, G = OR₁₃ |
| | | 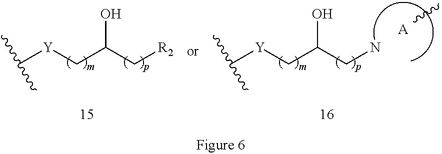 |
| | | R₂, R₁₄, m, p and other chemical variations are same as for G₃ |
| | | Y is same as explained for R₃. |
| | | R₁₃ = Same as defined in G₁ |
| | G₅ | When R₈ = OR₁₅ then G will be |
| | | 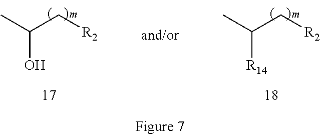 |
| | R₁₅ | Alkyl, substituted or unsubstituted aryl, hetero aryl, naphthyl etc. |
| | | R₂, R₁₄, m, p and other chemical variations are same as in G₃ |
| | G₆ | When R₈ is |
| | | 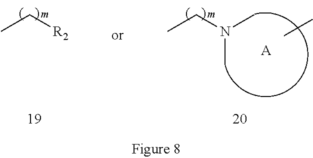 |
| | | Then G is expressed with formula |
| | | 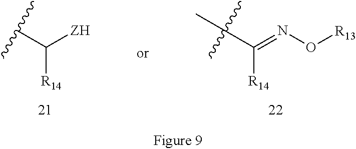 |
| | | R₂, R₁₃, R₁₄, m and other chemical variations are same as in G₃ |
| | | Z is O, S, NH. |

2. The compound of claim 1, generic formula I or a pharmaceutically acceptable salt thereof, which are compounds with general formulae: IA, IB IC and ID 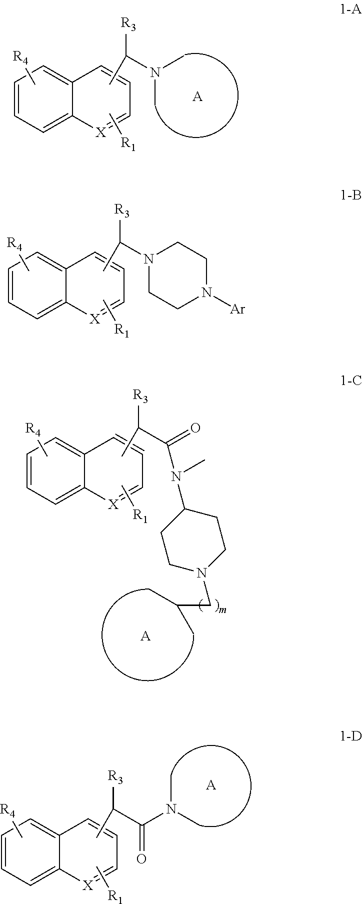
Wherein, all substituents have the same meaning as defined in claim 1, and wherein, the ring A is as defined in table 1.

3. The compound of claim 1, generic formula II or a pharmaceutically acceptable salt thereof, which are compounds with general formulae: IIA; 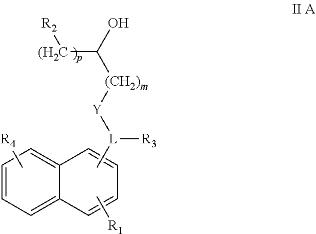
wherein all substituents have the same meaning as defined in claim 1.

4. The compound of claim 1, generic formula III or a pharmaceutically acceptable salt thereof, which is compound of general formulae (IIIA) 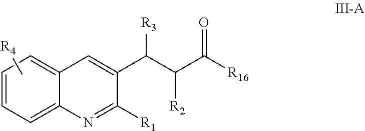
Wherein all substituents have the same meaning as defined in claim 1

5. The compound of claim 1, generic formula IV or a pharmaceutically acceptable salt thereof, which are compounds with general formulae: IV-A, IV-B, IV-C, IV-D, IV-E and IV-F (see Tables 1-10 for all structural variations). 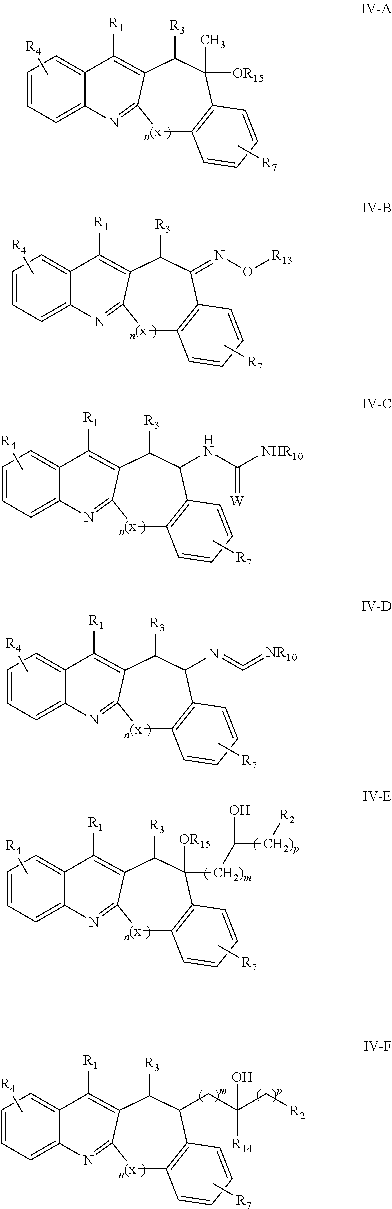
Wherein all substituents have the same meaning as defined in claim 1

6. The compound of claim 1, generic formula V or a pharmaceutically acceptable salt thereof, which are compounds with general formulae: V-A, V-B, V-C, V-D, V-E, V-F, V-G, V-H, V-I, V-J and V-K (see Tables 1-10 for all structural variations). 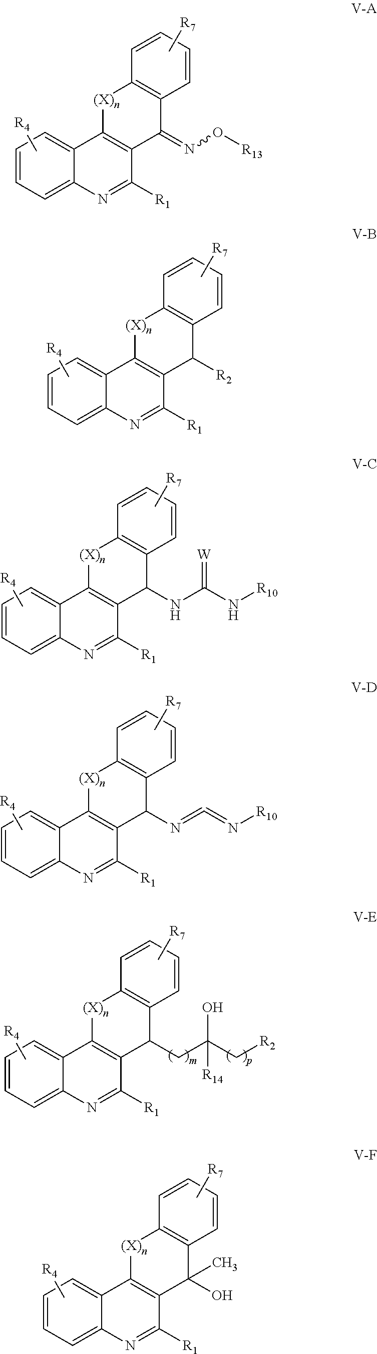 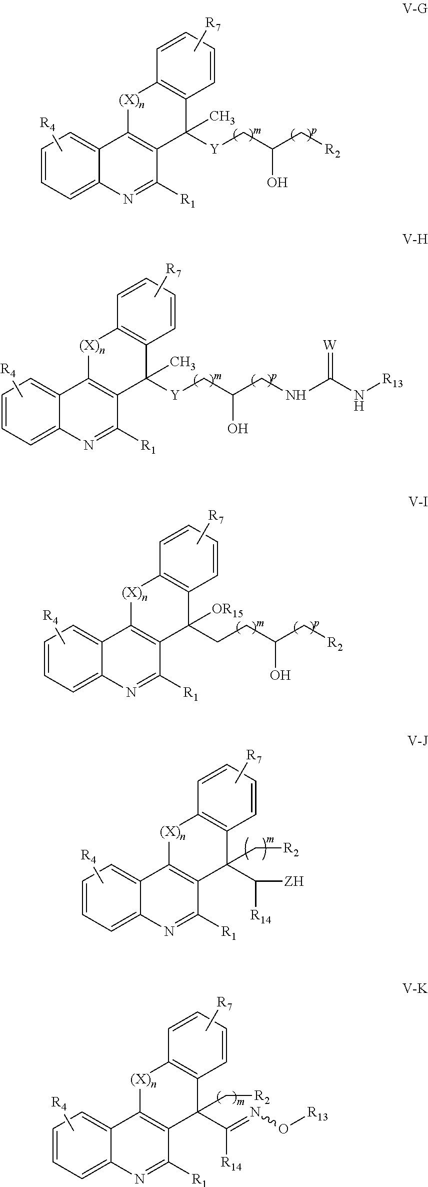
Wherein all substituents have the same meaning as defined in claim 1

7. The compound of claim 1, generic formula VI, or a pharmaceutically acceptable salt thereof, which are compounds with general formulae (VI-A, VI-B and VI-C) (see Tables 1-10 for all structural variations). 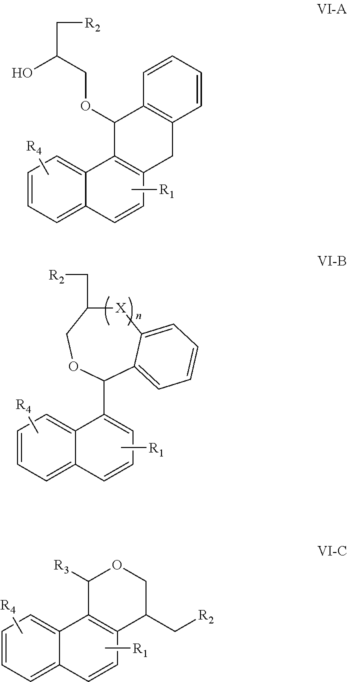
wherein all substituents have the same meaning as defined in claim 1.

8. The compound of claim 1, generic formula VII, or a pharmaceutically acceptable salt thereof, which is compounds with general formulae: (VII-A) (see Tables 1-10 for all structural variations). 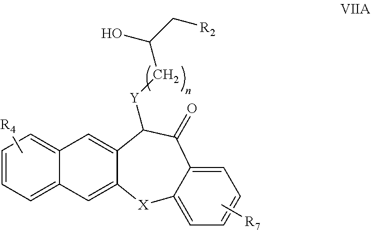
wherein all substituents have the same meaning as defined in claim 1

9. The compound of claim 1, generic formula VIII, or a pharmaceutically acceptable salt thereof, which is compounds with general formulae: (VIII A and VIII B) (see Tables 1-10 for all structural variations). 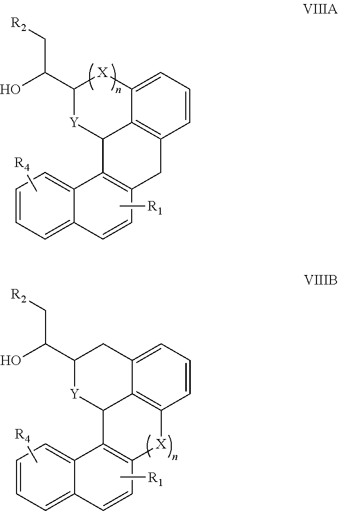
wherein all substituents have the same meaning as defined in claim 1

10. Compound of claim 1, generic formula IX, or a pharmaceutically acceptable salt thereof, which is compounds with general formulae: (IXA, IX B and IX-C) (see Tables 1-10 for all structural variations). 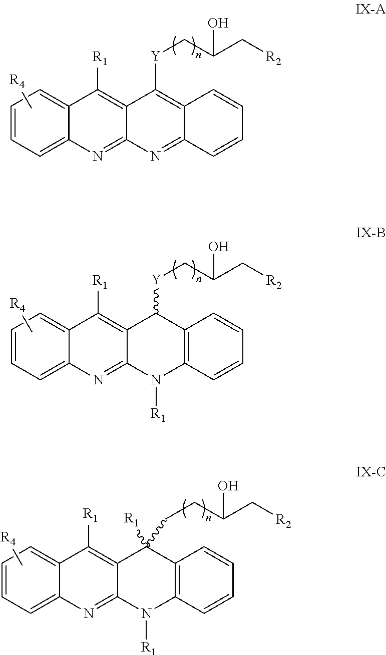
wherein all substituents have the same meaning as defined in claim 1

11. Compound of claim 1, generic formula X, or a pharmaceutically acceptable salt thereof, which is compounds with general formulae: (X-A, X-B, X-C, X-D and X-E) (see Tables 1-10 for all structural variations). 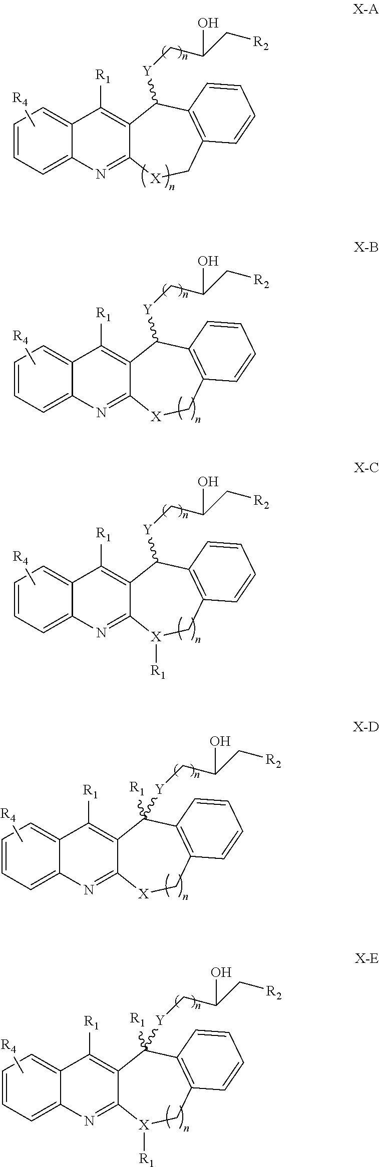
wherein all substituents have the same meaning as defined in claim 1

12. All the compounds, which have been listed in the Tables 2-10 and their possible analogous compounds or a pharmaceutical acceptable salt thereof, a quaternary amine thereof; a stereochemically isomeric forms thereof, a tautomeric form thereof, a N-oxide form thereof or a prodrug thereof.

13. A compound according to anyone claim 1 for use as medicament.

14. A pharmaceutical composition that comprises a compound according to claim 1 or a pharmaceutically acceptable diluent or carrier for the manufacture of medicament for the treatment of mycobacterial disease, which may be caused by any strains of Mycobacterium tuberculosis, including the MDR, and XDR strains etc.

15. A method of treating a mycobacerial infection in warm blooded animal, such as human being, in need of such treatment which comprises administering to the said animal a threpeutically effective amount of a compound according to claim 1.

16. A process of preparing compounds according to claim 1 or pharmaceutically acceptable salts thereof, comprising:
Process (a) for compound of formula II; converting a compound of formula 45 as per Scheme 4 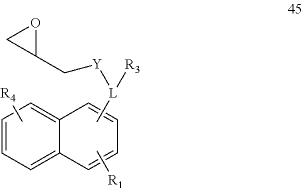
Process (b) for compound of formula III; converting a compound of formula 50 as per Scheme 7 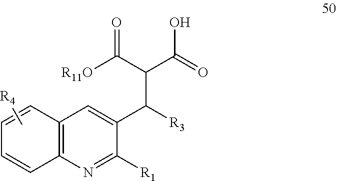
Process (c) for the compound of formula IV; converting a compound of formula 51, 54 and 55 according to Schemes 8, 9 and 10: 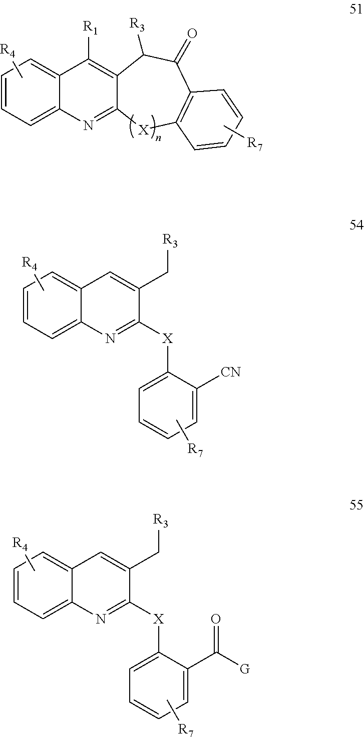
Process (d) for compounds of formula V; converting a compound of formula 57, 64, 70 and 71: 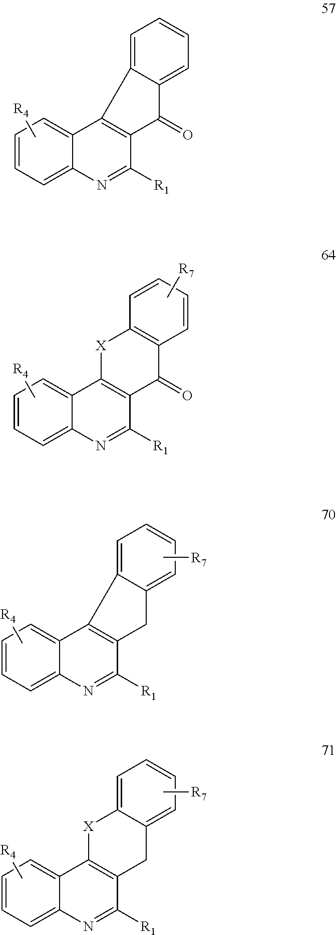
Process (e) for compounds of formula VI; converting a compound of formula 74 and 75: 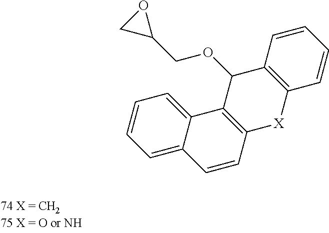
Process (f) for compounds of formula VII; converting a compound of formula 90 and 91: 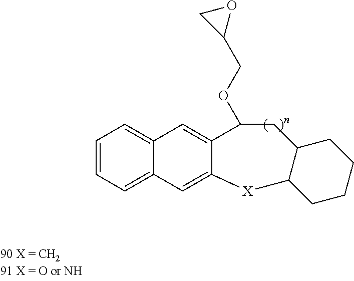
Process (g) for compounds of formula VIII; converting a compound of formula 105, 106 and 107: 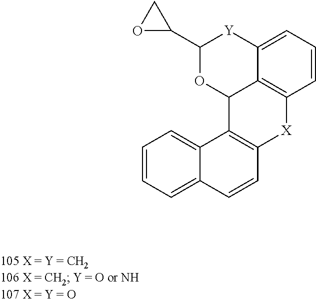
Process (h) for compounds of formula IX; converting a compound of formula 139 as per Scheme 27: 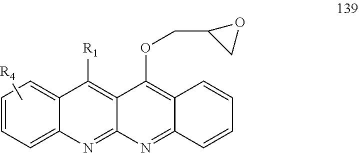
Process (i) for compounds of formula X; conversing a compound of formula 145 as per Scheme 28: 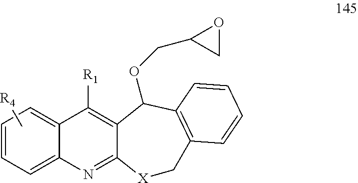
wherein, R₁, R₃, R₄, R₅, R₆, R₇, X, G, T, m, n, p, q and other variables/substitutions are as defined in claim 1.
